(19) United States
(12) Patent Application Publication
(10) Pub. No.: US 2024/0263237 A1
(43) Pub. Date: Aug. 8, 2024

(54) T CELL TRANSCRIPTOMIC PROFILES IN PARKINSON'S DISEASE, AND METHODS AND USES THEREOF

(71) Applicant: La Jolla Institute for Immunology, La Jolla, CA (US)

(72) Inventors: Alessandro Sette, La Jolla, CA (US); Cecilia Lindestam Arlehamn, La Jolla, CA (US)

(73) Assignee: La Jolla Institute for Immunology, La Jolla, CA (US)

(21) Appl. No.: 18/564,599

(22) PCT Filed: May 27, 2022
(86) PCT No.: PCT/US2022/031375

§ 371 Date: Nov. 27, 2023

Related U.S. Application Data
(60) Provisional application No. 63/194,933, filed on May 28, 2021.
(60) Provisional application No. 63/288,323, filed on Dec. 10, 2021.

(51) Int. Cl. C12Q 1/6883

(52) U.S. Cl. CPC C12Q 1/6883; C12Q 2600/158

(57) ABSTRACT

This disclosure provides methods for determining whether a subject is suffering from a neurodegenerative disease, and/or methods of treating a neurodegenerative disease. The disclosed methods comprise detecting differential expression one or more genes or gene products from a sample obtained from the subject.

### CROSS-REFERENCE TO RELATED APPLICATIONS

This application is a U.S. National Phase Application under 35 U.S.C. § 371 of International Application No. PCT/US2022/031375, filed on May 27, 2022, which claims the benefit of and priority to U.S. Patent Application No. 63/194,933, filed on May 28, 2021 and U.S. Patent Application No. 63/288,323, filed on Dec. 10, 2021 the contents of which are incorporated herein by reference in their entirety.

### STATEMENT OF FEDERALLY FUNDED RESEARCH

This invention was made with government support under grant number R01 NS095435 awarded by the National Institutes of Health/NIAID. The government has certain rights in the invention.

### FIELD

The present invention relates in general to the field of neurodegenerative disorder, and more particularly, to the use of T cell subsets and a specific Parkinson's Disease (PD) associated signature informing the diagnosis and/or presence of PD. It moreover pertains to methods of using these signatures, the genes or proteins expressed therefrom, the surface and/or secreted proteins of these cells, or the cell population(s) themselves as therapeutic targets or compositions to prevent or treat neurodegenerative disorder, specifically PD.

### BACKGROUND

Parkinson's disease (PD) is a progressive neurodegenerative disorder characterized by two hallmarks: (i) loss of dopaminergic neurons in the substantia nigra (SN) of the brain responsible for the motor features (Fahn and Sulzer, 2004) and (ii) excess accumulation of aggregated α-synuclein (α-syn) protein (Spillantini et al., 1997). This loss of dopaminergic neurons in the SN is believed to be the reason for the parkinsonian motor signs (increased rigidity, slowness, rest tremor, and at later stages postural instability) observed in PD (Archibald et al., 2013). There are approximately 1 million people in North America affected with this debilitating disease (Marras et al., 2018). The diagnosis and management of PD is challenging as the disease is constrained by limited treatment options, which are mainly focused on improving postural instability and non-motor (constipation, mood, sleep, cognition) symptoms. Considering the increasing prevalence and overall societal impact of PD, it is imperative to explore the underlying mechanisms that play a role in the progression of this heterogenous and complex disease and ultimately to develop targeted symptomatic and disease-modifying interventions.

There is a need in the art to determine a detectable cell signature for the efficient diagnosis of patients that are either to develop or have PD, as well as an unmet need in the art for therapeutic methods and treatments directed to preventing, reducing, or reversing the symptoms and conditions associated with neurodegenerative disorder.

### SUMMARY

Parkinson's disease (PD) is a multi-stage neurodegenerative disorder with largely unknown etiology. Recent findings have identified PD-associated autoimmune features including roles for T cells. To further characterize the role of T cells in PD, the inventors performed RNA sequencing on PBMC and peripheral CD4 and CD8 memory T cell subsets derived from PD patients and age-matched healthy controls. When the groups were stratified by their T cell responsiveness to alpha-synuclein (α-syn) as a proxy for ongoing inflammatory autoimmune response, the study revealed a broad differential gene expression profile in memory T cell subsets and a specific PD associated gene signature.

Applicant identified a significant enrichment of transcriptomic signatures previously associated with PD, including for oxidative stress, phosphorylation, autophagy of mitochondria, cholesterol metabolism and inflammation, and the chemokine signaling proteins CX3CR1, CCR5 and CCR1. In addition, the inventors identified genes in these peripheral cells that have previously been shown to be involved in PD pathogenesis and expressed in neurons, such as LRRK2, LAMP3, and aquaporin. Together, these findings suggest that features of circulating T cells with α-syn-specific responses in PD patients provide insights into the interactive processes that occur during PD pathogenesis and suggest potential intervention targets.

The invention is based, in part, on the role of certain genes in the development, diagnosis, or treatment of neurodegenerative disorder. As broadly described herein, a method of detecting a neurodegenerative disorder is provided, comprising: obtaining a biological sample from a subject; and detecting whether the cell signature or certain genes provided herein are present or differentially expressed in the biological sample by contacting the biological sample with one or more agents capable of detecting the activity, expression, or products of said genes, and determining from said comparison whether a person has or is likely to develop the neurodegenerative disorder.

This disclosure provides methods for diagnosing and treating neurodegenerative disorders or diseases, e.g., Parkinson's Disease (PD). As disclosed in more detain herein, this disclosure provides a method for treating a neurodegenerative disorder in a subject having differential expression of at least one gene or gene product as set forth in Table 1 or Table 2 comprising, or alternatively consisting essentially of, or consisting of identifying a subject having differential expression of the at least one gene or gene product by detecting differential expression of at the least one gene or gene product in a sample obtained from the subject. The method further comprises, or consists of, or consists of administering a treatment or therapy for a neurodegenerative disorder to the subject identified as having differential expression of the at least one gene or gene product. In one aspect, differential expression comprises the expression of the at least one of the genes or gene products as compared to the expression level of the gene or gene product in a healthy subject or control. In one aspect, the neurodegenerative disorder is Alzheimer's Disease (AD), Parkinson's Disease (PD), Tauopathy, Lewy Body Dementia, or Amyotrophic Lateral Sclerosis (ALS) or motor neuron disease.

In one aspect the gene or gene product comprises, consists of, or consists essentially of LSMEM1, AIG1, APOL1, ABCD2, CELSR2, LEAP2, GDF11, LYPD8, CALCRL, NTSR1, AC007040.2, OR1L8, CCR1, CFP, TNFSF13B, ADM5, LYZ, LGALS3BP, LMO7, RNF152, KCNH4, ABCC3, FFAR3, CD300LB, COL16A1, CPB2, IL22, IGFBP6, ACAN, KCNQ4, PAQR4, VAMP4, CNIH2, CX3CR1, CCR5, CCR1, TFEB, SNCA, PARK2, PRKN, UBAP1L, septin 5, GDNF receptor, monoamine oxidase S, aquaporin, LAMP3, polo-like kinase 1, myeloperoxidase, or LRRK2. In yet another aspect, aspect the gene or gene product comprises, consists of, or consists essentially of LSMEM1, AIG1, APOL1, ABCD2, CELSR2, LEAP2, GDF11, LYPD8, CALCRL, NTSR1, AC007040.2, OR1L8, CCR1, CFP, TNFSF13B, ADM5, LYZ, LGALS3BP, LMO7, RNF152, KCNH4, ABCC3, FFAR3, CD300LB, COL16A1, CPB2, IL22, IGFBP6, ACAN, KCNQ4, PAQR4, VAMP4, or CNIH2. In yet another aspect, the gene or gene product comprises, consists of, or consists essentially of CX3CR1, CCR5 or CCR1. In yet another aspect, the gene or gene product comprises, consists of, or consists essentially of TFEB, SNCA, PARK2, PRKN, UBAP1L, septin 5, GDNF receptor, monoamine oxidase S, aquaporin, LAMP3, polo-like kinase 1, myeloperoxidase, or LRRK2. In yet another aspect, the gene or gene product comprises, consists of, or consists essentially of PRKN or LRRK2. In yet another aspect, the gene or product comprises, consists of, or consists essentially of TFEB or UBAPIL.

In one aspect, the subject is a mammal. In yet another aspect, the mammal is selected from an equine, bovine, canine, feline, murine, or a human. In yet another aspect, the subject is a human.

In one aspect, the treatment or therapy comprises surgery, or comprises administration of an immunotherapy, or the administration an agonist or an antagonist of an immune response. In another aspect, the immunotherapy comprises, consists of, or consists essentially of adoptive cell therapy. In one aspect, the adoptive cell therapy comprises, consists of, consists essentially of adoptive cell therapy comprises administering a population of engineered cells. In yet another aspect the antagonist or agonist comprises, consists of, or consists essentially of antagonist or agonist comprises an antibody, a small molecule, a protein, a peptide, an antisense nucleic acid or an aptamer, including an antibody-small molecule conjugate, a bispecific antibody or bispecific molecule. In yet another aspect, the treatment or therapy comprises, consists of, or consists essentially of administration of an anti-TNF therapy. In yet another aspect, the treatment or therapy comprises, consists of, or consists essentially of administration of a dopamine promoter, an antidepressant, a cognition-enhancing medication, an anti-tremor medication, an anticholinergic, a Mao-B inhibitor, or a COMT inhibitor.

In one aspect, the sample is a blood sample. In yet another aspect, the sample comprises, consists of, or consists essentially of a peripheral blood mononuclear cell (PBMCs), a CD4 memory T cell, or a CD8 memory T cell.

In one aspect, the gene or gene product comprises, consists of, or consists essentially of a protein or an mRNA.

In one aspect, the step of identifying comprises, consists of, or consists essentially of determining the level of expression of one or more RNA or gene or gene products listed in Table 3 or Table 4 or the protein product thereof. In yet another aspect, the expression of the one or more RNA or gene or protein product thereof is at least 2.5 fold, at least 3 fold, at least 3.5 fold, at least 4.5 fold, at least 5 fold, at least 6 fold, at least 7 fold, at least 8 fold, at least 9 fold, at least 10 fold, at least 11 fold, at least 12 fold, at least 13 fold, at least 14 fold, or at least 15 fold, compared to a control sample. In yet another aspect, the method further comprises determining the expression level of one or more of two or more, three or more, or four or more, or five or more, or six or more, or seven or more, or eight or more, or nine or more, or ten or more, or eleven or more, or twelve or more, or thirteen or more, or fourteen or more, or fifteen or more, or sixteen or more, or seventeen or more, or eighteen or more, or nineteen or more, or twenty or more, or twenty-one or more, or twenty-two or more, or twenty-three or more, or all of the RNAs or genes or gene products thereof.

In one aspect, the differential expression of the gene is determined by a method comprising measuring mRNA encoding the protein, in situ hybridization, northern blot, PCR, quantitative PCR, RNA-seq, a microarray, differential gene expression analysis (DEseq), gene set enrichment analysis (GSEA), comprises surfaceome analysis or secretome analysis.

In one aspect, this disclosure provides a method for treating a neurodegenerative disorder in a subject having differential expression of at least one of LMO7, LSMEM1, AIG1, APOL1, ABCD2, CELSR2, LEAP2, GDF11, or LYPD8 comprising: identifying a subject having differential expression of the at least one gene or gene product by detecting differential expression of at least one of LMO7, LSMEM1, AIG1, APOL1, ABCD2, CELSR2, LEAP2, GDF11, or LYPD8 in a sample obtained from the subject and administering a treatment or therapy for a neurodegenerative disorder to the subject identified as having differential expression of the at least one gene or gene product. In yet another aspect, the differential expression comprises the upregulation of LMO7, LSMEM1, AIG1, APOL1, ABCD2, CELSR2, LEAP2, GDF11, or LYPD8 in a sample of CD4 T cells obtained from the subject compared to expression in a control sample.

In one aspect, this disclosure provides a method for treating a neurodegenerative disorder in a subject having differential expression of at least one of LMO7, CALCRL, NTSR1, AC007040.2, OR1L8, CCR1, CFP, TNFSF13B, ADM5, LYZ, or LGALS3BP comprising identifying a subject having differential expression of the at least one gene or gene product by detecting differential expression of at least one of LMO7, CALCRL, NTSR1, AC007040.2, OR1L8, CCR1, CFP, TNFSF13B, ADM5, LYZ, or LGALS3BP in a sample obtained from the subject and administering a treatment or therapy for a neurodegenerative disorder to the subject identified as having differential expression of the at least one gene or gene product. In yet another aspect, the differential expression comprises the upregulation of LMO7, CALCRL, NTSR1, AC007040.2, OR1L8, CCR1, CFP, TNFSF13B, ADM5, LYZ, or LGALS3BP in a sample of CD8 T cells obtained from the subject compared to expression in a control sample.

In one aspect, this disclosure provides a method for treating a neurodegenerative disorder in a subject having differential expression of at least one of RNF152, KCNH4, ABCC3, FFAR3, CD300LB, COL16A1, CPB2, 11L22, IGFBP6, or ACAN comprising identifying a subject having differential expression of the at least one gene or gene product by detecting differential expression of at least one of RNF152, KCNH4, ABCC3, FFAR3, CD300LB, COL16A1, CPB2, IL22, IGFBP6, or ACAN in a sample obtained from the subject administering a treatment or therapy for a neurodegenerative disorder to the subject identified as having differential expression of the at least one gene or gene product. In yet another aspect, the differential expression comprises the downregulation of RNF152, KCNH4, ABCC3, FFAR3, CD300LB, COL16A1, CPB2, IL22, IGFBP6, or ACAN in a sample of CD4 T cells obtained from the subject compared to a control sample.

In one aspect, this disclosure provides a method for treating a neurodegenerative disorder in a subject having differential expression of at least one of KCNQ4, PAQR4, VAMP4 or CNIH2 comprising identifying a subject having differential expression of the at least one gene or gene product by detecting differential expression of at least one of KCNQ4, PAQR4, VAMP4 or CNIH2 in a sample obtained from the subject and administering a treatment or therapy for a neurodegenerative disorder to the subject identified as having differential expression of the at least one gene or gene product. In yet another aspect, the differential expression comprises the downregulation of KCNQ4, PAQR4, VAMP4 or CNIH2 in a sample of CD8 T cells obtained from the subject compared to a control sample.

In one aspect, this disclosure provides a method for treating a neurodegenerative disorder in a subject identified as having differential expression of at least one of the genes or gene products selected from the group of LSMEM1, AIG1, APOL1, ABCD2, CELSR2, LEAP2, GDF11, LYPD8, CALCRL, NTSR1, AC007040.2, OR1L8, CCR1, CFP, TNFSF13B, ADM5, LYZ, LGALS3BP, LMO7, RNF152, KCNH4, ABCC3, FFAR3, CD300LB, COL16A1, CPB2, IL22, IGFBP6, ACAN, KCNQ4, PAQR4, VAMP4, or CNIH2 comprising administering a treatment or therapy for the neurodegenerative disorder to the subject.

In one aspect, this disclosure provides a method for treating a neurodegenerative disorder in a subject having differential expression of at least one of the genes or gene products selected from the group of CX3CR1, CCR5 or CCR1, comprising administering a treatment or therapy for a neurodegenerative disorder to the subject.

In one aspect, this disclosure provides a method for treating a neurodegenerative disorder in a subject having differential expression of at least one of the genes or gene products selected from the group of TFEB, SNCA, PARK2, PRKN, UBAPIL, septin 5, GDNF receptor, monoamine oxidase S, aquaporin, LAMP3, polo-like kinase 1, myeloperoxidase, or LRRK2, comprising administering a treatment or therapy for a neurodegenerative disorder to the subject.

In one aspect, this disclosure provides a method for treating a neurodegenerative disorder in a subject having differential expression of at least one of the genes or gene products selected from the group of PRKN, LRRK2, TFEB or UBAPIL, comprising administering a treatment or therapy for a neurodegenerative disorder to the subject

In one aspect, this disclosure provides a method for treating a neurodegenerative disorder in a subject having differential expression of at least one of the genes or gene products selected from the group of PRKN or LRRK2, comprising administering a treatment or therapy for a neurodegenerative disorder to the subject.

In one aspect, this disclosure provides a method for treating a neurodegenerative disorder in a subject having differential expression of at least one of the genes or gene products selected from the group of TFEB or UBAPIL, comprising administering a treatment or therapy for a neurodegenerative disorder to the subject.

In one aspect, this disclosure provides a method for treating a neurodegenerative disorder in a subject having differential expression of CCR5, comprising administering a treatment or therapy for a neurodegenerative disorder to the subject. In yet another aspect, the method comprises a step of detecting CCR5 in a sample of PBMCs obtained from the subject.

In one aspect, this disclosure provides a method for treating a neurodegenerative disorder in a subject having differential expression of CX3CR1, comprising administering a treatment or therapy for a neurodegenerative disorder to the subject. In yet another aspect, the method comprises a step of detecting CX3CR1 in a sample of memory CD4 T cells obtained from the subject.

In one aspect, this disclosure provides, a method for treating a neurodegenerative disorder in a subject having differential expression of CCR1, comprising administering a treatment or therapy for a neurodegenerative disorder to the subject. In yet another aspect, the method comprises a step of detecting CCR1 in a sample of memory CD8 T cells obtained from the subject.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with any accompanying Figures.

### BRIEF DESCRIPTION OF THE FIGURES

FIGS. 1A and 1B show classification of PD and age-matched HC based on the α-syn T cell response. (1A) Violin plot shows the magnitude of T cell response (sum of IFN-γ, IL-5 and IL-10) in HC non-responders (HC_NR) (n=20) PD responders (PD_R) (n=15) and PD non-responders (PD_NR) (n=21). Dotted line denotes the cut off value of 250 SFC. Two-tailed Mann-Whitney, **** p<0.0001 (1B) The gating strategy adopted to identify and sort PBMC, CD4 and CD8 memory T cells from PD and HC subjects.

FIGS. 2A-2C show α-syn specific T cell reactivity is associated with a unique gene expression profile. Volcano plots show log₂ fold change versus −log₁₀ (P value) for the PD_R (n=15) versus PD_NR (n=21) and PD_R versus HC_NR (n=20) respectively. The subset of genes with an absolute log 2 fold change >1.5 and adjusted p-value less than 0.05 were considered significant and are indicated by dotted lines. Black dots of volcano plots indicate protein coding genes upregulated in PD_R and gray dots indicate protein coding genes down-regulated in PD_NR or HC_NR. PCA plots show distinct clusters of PD_R, PD_NR and HC_NR (2A) PBMC (2B) CD4 memory T cells (2C) CD8 memory T cells based on differentially expressed protein coding genes.

FIGS. 3A and 3B show GSEA of the protein coding transcriptome of PD_R vs PD_NR and PD_R vs. HC_NR reveals enrichment of PD associated gene signature in CD4 and CD8 memory T cells. (3A) GSEA for the KEGG PD gene set. The y-axis of the plot shows the enrichment score (ES) for the gene set as the analysis moves down the ranked list of genes. The direction of the peak shows the degree to which the gene set is represented at the top or bottom of the ranked list of genes. The black bars on the x-axis show where the genes in the ranked list appear. The black portion at the bottom shows genes upregulated in PD_R and gray portions represents the genes downregulated in PD_R (upregulated in HC_NR or PD_NR). q, false discovery rate; NES, normalized enrichment score. (3B) Bubble plot demonstrating the enrichment status of several pathways previously reported to be implicated in PD. The black bubble indicates positive enrichment and gray bubble indicates negative enrichment. The size of the bubble is directly proportional to the normalized enrichment score and the shade of the bubble is proportional to the adjusted p value, where a darker bubble indicates higher significance than the lighter shade.

FIGS. 4A-4C show Relative frequency of different cell subsets in HC_NR, PD_NR and PD_R. (4A). Frequency of major PBMC subsets in HC_NR (left bar and circles), PD_NR (middle bars and circles) and PD_R (right bars and circles) (4B) CD4 memory and (4C) CD8 memory T cells were further evaluated for frequency of naïve, effector memory (Tₑₘ), central memory (T_{cm}) and T_{EMRA} populations. Each point represents a donor. Median±interquartile range is displayed. Anova with multiple comparison Tukey correction.

FIGS. 5A and 5B show Comparison of PD vs HC in PBMCs, CD4 and CD8 memory T cells (A) PCA plot demonstrating distinct profile of PBMCs, CD4 and CD8 memory T cells and no separation between PD and HC_NR in either cell type. (B) Venn diagram demonstrating the overlap between PBMC, CD4 and CD8 memory T cells.

FIGS. 6A and 6B show Gene expression profile of specific DE genes in PBMC, CD4 memory and CD8 memory cell types. (6A) Gene expression values of CCR5, CX3CR1, and CCR1 in counts normalized by sequencing depth calculated by DEseq2 package. (6B) Protein expression as percent frequency of subset measured using flow cytometry. Median interquartile range is shown. Two-tailed Mann-Whitney test.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

The practice of the present disclosure employs, unless otherwise indicated, techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### Definitions

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

The term “identify” or “identifying” is to associate or affiliate a patient closely to a group or population of patients who likely experience the same or a similar clinical response to treatment.

The terms “protein,” “polypeptide” and “peptide” are used interchangeably herein when referring to a gene product.

The term “marker” refers to a clinical or sub-clinical expression of a gene or miRNA of interest.

“Expression” as applied to a gene, refers to the differential production of the miR or mRNA transcribed from the gene or the protein product encoded by the gene. A differentially expressed gene may be over expressed (high expression) or under expressed (low expression) as compared to the expression level of a normal or control cell, a given patient population or with an internal control gene (housekeeping gene). In one aspect, it refers to a differential that is about 1.5 times, or alternatively, about 2.0 times, alternatively, about 2.0 times, alternatively, about 3.0 times, or alternatively, about 5 times, or alternatively, about 10 times, alternatively about 50 times, or yet further alternatively more than about 100 times higher or lower than the expression level detected in a control sample.

In one aspect of the disclosure, a “predetermined threshold level”, “threshold value” is used to categorize expression as high or low. As a non-limiting example of the disclosure, the predetermined threshold level is the measured RNA or gene expression level in a control sample from a subject that does not have or did not develop a neurodegenerative disease

A “predetermined value” for a gene as used herein, is so chosen that a patient with an expression level of that gene higher than the predetermined value is likely to experience a more or less desirable clinical outcome than patients with expression levels of the same gene lower than the predetermined value, or vice-versa. Expression levels of genes, such as those disclosed in the present disclosure, are associated with clinical outcomes. One of skill in the art can determine a predetermined value for a gene by comparing expression levels of a gene in patients with more desirable clinical outcomes to those with less desirable clinical outcomes. In one aspect, a predetermined value is a gene expression value that best separates patients into a group with more desirable clinical outcomes and a group with less desirable clinical outcomes. Such a gene expression value can be mathematically or statistically determined with methods well known in the art.

Alternatively, a gene expression that is higher than the predetermined value is simply referred to as a “high expression”, or a gene expression that is lower than the predetermined value is simply referred to as a “low expression”.

Briefly and for the purpose of illustration only, one of skill in the art can determine a predetermined values by comparing expression values of a gene in patients with more desirable clinical parameters to those with less desirable clinical parameters. In one aspect, a predetermined value is a gene expression value that best separates patients into a group with more desirable clinical parameter and a group with less desirable clinical parameter. Such a gene expression value can be mathematically or statistically determined with methods well known in the art.

In one aspect of the disclosure, RNA or gene expression can be provided as a ratio above the threshold level and therefore can be categorized as high expression or up-regulated, whereas a ratio below the threshold level is categorized as down-regulated or low expression.

In another aspect, “expression” level is determined by measuring the expression level of a gene of interest for a given patient population, determining the median expression level of that gene for the population, and comparing the expression level of the same gene for a single patient to the median expression level for the given patient population. For example, if the expression level of a gene of interest for the single patient is determined to be above the median expression level of the patient population, that patient is determined to have high expression (up-regulated) of the gene of interest. Alternatively, if the expression level of a gene of interest for the single patient is determined to be below the median expression level (down-regulated) of the patient population, that patient is determined to have low expression of the gene of interest.

Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed from its gene and/or translated from its mRNA to produce the polypeptide and/or a fragment thereof. The antisense strand is the complement of such a nucleic acid, and the encoding sequence can be deduced therefrom.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

A “blood cell” refers to any of the cells contained in blood. A blood cell is also referred to as an erythrocyte or leukocyte, or a blood corpuscle. Non-limiting examples of blood cells include white blood cells, red blood cells, and platelets.

“Expression” as applied to a gene, refers to the production of the miR or mRNA transcribed from the gene, or the protein product encoded by the mRNA. The expression level of a gene may be determined by measuring the amount of miR or mRNA or protein in a cell or tissue sample. In one aspect, the expression level of a gene is represented by a relative level as compared to a housekeeping gene as an internal control. In another aspect, the expression level of a gene from one sample may be directly compared to the expression level of that gene from a different sample using an internal control to remove the sampling error.

“Differential expression,” “overexpression” or “underexpression” refers to increased or decreased expression, or alternatively a differential expression, of a gene in a test sample as compared to the expression level of that gene in the control sample. In one aspect, the test sample is a diseased cell, and the control sample is a normal cell. In another aspect, the test sample is an experimentally manipulated or biologically altered cell, and the control sample is the cell prior to the experimental manipulation or biological alteration. In yet another aspect, the test sample is a sample from a patient, and the control sample is a similar sample from a healthy individual or a control. The control can be from a subject not experiencing the disease or condition and therefore “healthy” as compared to the subject being tested or treated. Alternatively, the control can be a value determined from evaluation of several healthy subjects and therefore be a range, an average or a median value that provides a cut off for those who are or are not either at high risk of developing the disease or condition. In a yet further aspect, the test sample is a sample from a patient and the control sample is a similar sample from patient not having the desired clinical outcome. In one aspect the expression level in the control sample is the expression level in a sample from a single individual. In another aspect the expression level in the control sample is the median or average expression level of that gene in samples taken from two or more individuals. In one aspect, the differential expression is about 1.5 times, or alternatively, about 2.0 times, or alternatively, about 2.0 times, or alternatively, about 3.0 times, or alternatively, about 5 times, or alternatively, about 10 times, or alternatively about 50 times, or yet further alternatively more than about 100 times higher or lower than the expression level detected in the control sample. Alternatively, the gene is referred to as “over expressed” or “under expressed”. Alternatively, the gene may also be referred to as “up regulated” or “down regulated”.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine,” “cytidine,” “guanosine,” and “thymidine” are used. It is understood that if the nucleic acid is RNA, a nucleotide having a uracil base is uridine.

The terms “oligonucleotide” or “polynucleotide,” or “portion,” or “segment” thereof refer to a stretch of polynucleotide residues which is long enough to use in PCR or various hybridization procedures to identify or amplify identical or related parts of miR or mRNA or DNA molecules. The polynucleotide compositions of this disclosure include miR, RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

MicroRNAs, miRNAs, or miRs are single-stranded RNA molecules of 19-25 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

When a marker is used as a basis for selecting a patient for a treatment described herein, the marker is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

“An effective amount” intends to indicate the amount of a composition, compound or agent (exosomes) administered or delivered to the subject that is most likely to result in the desired response to treatment. The amount is empirically determined by the patient's clinical parameters including, but not limited to the stage of disease, age, gender and histology.

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

A “composition” is intended to mean a combination of active exosome or population of exosomes and another compound or composition, inert (e.g., a detectable label or saline) or active (e.g., a therapeutic compound or composition) alone or in combination with a carrier which can in one embodiment be a simple carrier like saline or pharmaceutically acceptable or a solid support as defined below.

A “pharmaceutical composition” is intended to include the combination of an active exosome or population of exosomes with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, the disease being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, inhalation, injection, and topical application.

An agent of the present disclosure can be administered for therapy by any suitable route of administration. It will also be appreciated that the preferred route will vary with the condition and age of the recipient, and the disease being treated.

An antibody, as referred to herein, can be a polyclonal or monoclonal antibody, or binding fragment thereof. Antibodies sometimes are IgG, IgM, IgA, IgE, or an isotype thereof (e.g., lgG1, lgG2a, lgG2b or lgG3), sometimes are polyclonal or monoclonal, and sometimes are chimeric, humanized or bispecific versions of an antibody. In some embodiments an antibody or portion thereof, comprises a chimeric antibody, Fab, Fab′, F(ab′)2, Fv fragment, scFv, diabody, aptamer, synbody, camelid, the like and/or a combination thereof.

Methods of the invention include treatment methods, which result in any therapeutic or beneficial effect. As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is neurodegenerative disorder, the following clinical end points are non-limiting examples of treatment: reduction in symptoms, slowing of disease progress, longer overall survival, longer time to end-of life, or prevention of symptoms or conditions related to neurodegenerative disease.

In some embodiments a subject is in need of a treatment, cell or composition described herein. In certain embodiments a subject has or is suspected of having a neurodegenerative disorder. In certain embodiments an engineered T cell described herein is used to treat a subject having, or suspected of having, a neurodegenerative disorder.

The term “treating” as used herein is intended to encompass curing as well as ameliorating at least one symptom of the condition or disease. For example, in the case of liver fibrosis, the term “treatment” intends a more favorable clinical assessment by a treating physician or assistant and/or reduced expression of fibrosis markers, e.g., αSMA, CTGF, collagen, matrix molecules and/or a shift toward normal read-outs in tests that diagnose liver function and/or liver fibrosis. “Treating” as used herein also encompasses prophylactic or preventative treatment including preventing disease or symptoms of a disease, slowing the onset of disease or reducing the severity of a disease or symptoms of a disease.

In some embodiments, presented herein is a method of treating a subject having or suspected of having a neurodegenerative disease. In certain embodiments, a method of treating a subject comprises administering a therapeutically effective amount of an engineered T cell to a subject.

Non-limiting examples of a neurodegenerative disorder include Alzheimer's disease (AD), Parksinson's Disease (PD), Tauopathy, Lewy Body Dementia, or Amyotrophic Lateral Sclerosis (ALS) or motor neuron disease.

In some embodiments, a method inhibits, or reduces relapse or progression of the neurodegenerative disorder.

A therapeutic or beneficial effect of treatment is therefore any objective or subjective measurable or detectable improvement or benefit provided to a particular subject. A therapeutic or beneficial effect can, but need not be, complete ablation of all or any particular adverse symptom, disorder, illness, disease or complication caused by or associated with neurodegenerative disorder pathology. Thus, treatment may be achieved when there is an incremental improvement or a partial reduction in an adverse symptom, disorder, illness, disease or complication caused by or associated with neurodegenerative disorder pathology, or an inhibition, decrease, reduction, suppression, prevention, limit or control of worsening or progression of one or more adverse symptoms, disorders, illnesses, diseases or complications caused by or associated with neurodegenerative disorder pathology, over a short or long duration.

A therapeutic or beneficial effect also includes reducing or eliminating the need, dosage frequency or amount of a second active treatment such as another drug or other agent (e.g., anti-viral) used for treating a subject having or at risk of having a neurodegenerative disorder pathology. For example, reducing an amount of an adjunct therapy, for example, a reduction or decrease of a treatment for neurodegenerative disorder.

In methods in which there is a desired outcome, such as a therapeutic or prophylactic method that provides a benefit from treatment, agonists or antagonists can be administered in a sufficient or effective amount. As used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

Therapy or treatments for neurological diseases, e.g., Parkinson's Disease, include, but are not limited to DOPA decarboxylase inhibitors, DA precursors, COMT inhibitors, inhibitors of the breakdown of Levodopa, DA agonists, MAO-B inhibitors, inhibitors of the breakdown of dopamine, NMDA antagonists, Adenosine 2A antagonists, anticholinergics, deep brain stimulation (DBS), antidepressants, anti-tumors, cognition-enhancing medications, or dopamine promoters.

In some embodiments, an amount sufficient, or an amount effective, is provided in a single administration. In some embodiments, an amount sufficient, or an amount effective, is provided in multiple administrations. In some embodiments, an amount sufficient, or an amount effective, is achieved by agonists or antagonists alone, or in a composition or method that comprises a second active component. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

The term “subject” refers to an animal, typically a mammalian animal (mammal), such as a nonhuman primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans.

Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). Subjects include animal disease models, for example, a mouse model, and other animal models of pathogen infection known in the art. In some embodiments a mammal is a human. A mammal can be any age or at any stage of development (e.g., an adult, teen, child, infant, or a mammal in utero). A mammal can be male or female. A mammal can be a pregnant female. In certain embodiments a mammal can be an animal disease model, for example, animal models used for the study of neurodegenerative disorder.

In some embodiments, subjects appropriate for treatment include those having or at risk of having neurodegenerative disorder pathology.

Treatment of a neurodegenerative disorder can be at any time during the neurodegenerative disorder or corresponding condition. Agonists or antagonists can be administered as a combination (e.g., with a second active), or separately, concurrently or in sequence (sequentially) in accordance with the methods as a single or multiple dose e.g., one or more times hourly, daily, weekly, monthly or annually or between about 1 to 10 weeks, or for as long as appropriate, for example, to achieve a reduction in the onset, progression, severity, frequency, duration of one or more symptoms or complications associated with or caused by neurodegenerative disorder pathology, or an adverse symptom, condition or complication associated with or caused by neurodegenerative disorder. Thus, a method can be practiced one or more times (e.g., 1-10, 1-5 or 1-3 times) an hour, day, week, month, or year. The skilled artisan will know when it is appropriate to delay or discontinue administration. A non-limiting dosage schedule is 1-7 times per week, for 1, 2, 3, 4, 5, 6, 7, 8, 9, 10, 15, 20 or more weeks, and any numerical value or range or value within such ranges.

The exact formulation and route of administration for a composition for use according to the methods of the invention described herein can be chosen by a caregiver (e.g., a medical professional, a physician) in view of the patient's condition. See e.g., Fingl et al. 1975, in “The Pharmacological Basis of Therapeutics,” Ch. 1, p. 1; which is incorporated herein by reference in its entirety. Any suitable route of administration can be used for administration of a compound described herein. Methods of the invention may be practiced by any mode of administration or delivery, or by any route, systemic, regional and local administration or delivery. Exemplary administration and delivery routes include intravenous (i.v.), intraperitoneal (i.p.), intrarterial, intramuscular, parenteral, subcutaneous, intra-pleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, or intralymphatic. Other non-limiting examples of routes of administration include topical or local (e.g., transdermally or cutaneously, (e.g., on the skin or epidermus), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, and the like), by parenteral administration (e.g., parenterally, e.g., intravenously, intra-arterially, intramuscularly, intraperitoneally, intradermally, subcutaneously, intracavity, intracranially, intraarticular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition herein is provided to a subject. A composition that is provided to a subject can be provided to a subject for self-administration or to another (e.g., a caregiver, a medical professional) for administration to a subject. For example, a composition described herein can be provided as an instruction written by a medical practitioner that authorizes a patient to be provided a composition or treatment described herein (e.g., a prescription). In another example, a composition can be provided to a subject wherein the subject self-administers a composition orally, intravenously or by way of an inhaler, for example.

A dose can be administered in an effective amount or an amount sufficient to treat, prevent or slow a virus infection or to treat, prevent or slow one or more adverse symptoms and/or complications. An exact dose can be determined by a caregiver or medical professional by methods known in the art (e.g., by analyzing data and/or the results of a clinical trial).

Doses can be based upon current existing protocols, empirically determined, using animal disease models or optionally in human clinical trials. Initial study doses can be based upon animal studies set forth herein, for a mouse, which weighs about 30 grams, and the amount of agonist or antagonist administered that is determined to be effective. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1 μg/kg-500 mg/kg, 1-10 μg/kg, 10-25 μg/kg, 25-50 μg/kg, 50-100 μg/kg, 100-500 μg/kg, 500-1,000 μg/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually. A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, the onset, progression, severity, frequency, duration probability of or susceptibility of the symptom, condition, pathology or complication, or vaccination or immunization to which treatment is directed, the clinical endpoint desired, previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

Typically, for therapeutic treatment, compositions, agonists or antagonists disclosed herein will be administered as soon as practical, typically within less than 1, 1-2, 2 4, 4-12, 12-24 or 24-72 hours after a subject is suspected of having neurodegenerative disorder, or within less than 1, 1-2, 2-4, 4-12, 12-24 or 24-48 hours after onset or development of one or more adverse symptoms, conditions, pathologies, complications, etc., associated with or caused by neurodegenerative disorder pathology.

The dose amount, number, frequency or duration may be proportionally increased or reduced, as indicated by the status of the subject. For example, whether the subject has a pathogen infection, whether the subject has been exposed to, contacted or infected with pathogen or is merely at risk of pathogen contact, exposure or infection, whether the subject is a candidate for or will be vaccinated or immunized. The dose amount, number, frequency or duration may be proportionally increased or reduced, as indicated by any adverse side effects, complications or other risk factors of the treatment or therapy.

Agonists and antagonists can be incorporated into compositions, including pharmaceutical compositions, e.g., a pharmaceutically acceptable carrier or excipient. Such pharmaceutical compositions are useful for, among other things, administration to a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, ophthalmic, optical, intravenous (i.v.), intramuscular, intraglandular, intraorgan, or intralymphatic.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, ophthalmic, optical, intravenous (i.v.), intramuscular, intraglandular, intraorgan, or intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

Co-solvents may be added to an agonist or antagonist composition or formulation. Non-limiting examples of co-solvents contain hydroxyl groups or other polar groups, for example, alcohols, such as isopropyl alcohol; glycols, such as propylene glycol, polyethylene glycol, polypropylene glycol, glycol ether; glycerol; polyoxyethylene alcohols and polyoxyethylene fatty acid esters. Non-limiting examples of co-solvents contain hydroxyl groups or other polar groups, for example, alcohols, such as isopropyl alcohol; glycols, such as propylene glycol, polyethylene glycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include antibacterial, antiviral, antifungal and anti-parasitics. Antimicrobials include agents and compounds that kill or destroy (-cidal) or inhibit (-static) contamination by or growth, infectivity, replication, proliferation, reproduction of the microbial organism.

Exemplary anti-bacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, ribavirin, valacyclovir, ganciclovir, 1,-D-ribofuranosyl-1,2,4-triazole-3 carboxamide, 9≥2-hydroxy-ethoxy methylguanine, adamantanamine, 5-iodo-2′-deoxyuridine, trifluorothymidine, interferon and adenine arabinoside.

Pharmaceutical formulations and delivery systems appropriate for the compositions and methods of the invention are known in the art (see, e.g., Remington: The Science and Practice of Pharmacy (2003) 20th ed., Mack Publishing Co., Easton, PA; Remington's Pharmaceutical Sciences (1990) 18th ed., Mack Publishing Co., Easton, PA; The Merck Index (1996) 12th ed., Merck Publishing Group, Whitehouse, NJ; Pharmaceutical Principles of Solid Dosage Forms (1993), Technonic Publishing Co., Inc., Lancaster, Pa.; Ansel ad Soklosa, Pharmaceutical Calculations (2001) 11th ed., Lippincott Williams & Wilkins, Baltimore, MD; and Poznansky et al., Drug Delivery Systems (1980), R. L. Juliano, ed., Oxford, N.Y., pp. 253-315).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, numerical values are often presented in a range format throughout this document. The use of a range format is merely for convenience and brevity and should not be construed as an inflexible limitation on the scope of the invention.

Accordingly, the use of a range expressly includes all possible subranges, all individual numerical values within that range, and all numerical values or numerical ranges include integers within such ranges and fractions of the values or the integers within ranges unless the context clearly indicates otherwise. This construction applies regardless of the breadth of the range and in all contexts throughout this patent document. Thus, to illustrate, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth. Further, for example, reference to a series of ranges of 2-72 hours, 2-48 hours, 4-24 hours, 4-18 hours and 6-12 hours, includes ranges of 2-6 hours, 2, 12 hours, 2-18 hours, 2-24 hours, etc., and 4-27 hours, 4-48 hours, 4-6 hours, etc.

As also used herein a series of range formats are used throughout this document. The use of a series of ranges includes combinations of the upper and lower ranges to provide a range. Accordingly, a series of ranges include ranges which combine the values of the boundaries of different ranges within the series. This construction applies regardless of the breadth of the range and in all contexts throughout this patent document. Thus, for example, reference to a series of ranges such as 5-10, 10-20, 20-30, 30-40, 40-50, 50-75, 75-100, 100-150, and 150-171, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-171, and so forth.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions.

This disclosure provides diagnostic methods. As used herein “diagnose” or “diagnosing” includes identifying a subject that will or is likely to develop a neurodegenerative disease or determining if a subject will or is likely to develop a neurodegenerative disease. As used herein “diagnostic” includes products or methods for identifying a subject that will or is likely to develop a neurodegenerative disease or determining if a subject will or is likely to develop a neurodegenerative disease. In one aspect, therapy and a subject's health can be monitored by determining the expression level of one or more RNAs or genes or gene products listed in Tables 3 and 4 in a sample isolated from the subject prior to, during, and/or after the therapy. The method can further comprise, or alternatively consist essentially of, or yet further consist of, determining the expression level of one or more of, two or more, three or more, or four or more, or five or more, or six or more, or seven or more, or eight or more, or nine or more, or ten or more, or eleven or more, or twelve or more, or thirteen or more, or fourteen or more, or fifteen or more, or sixteen or more, or seventeen or more, or eighteen or more, or nineteen or more, or twenty or more, or twenty-one or more, or twenty-two or more, or twenty-three or more, or twenty-four or more, or twenty-five or more, or twenty-six, or twenty-seven or more, or twenty-eight or more, or twenty-nine or more, or thirty or more, thirty-five or more, forty or more, forty-five or more, fifty or more, fifty-five or more of, or all of the RNAs or genes or gene products thereof listed in Tables 3 and 4.

In other aspects, this disclosure provides kits for diagnosing and/or treating neurodegenerative diseases In some embodiments, the kits disclosed herein comprise probes and/or primers to determine the expression profile of one or more of the genes or genes products of LSMEM1, AIG1, APOL1, ABCD2, CELSR2, LEAP2, GDF11, LYPD8, CALCRL, NTSR1, AC007040.2, OR1L8, CCR1, CFP, TNFSF13B, ADM5, LYZ, LGALS3BP, LMO7, RNF152, KCNH4, ABCC3, FFAR3, CD300LB, COL16A1, CPB2, IL22, IGFBP6, ACAN, KCNQ4, PAQR4, VAMP4, CNIH2, CX3CR1, CCR5, CCR1, TFEB, SNCA, PARK2, PRKN, UBAPIL, septin 5, GDNF receptor, monoamine oxidase S, aquaporin, LAMP3, polo-like kinase 1, myeloperoxidase, or LRRK2.

In regards to the kits disclosed herein, in some embodiments, the one or more probes and/or primers are detectably labeled. In a further aspect, the kit further comprises detectable labels that in one aspect are attached to the probes and/or primers, wherein in one aspect, the detectable label is not a polynucleotide. In some embodiments, the probes and/or primers are detectably labeled with an enzymatic, radioactive, fluorescent and/or luminescent moiety. In one aspect, the detectable label is not a polynucleotide that is naturally fluorescent or detectable.

The following examples are intended to illustrate, and not limit, the disclosed herein. For example, while the examples are noted to be for the isolation, purification and use of exosome compositions for the treatment of a fibrotic or liver disease or an associated disorder, the methods and compositions can be modified for the treatment of other fibrotic diseases as noted herein.

### EXAMPLES

### Example 1: Classification of PD Subjects Based on α-Syn Specific T Cell

### Reactivity

In previous studies, the inventors detected α-syn specific T cell responses in approximately 40-50% of PD subjects (Lindestam Arlehamn et al., 2020; Sulzer et al., 2017). The inventors further reported that α-syn specific T cell reactivity is specifically associated with preclinical and early time points (<10 years diagnosis prior to sample donation) following onset of motor PD features (Lindestam Arlehamn et al., 2020), while responses subsided in later stages of PD. Based on this finding, the inventors hypothesized that PD subjects that demonstrate α-syn-specific T cell reactivity could be a “proxy” for individuals associated with an active inflammatory autoimmunity phenotype, and that analysis might reveal a transcriptional profile distinct from subjects without PD (healthy controls; HC) or PD subjects that do not exhibit α-syn T cell reactivity.

Accordingly, based on the magnitude of total response mounted against α-syn peptides, PD subjects were classified in two categories: responders (denoted as PD_R; >250 SFC for the sum of IFNγ, IL-5, and IL-10) and non-responders (denoted as PD_NR; <250 SFC). IFNγ, IL-5, and IL-10 were chosen as markers of T cell reactivity as they capture a broad immune response (i.e. Th1/Th2/Treg) and we have previously shown them to be detected at higher levels in PD [7,8]. The inventors also included age-matched HC who were α-syn non-responders (HC_NR), to avoid the possibility that HC who exhibit α-syn-specific T cell reactivity may be in prodromal stages of PD. The classification criteria were based on previously published studies (Lindestam Arlehamn et al., 2020; Sulzer et al., 2017) where the inventors determined α-syn-specific T cell reactivity for PD following in vitro restimulation assays, and measured cytokine release by Fluorospot or ELISPOT assays.

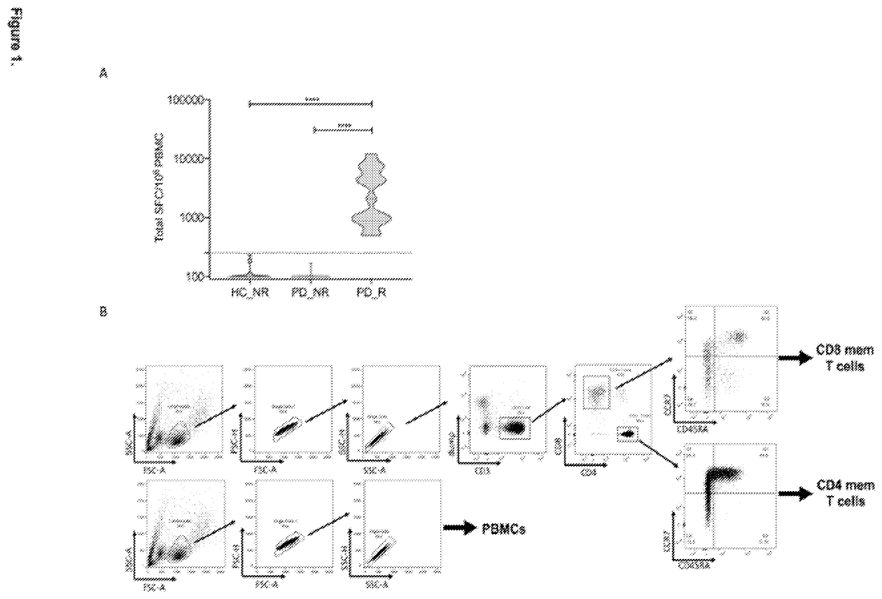

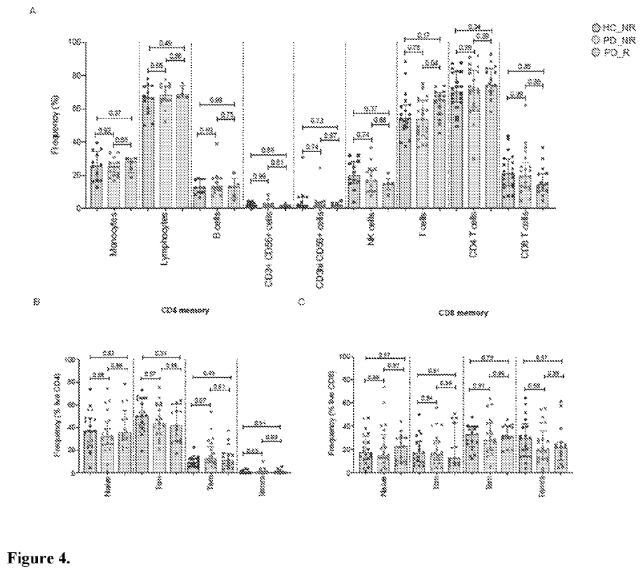

To investigate differential gene expression signatures, the inventors examined 34 PD subjects including PD_R (n=14) and PD_NR (n=20) (FIG. 1A). For control subjects, the inventors selected 19 HC_NR subjects. The inventors first analyzed the relative frequency of major PBMC subsets, i.e., monocytes, NK cells, B cells, T cells, and CD4 and CD8 memory T cells by flow cytometric analysis. The frequency of each PBMC subset was remarkably similar in all groups (FIG. 4A) and there was no significant difference between CD4 and CD8 memory T cell subsets (FIG. 4B-C).

### Example 2: Transcriptional Analysis of PBMC, CD4 and CD8 Memory T Cells in PD and Age-Matched HC

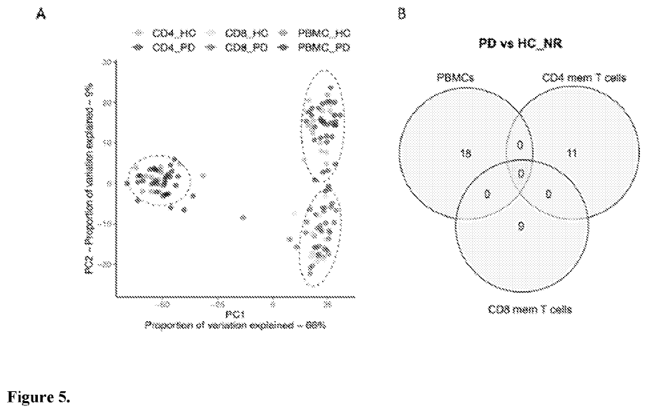

The inventors then examined the hypothesis that the circulating peripheral lymphocytes reflect a general inflammatory state associated with early PD. The inventors analyzed PBMC, CD4 and CD8 memory T cells from PD_R, PD_NR, and HC_NR subjects to for specific transcriptomic signatures that might be associated with PD. The low frequency of α-syn-specific CD4 T cells detected in PBMCs in early PD (Lindestam Arlehamn et al., 2020; Sulzer et al., 2017) requires 2-week in vitro culture to produce sufficient cells for characterization. CD4 and CD8 memory T cell subsets were identified using CCR7 and CD45RA immunolabel and were sorted based on the gating strategy in FIG. 1B. Whole PBMC and sorted CD4 and CD8 memory T cell populations were sequenced with the Smart seq protocol (Picelli et al., 2014). To assess whether differences in gene expression could distinguish the groups, the inventors applied Principal Component Analysis (PCA). As expected, the global gene expression profile analyzed by PCA revealed three distinct clusters corresponding to the PBMC, memory CD4 and memory CD8 T cell subsets However, the same analysis did not discriminate between the PBMC, CD4 or CD8 memory T cells from PD and HC subjects (FIG. 5A).

The inventors next performed differential gene expression analysis (DEseq) comparing PD vs. HC_NR to explore PD-specific gene expression signatures of PBMC, CD4 and CD8 memory T cells. (see RNA-seq analysis methods for data availability). Only 26 genes were differentially expressed in PBMC between PD and HC_NR [fold change ≥1.5 (absolute log 2≥0.58) and adjusted p-value <0.05]. Of the 26 genes, only 18 were protein coding; 7 were up-regulated and 11 down-regulated. (Table 1). A total of 11 genes (1 up-regulated and 10 down-regulated; Table 1) and 9 genes (4 up-regulated and 5 genes down-regulated; Table 1) were differentially expressed protein coding genes in CD4 and CD8 memory T cells, respectively. In conclusion, few genes were differentially expressed at the global level, and the inventors did not identify any specifically molecular pathway that was differentially regulated in PBMC, CD4 or CD8 memory T cells. Moreover, no overlap was observed between the few protein coding genes that were differentially expressed in PD vs. HC_NR, in PBMC, CD4, or CD8 cell subsets (FIG. 5B).

### Example 3: Classification of PD Subjects Based on α-Syn-Specific T Cell Reactivity Reveals Specific Gene Signatures

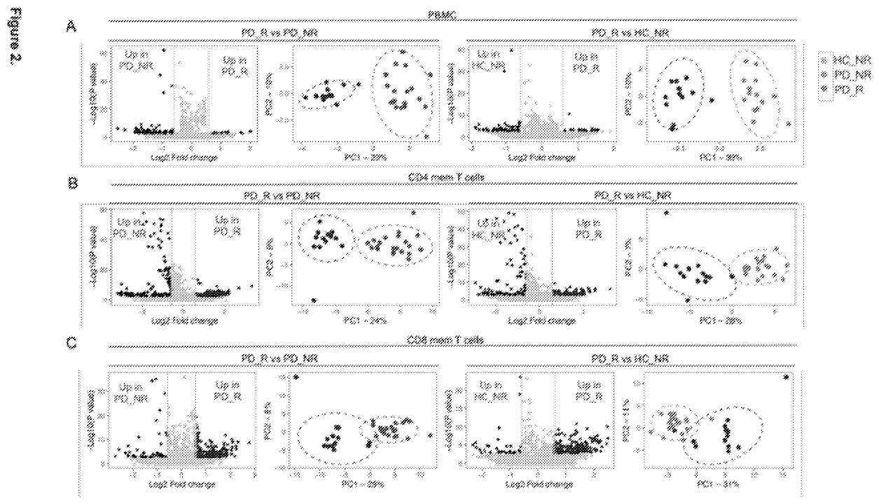

Next, the inventors compared the gene expression profiles of PD_R to HC_NR and to PD_NR subjects. The inventors observed a large increase in the number of differentially expressed genes in comparisons of each cell type (PBMC, CD4 and CD8 memory T cells; Table 1). The total number of differentially expressed genes for PBMC between PD_R versus PD_NR and PD_R versus HC_NR was 90 and 65, respectively (FIG. 2A). Scrutiny of these genes did not reveal any functional enrichment for specific patterns or pathways (Table 3).

In contrast, CD4 and CD8 memory T cells exhibited an intriguing gene signature with an approximately ˜2.5-4-fold increase in the number of differentially expressed genes between the PD_R and PD_NR groups and between PD_R and HC_NR. PD_R to PD_NR comparison revealed 304 DE genes for CD4 (136 down-regulated and 168 up-regulated; FIG. 2B), and 333 DE genes for CD8 (49 down-regulated and 284 up-regulated, FIG. 2C, Table 1). Similarly, comparing PD_R to HC_NR, revealed 172 DE genes for CD4 (91 down-regulated and 81 up-regulated, FIG. 2B), and 227 DE genes for CD8 (35 down-regulated and 192 up-regulated; FIG. 2C and Table 1). As expected, based on the DE genes, the disease groups PD_R, HC_NR, and PD_NR formed distinct clusters (FIG. 2). There was substantial overlap of DE genes between PD_R vs PD_NR and PD_R vs HC_NR within each cell type, but minimal to no overlap of DE genes across different cell types (Table 3).

PRKN and LRRK2 genes were differentially expressed in CD4 and CD8 memory T cells with both genes down-regulated in CD4 and up-regulated in CD8 memory T cells in PD_R compared to PD_NR and HC_NR respectively (PRKN is up in PD_R vs. PD_NR: LRRK2 is up in PD_R vs HC_NR) indicating that the two cell types play distinct roles in PD-associated T cell autoimmunity. In addition to PRKN, the inventors identified differentially expressed genes including as TFEB and UBAPIL in CD4 memory T cells.

### Example 4: Enrichment of PD Gene Signature in CD4 and CD8 Memory

### T Cells

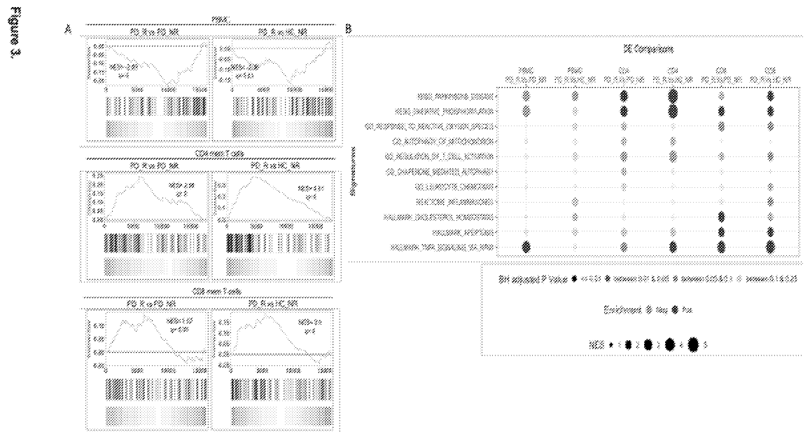

To further characterize the genes differentially expressed in PD_R, HC_NR, and PD_NR, the inventors performed gene set enrichment analysis (GSEA) (Subramanian et al., 2005). To check the enrichment of PD associated gene signature in the differentially expressed genes between PD_R vs. HC_NR and PD_R vs. PD_NR, the DE genes were ranked and compared to an existing gene set “KEGG PARKINSONS DISEASE” that was downloaded from MSigDB in GMT format (Liberzon et al., 2011). As shown in FIG. 3, a significant enrichment of PD associated genes in PD_R was observed in CD4 and CD8 memory T cells. However, no such enrichment was observed in PBMCs (FIG. 3A).

The inventors next examined the enrichment of several pathways implicated in PD, including oxidative phosphorylation (Shoffner et al., 1991), oxidative stress (Blesa et al., 2015; Dias et al., 2013; Hemmati-Dinarvand et al., 2019; Hwang, 2013; Jenner, 2003), macroautophagy and chaperone-mediated autophagy (Hou et al., 2020; Lynch-Day et al., 2012; Moors et al., 2017; Wang et al., 2016; Zhang et al., 2012), cholesterol signaling (Jin et al., 2019; Vance, 2012), inflammation (Stojkovska et al., 2015), and TNF signaling (Leal et al., 2013). Interestingly, chemotaxis, apoptosis, cholesterol biosynthesis and inflammation were significantly enriched in CD8 memory T cells and oxidative stress, autophagy of mitochondria and chaperone mediated autophagy were enriched in CD4 memory T cells. Other pathways, such as oxidative phosphorylation and TNF signaling were enriched in both memory T cell subsets (FIG. 3B).

The results suggest that classifying the PD subjects based on their α-syn T cell reactivity and separately examining memory CD4 and CD8 T cell subsets can detect PD associated gene signatures and identify PD relevant pathways (FIG. 3A-B). It further suggests that peripheral memory T cell subsets might offer an opportunity to dissect the molecular mechanisms associated with PD pathogenesis, and is consistent with the notion that memory T cells may play a significant role in PD pathogenesis.

### Example 5: Identification of Cell Surface and Secreted Protein Targets

Because cell surface expressed or secreted targets are amenable to modulation by monoclonal antibody therapy, the inventors were interested in identifying which of the differentially expressed genes encode surface expressed or secreted products that could be targeted in PD. The inventors performed surfaceome and secretome analysis on the differentially expressed genes between PD_R vs HC_NR and PD_R vs PD_NR in all cell types. For surfaceome analysis, three databases of surface expressing targets (Ashburner et al., 2000; Bausch-Fluck et al., 2018; Bausch-Fluck et al., 2015) were combined and a reference master list of targets that appeared in two out of three databases was generated that comprised of total 1168 targets. For secretome analysis, a reported human secretome database that comprised of 8575 targets was referred (Vathipadiekal et al., 2015). Combining surfaceome and secretome, the inventors identified 133 and 76 targets that were either secretory and/or surface expressed in PD_R vs PD_NR, and PD_R vs HC_NR, respectively, in the CD4 memory T cell subset. The inventors identified 140 and 100 targets in PD_R vs PD_NR, and PD_R vs HC_NR, respectively, in the CD8 memory T cell subset (Table 4).

The inventors further analyzed the dataset by annotating the ˜900 DE genes of Tables 3 and 4 using The Human Protein Atlas and Entrez Genome to assign cellular localization and known function(s). Next, the inventors chose to target genes that were either predicted to be membrane-bound or secreted from the cell.

The inventors identified 33 candidate genes that appear both in PD_NR and HC_NR comparisons with PD_R responders for each T cell type (CD4 and CD8). CD4 upregulate membrane protein candidates include LSMEM1, AIG1, APOL1, ABCD2, and CELSR2. CD4 upregulated secreted protein candidates include LEAP2, GDF11, LYPD8. CD8 upregulated membrane protein candidates include CALCRL, NTSR1, AC007040.2, OR1L8, and CCR1. CD8 upregulated secreted protein candidates include CFP, TNFSF13B, ADM5, LYZ, and LGALS3BP. LM07 is a membrane protein that exhibits upregulation in both CD4 and CD8 T cells. CD4 downregulate membrane protein candidates include RNF152, KCNH4, ABCC3, FFAR3, and CD300LB. CD4 downregulate secreted protein candidates include COL16A1, CPB2, IL22, IGFBP6, and ACAN. CD8 downregulate membrane protein candidates include KCNQ4, PAQR4, VAMP4, and CNIH2.

### Example 6: Validation of Potential Genes of Interest

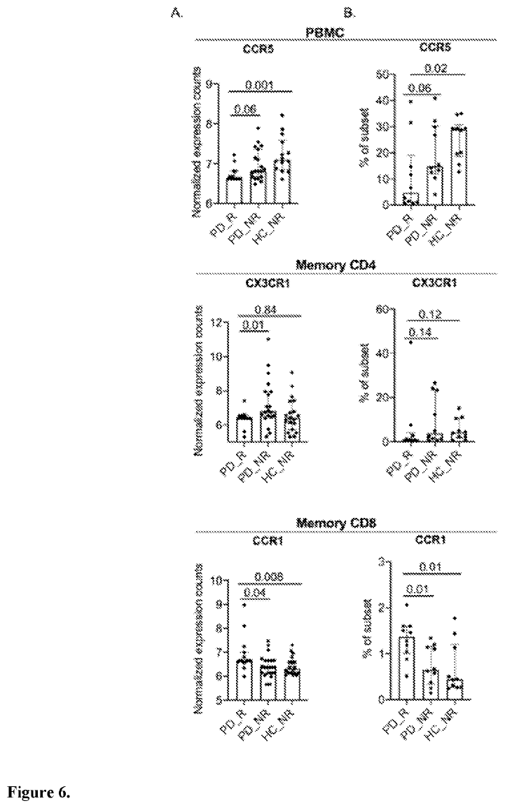

The inventors then selected specific DE genes for validation by flow cytometry based on the availability of commercially available antibodies. Specifically, the inventors validated one DE gene in each cell subset (CCR5 in PBMC; CX3CR1 in memory CD4 subset and CCR1 in memory CD8 subset) at the protein level. The normalized expression count of the genes that were validated is represented in FIG. 6A. The protein expression profile of the selected genes largely matched to the gene expression pattern observed by RNAseq analysis (FIG. 6B). For example, PBMCs of HC_NR displayed significantly higher expression of CCR5 than PD_R, the CD4 memory subset of PD_NR had higher expression of CX3CR1 than PD_R, and the CD8 memory subset of PD_R had significantly higher expression of CCR1 than PD_NR and HC_NR. Similar trends were observed at the transcriptional and protein levels.

### Example 7

In this disclosure, the inventors show that memory T cells of PD subjects with detectable α-syn responses possess specific mRNA signatures. These signatures are associated with novel genes targets for neurological diseases. The specific genes and pathways identified that show a significant enrichment of transcriptomic signatures previously associated with PD include oxidative stress, oxidative phosphorylation, autophagy of mitochondria, chaperone-mediated autophagy, cholesterol metabolism, and inflammation. These molecular pathways and the associated genes are known to be dysregulated in PD and are widely thought to accelerate the progression of disease. For instance, dysfunctional autophagic machinery leads to the accumulation of α-syn (Martinez-Vicente et al., 2008) and defective mitochondria (Lee et al., 2012) which in turn can lead to formation of α-syn aggregates or impair energy metabolism and cause oxidative stress. Moreover, the accumulated and misfolded α-syn, a protein normally involved in the regulation of synaptic vesicle exocytosis (Somayaji et al., 2020), causes degeneration of SNpc DA neurons, impairs synapse function (Chung et al., 2009; Ihara et al., 2007; Kahle et al., 2000; Sulzer and Edwards, 2019; Yavich et al., 2006) and affects respiration, morphology, and turnover of mitochondria (Chinta et al., 2010; Choubey et al., 2011; Cole et al., 2008; Devi et al., 2008; Li et al., 2007; Martin et al., 2006; Parihar et al., 2008, 2009), which may be related to display of mitochondrial-derived antigens in PD (Matheoud et al., 2019; McLelland et al., 2014). Additionally, cholesterol metabolism has also been linked to PD (Huang et al., 2019) via a potential role in synaptogenesis. The interplay of implicated pathways suggests that a cascade of several molecular events takes place, resulting in progressive neurodegeneration.

The inventors observed enrichment of reactive inflammasomes in CD8 memory T cell subset of PD responders, but not in their CD4 memory T cell subset, suggesting that PD associated inflammatory signature is cell type specific. The inventors focused on the signatures associated with CD4 and CD8 memory T cells. The focus on T cells is prompted and supported by several reports that imply a T cell-associated inflammatory process (Lindestam Arlehamn et al., 2020; Seo et al., 2020) within the PD prodromal phase and disease progression as well as in animal models (Matheoud et al., 2019). Specific transcriptomic signatures associated with CD4 and CD8 memory T cell compartments have been described in several other pathologies (Burel et al., 2018; Grifoni et al., 2018; Hyrcza et al., 2007; Patil et al., 2018; Tian et al., 2019a; Tian et al., 2019b), including autoimmunity (Hong et al., 2020; Lyons et al., 2010; McKinney et al., 2010); this is the first report of such signatures associated with memory T cells in neurodegenerative disease. A key element in this study was to focus on the transcriptional profile of specific purified memory CD4 and CD8 T cell subsets. Should this important aspect not have been considered, most of the differentially expressed genes and associated signatures would have been missed, as exemplified by the fact that very few differentially expressed genes were detected when whole PBMCs were considered.

As recently shown for monocytes, there can be a striking effect of sex on gene expression (Carlisle et al., 2021). The DE genes detected in this study did not suggest sex-specific differences and there was an equal distribution of males and females in the PD-R and PD-NR cohort.

Transcriptional signatures associated with PD have been reported by several groups based on analysis of samples of neural origin that includes astrocytes, neurons, and brain tissue including substantia nigra (Booth et al., 2019; Keo et al., 2020; Lang et al., 2019; Nido et al., 2020; Sandor et al., 2017). Here, the inventors studied the signatures of T cells isolated from peripheral blood, rather than the CNS, because of the difficulty of accessing the CNS, and importantly, because of the lack of availability of sufficient numbers of T cells available to study in CNS fluids from PD donors and in particular from healthy control subjects (Ransohoff et al., 2003). While future studies might further investigate T cells isolated directly from the CNS, it is known that infiltrating T cells recirculate between the blood and the CNS (Ransohoff et al., 2003; Shechter et al., 2013). To that end, the inventors detected multiple differences in chemokine receptor expression between the PD_R group compared to PD_NR and/or HC_NR. This included reduced CCR5 in PD_R PBMC, as well as a reduction in CX3CR1 signal in PD_R memory CD4 T cells. As for CX3CR1, its potential role in PD is mainly thought to be mediated through microglia (Angelopoulou et al., 2020); however, the receptor has been shown to define T cell memory populations (Gerlach et al., 2016) which have implications in disease (Yamauchi et al., 2020). In terms of PD pathogenesis, the reduced amount of circulating CCR5 or CX3CR1 expressing T cells in PD individuals might indicate an increased accumulation of those cells in the brain parenchyma where they could contribute to local inflammation.

Some of the DE genes found in PBMCs and T cells are implicated in PD pathogenesis. This includes leucine-rich repeat kinase 2 (LRRK2). It has been noted that LRRK2 is far more highly expressed in immune cells than neurons, and is also linked to Crohn's disease, an inflammatory bowel disorder, a class of disorders associated with PD (Herrick and Tansey, 2021). LRRK2 expression in PBMCs may be related to regulation of peripheral Type 2 interferon response that lead to dopamine neurodegeneration (Kozina et al., 2018), and its overall expression in T cells and other immune cells can be increased by interferon. In these results, LRRK2 transcript is decreased in PD to levels that are 33% the amount in HC.

Additional genes associated with mechanisms implicated in PD pathogenesis are also differentially expressed in T cells from PD_R subjects, including septin 5 (Son et al., 2005), the GDNF receptor (Sandmark et al., 2018), monoamine oxidase S, aquaporin (Tamtaji et al., 2019), LAMP3 (Liu et al., 2011) which has also been associated with REM sleep disorder (a risk factor for PD (Mufti et al., 2021)), polo-like kinase 1 (Mbefo et al., 2010), and myeloperoxidase (Maki et al., 2019). Most of these genes have been found previously to be expressed in neurons, but here the inventors show for the first time DE of these genes in peripheral cells. Moreover, these and additional DE genes point to the possibility that initiating steps in some PD pathogenic pathways might occur in peripheral immune cells and contribute to multiple hits that lead to the loss of targeted neurons (Raj et al., 2014).

Another key element in this study was a focus on the transcriptional profile of PD subjects that were classified based on their T cell responsiveness to α-syn, which were taken as a proxy for subjects undergoing an ongoing inflammatory autoimmune process. This was a determinant aspect, and if this important aspect not have been considered, most of the differentially expressed genes and associated signatures would have been missed. The classification of subjects based on T cell reactivity of α-syn might be further refined by considering additional antigens other than α-syn that might be also involved in PD pathogenesis (Latorre et al., 2018; Lindestam Arlehamn et al., 2019; Lodygin et al., 2019).

Based on a recently published conceptual model to describe PD pathogenesis (Johnson et al., 2019), factors that contribute to neurodegeneration can be divided into three categories: triggers, facilitators and aggravators. The study design focused on diagnosed PD patients with established disease, and is therefore likely addressing factors that contribute in disease spread (facilitators) and promote the neurodegenerative process (aggravators). Future studies in at risk categories for PD such as REM sleep disorder cohorts might shed light on RNA signatures associated with disease triggers.

This data identifies specific genes that could be addressed by therapeutic and diagnostic interventions, including TFEB, PRKN, SNCA, PARK2 and LRRK2. In a diagnostic setting, detection of alterations in the expression of these genes could contribute to a molecularly-based diagnostic, while in the therapeutic setting, it is possible that early targeting of the same genes by inhibiting or activating their function could delay or terminate disease progression or prevent disease development during the prodromal phase. Supportive of this notion is consistent the observation that anti-TNF treatment (Peter et al., 2018) is associated with lower PD disease incidence.

### Materials and Methods

Parkinson's disease (PD) is a multi-stage neurodegenerative disorder with largely unknown etiology. Recent findings have identified PD-associated autoimmune features including roles for T cells. To further characterize the role of T cells in PD, the inventors performed RNA sequencing on PBMC and peripheral CD4 and CD8 memory T cell subsets derived from PD patients and age-matched healthy controls. When the groups were stratified by their T cell responsiveness to alpha-synuclein (□-syn) as a proxy for ongoing inflammatory autoimmune response, the study revealed a broad differential gene expression profile in memory T cell subsets and a specific PD associated gene signature.

The inventors identified a significant enrichment of transcriptomic signatures previously associated with PD, including for oxidative stress, phosphorylation, autophagy of mitochondria, cholesterol metabolism and inflammation, and the chemokine signaling proteins CX3CR1, CCR5 and CCR1. In addition, the inventors identified genes in these peripheral cells that have previously been shown to be involved in PD pathogenesis and expressed in neurons, such as LRRK2, LAMP3, and aquaporin. Together, these findings suggest that features of circulating T cells with α-syn-specific responses in PD patients provide insights into the interactive processes that occur during PD pathogenesis and suggest potential intervention targets.

### Study Subjects

For RNAseq, the inventors recruited a total of 56 individuals diagnosed with PD (n=36) and age-matched healthy subjects (n=20) in this study. The subjects were recruited from multiple sites: 32 subjects from Columbia University Medical Center (CUMC) (PD n=26 and HC n=6), 10 subjects from La Jolla Institute for Immunology (LJI) (PD n=4 and HC n=6), 8 subjects from University of California San Diego (UCSD) (PD n=4 and HC n=4), 3 subjects from Rush University Medical Center (RUMC) (PD n=1 and HC n=2), 3 subjects from University of Alabama at Birmingham (UAB) (PD n=1 and HC n=2). For validation cohort, the inventors analyzed 30 subjects: 20 PD and 10 HC. The subjects were recruited from multiple sites: 10 subjects from Columbia University Medical Center (CUMC) (PD n=10), 12 subjects from La Jolla Institute for Immunology (LJI) (PD n=2 and HC n=10), 8 subjects from University of Alabama at Birmingham (UAB) (PD n=8). The characteristics of the enrolled subjects are detailed in Table 2.

The cohorts were recruited by the clinical core at LJI, by the Parkinson and Other Movement Disorder Center at UCSD, the clinical practice of the UAB Movement Disorders Clinic, and the Movement Disorders Clinic at the department of Neurology at CUMC. PD patients were enrolled on the basis of the following criteria: moderate to advanced PD; 2 of: rest tremor, rigidity, and/or bradykinesia, PD diagnosis at age 45-80, dopaminergic medication benefit, and ability to provide informed consent. The exclusion criteria were atypical parkinsonism or other neurological disorders, history of cancer within past 3 years, autoimmune disease, and chronic immune modulatory therapy. Age matched HC were selected on the basis of age 45-85 and ability to provide written consent. Exclusion criteria were the same as for PD donors and in addition, the inventors excluded self-reported genetic factors. The HC were not screened for prodromal symptoms. The PD patients recruited at RUMC, UAB, CUMC, and UCSD (i.e. not at LJI) all fulfilled the UK Parkinson's Disease Society Brain Bank criteria for PD. Patients with 0 years since diagnosis describe patients that had donated within their first year of being diagnosed with Parkinson's disease.

### Peptides

Peptides were commercially synthesized as purified material (>95% by reverse phase HPLC) on a small scale (1 mg/ml) by A&A, LLC (San Diego). A total of 11 peptides of α-syn (Sulzer et al., 2017) were synthesized and then reconstituted in DMSO at a concentration of 40 mg/ml. The individual peptides were then pooled, lyophilized and reconstituted at a concentration of 3.6 mg/ml. The peptide pools were tested at a final concentration of 5 ug/ml.

### PBMC Isolation

Venous blood was collected in heparin or EDTA containing blood bags and PBMCs were isolated by density gradient centrifugation using Ficoll-Paque plus (GE #17144003). Whole blood was first spun at 1850 rpm for 15 mins with brakes off to remove plasma. The plasma depleted blood was then diluted with RPMI and 35 ml of blood was gently layered on tubes containing 15 ml Ficoll-Paque plus. The tubes were then centrifuged at 1850 rpm for 25 mins with brakes off. The cells at the interface were collected, washed with RPMI, counted and cryopreserved in 90% v/v FBS and 10% v/v DMSO and stored in liquid nitrogen.

### Cell Sorting

The cryopreserved PBMC were thawed and revived in prewarmed RPMI media supplemented with 5% human serum (Gemini Bio-Products, West Sacramento, CA), 1% Glutamax (Gibco, Waltham, MA), 1% penicillin/streptomycin (Omega Scientific, Tarzana, CA), and 50 U/ml Benzonase (Millipore Sigma, Burlington, MA). The cells were then counted using hemocytometer, washed with PBS and prepared for staining. The cells at a density of 1 million were first incubated at 4° C. with 10% FBS for 10 mins for blocking and then stained with a mixture of the following antibodies: APCef780 conjugated anti-CD4 (clone RPA-T4, eBiosciences), AF700 conjugated anti-CD3 (clone UCHT1, BD Pharmigen), BV650 conjugated anti-CD8a (clone RPA-T8, Biolegend), PECy7 conjugated anti-CD19 (clone HIB19, TONBO), APC conjugated anti-CD14 (clone 61D3, TONBO), PerCPCy5.5 conjugated anti-CCR7 (clone G043H7, Biolegend), PE conjugated anti-CD56 (eBiosciences), FITC conjugated anti-CD25 (clone M-A251, BD Pharmigen), eF450 conjugated anti-CD45RA (clone HI100, eBiosciences) and eF506 live dead aqua dye (eBiosciences) for 30 mins at 4° C. Cells were then washed twice and resuspended in 100 ul PBS for flow cytometric analysis and sorting. The cells were sorted using BD FACSAria- (BD Biosciences) into ice cold Trizol LS reagent (Thermo Fisher Scientific).

### Fluorospot Assay

PBMCs were thawed and stimulated for two weeks in vitro with α-syn pools. PHA was used as control. Cells were fed with 10 U/ml recombinant IL-2 at an interval of 4 days. After two weeks of culture, T cell responses to α-syn were measured by IFNγ, IL-5 and IL-10 Fluorospot assay. Plates (Mabtech, Nacka Strand, Sweden) were coated overnight at 4° C. with an antibody mixture of mouse anti-human IFNγ clone (clone 1-D1K), mouse anti human IL-5 (clone TRFK5), and mouse anti-human IL-10 (clone 9D7). Briefly, 100,000 cells were plated in each well of the pre-coated Immobilon-FL PVDF 96 well plates (Mabtech), stimulated with the respective antigen at the respective concentration of 5 μg/ml and incubated at 37° C. in a humidified CO2 incubator for 20-24 hrs. Cells stimulated with α-syn were also stimulated with 10 μg/ml PHA that served as a positive control. In order to assess nonspecific cytokine production, cells were also stimulated with DMSO at the corresponding concentration present in the peptide pools. All conditions were tested in triplicates. After incubation, cells were removed, plates were washed six times with 200 μl PBS/0.05% Tween 20 using an automated plate washer. After washing, 100 μl of an antibody mixture containing IFNγ (7-B6-1-FS-BAM), IL-5 (5A10-WASP), and IL-10 (12G8-biotin) prepared in PBS with 0.1% BSA was added to each well and plates were incubated for 2 hrs at room temperature. The plates were again washed six times as described above and incubated with diluted fluorophores (anti-BAM-490, anti-WASP-640, and SA-550) for 1 hr at room temperature. After incubation, the plates were again washed as described above and incubated with a fluorescence enhancer for 15 min. Finally, the plates were blotted dry and spots were counted by computer-assisted image analysis (AID iSpot, AID Diagnostica GMBH, Strassberg, Germany). The responses were considered positive if they met all three criteria (i) the net spot forming cells per 106 PBMC were ≥100 (ii) the stimulation index ≥2, and (iii) p≤0.05 by Student's t test or Poisson distribution test.

### Smart-Seq

PBMC, CD4 and CD8 memory T cells of PD and HC subjects were sorted and total RNA from ˜50,000 cells was extracted on a Qiacube using a miRNA easy kit (Qiagen) and quantified using bioanalyzer. Total RNA was amplified according to Smart Seq protocol (Picelli et al., 2014). cDNA was purified using AMPure XP beads. cDNA was used to prepare a standard barcoded sequencing library (Illumina). Samples were sequenced using an Illumina HiSeq2500 to obtain 50-bp single end reads. Samples that failed to be sequenced due to limited sample availability or failed the quality control were eliminated from further sequencing and analysis.

### RNA-Seq Analysis

The reads that passed Illumina filters were further filtered for reads aligning to tRNA, rRNA, adapter sequences, and spike-in controls. These reads were then aligned to GRCh38 reference genome and Gencode v27 annotations using STAR: v2.6.1 (Dobin et al., 2013). DUST scores were calculated with PRINSEQ Lite (v 0.20.3) (Schmieder and Edwards, 2011) and low-complexity reads (DUST >4) were removed from the BAM files. The alignment results were parsed via the SAMtools (Li et al., 2009) to generate SAM files. Read counts to each genomic feature were obtained with featureCounts (v 1.6.5) (Liao et al., 2014) with default options. After removing absent features (zero counts in all samples), the raw counts were then imported to R/Bioconductor package DESeq2 (v 1.24.0) (Love et al., 2014) to identify differentially expressed genes among samples. Known batch conditions cohort and mapping run id were used in the design formula to correct for unwanted variation in the data. P-values for differential expression were calculated using the Wald test for differences between the base means of two conditions. These P-values are then adjusted for multiple test correction using Benjamini Hochberg algorithm (Benjamini and Hochberg, 1995). The inventors considered genes differentially expressed between two groups of samples when the DESeq2 analysis resulted in an adjusted P-value of <0.05 and the difference in gene expression was 1.5-fold. The sequences used in this article have been submitted to the Gene Expression Omnibus under accession number GSE174473 (http://www.ncbi.nlm.nih.gov/geo/).

### GSEA

Gene set enrichment analysis was done using the “GseaPreranked” method with “classic” scoring scheme and other default settings. The geneset KEGG PARKINSONS DISEASE was downloaded from MSigDB in GMT format (https://www.gseamsigdb.org/gsea/msigdb/cards/KEGG_PARKINSONS_DISEASE). Rank files for the DE comparisons of interest were generated by assigning a rank of −log 10(p Value) to protein coding genes with log 2FoldChange greater than zero and log 10(p Value) to genes with log 2 FoldChange less than zero. The GSEA figures were generated using ggplot2 package in R with gene ranks as the x-axis and enrichment score as the y-axis. The heatmap bar was generated using ggplot with genes ordered by their rank on x-axis and 1 as y-axis. Log 2FoldChange values were used as the aes color option. scale_colour_gradient2 function was used with a midpoint=0 and other default options.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

The disclosures illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

Angelopoulou, E., Paudel, Y. N., Shaikh, M. F., and Piperi, C. (2020). Fractalkine (CX3CL1) signaling and neuroinflammation in Parkinson's disease: Potential clinical and therapeutic implications. Pharmacol Res 158, 104930. Archibald, N., Miller, N., and Rochester, L. (2013). Neurorehabilitation in Parkinson disease. Handb Clin Neurol 110, 435-442.
Ashburner, M., Ball, C. A., Blake, J. A., Botstein, D., Butler, H., Cherry, J. M., Davis, A. P., Dolinski, K., Dwight, S. S., Eppig, J. T., et al. (2000). Gene ontology: tool for the unification of biology. The Gene Ontology Consortium. Nat Genet 25, 25-29.
Bausch-Fluck, D., Goldmann, U., Muller, S., van Oostrum, M., Muller, M., Schubert, O. T., and Wollscheid, B. (2018). The in silico human surfaceome. Proc Natl Acad Sci USA 115, E10988-E10997.
Bausch-Fluck, D., Hofmann, A., Bock, T., Frei, A. P., Cerciello, F., Jacobs, A., Moest, H., Omasits, U., Gundry, R. L., Yoon, C., et al. (2015). A mass spectrometric-derived cell surface protein atlas. PLoS One 10, e0121314.
Benjamini, Y., and Hochberg, Y. (1995). Controlling the False Discovery Rate: A Practical and Powerful Approach to Multiple Testing. Journal of the Royal Statistical Society Series B (Methodological) 57, 289-300.
Blesa, J., Trigo-Damas, I., Quiroga-Varela, A., and Jackson-Lewis, V. R. (2015). Oxidative stress and Parkinson's disease. Front Neuroanat 9, 91.
Booth, H. D. E., Wessely, F., Connor-Robson, N., Rinaldi, F., Vowles, J., Browne, C., Evetts, S. G., Hu, M. T., Cowley, S. A., Webber, C., et al. (2019). RNA sequencing reveals MMP2 and TGFB1 downregulation in LRRK2 G2019S Parkinson's iPSC-derived astrocytes. Neurobiol Dis 129, 56-66.
Burel, J. G., Lindestam Arlehamn, C. S., Khan, N., Seumois, G., Greenbaum, J. A., Taplitz, R., Gilman, R. H., Saito, M., Vijayanand, P., Sette, A., et al. (2018). Transcriptomic Analysis of CD4(+) T Cells Reveals Novel Immune Signatures of Latent Tuberculosis. J Immunol 200, 3283-3290.
Carlisle, S. M., Qin, H., Hendrickson, R. C., Muwanguzi, J. E., Lefkowitz, E. J., Kennedy, R. E., Yan, Z., Yacoubian, T. A., Benveniste, E. N., West, A. B., et al. (2021). Sex-based differences in the activation of peripheral blood monocytes in early Parkinson disease. NPJ Parkinsons Dis 7, 36.
Chinta, S. J., Mallajosyula, J. K., Rane, A., and Andersen, J. K. (2010). Mitochondrial alpha-synuclein accumulation impairs complex I function in dopaminergic neurons and results in increased mitophagy in vivo. Neurosci Lett 486, 235-239.
Choubey, V., Safiulina, D., Vaarmann, A., Cagalinec, M., Wareski, P., Kuum, M., Zharkovsky, A., and Kaasik, A. (2011). Mutant A53T alpha-synuclein induces neuronal death by increasing mitochondrial autophagy. J Biol Chem 286, 10814-10824.
Chung, C. Y., Koprich, J. B., Siddiqi, H., and Isacson, O. (2009). Dynamic changes in presynaptic and axonal transport proteins combined with striatal neuroinflammation precede dopaminergic neuronal loss in a rat model of AAV alpha-synucleinopathy. J Neurosci 29, 3365-3373.
Cole, N. B., Dieuliis, D., Leo, P., Mitchell, D. C., and Nussbaum, R. L. (2008). Mitochondrial translocation of alpha-synuclein is promoted by intracellular acidification. Exp Cell Res 314, 2076-2089.
Dachsel, J. C., and Farrer, M. J. (2010). LRRK2 and Parkinson disease. Arch Neurol 67, 542-547.
Dawson, T. M., and Dawson, V. L. (2010). The role of parkin in familial and sporadic Parkinson's disease. Mov Disord 25 Suppl 1, S32-39.
Decressac, M., and Bjorklund, A. (2013). TFEB: Pathogenic role and therapeutic target in Parkinson disease. Autophagy 9, 1244-1246.
Devi, L., Raghavendran, V., Prabhu, B. M., Avadhani, N. G., and Anandatheerthavarada, H. K. (2008). Mitochondrial import and accumulation of alpha-synuclein impair complex I in human dopaminergic neuronal cultures and Parkinson disease brain. J Biol Chem 283, 9089-9100.
Di Maio, R., Hoffman, E. K., Rocha, E. M., Keeney, M. T., Sanders, L. H., De Miranda, B. R., Zharikov, A., Van Laar, A., Stepan, A. F., Lanz, T. A., et al. (2018). LRRK2 activation in idiopathic Parkinson's disease. Sci Transl Med 10.
Dias, V., Junn, E., and Mouradian, M. M. (2013). The role of oxidative stress in Parkinson's disease. J Parkinsons Dis 3, 461-491.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Fahn, S., and Sulzer, D. (2004). Neurodegeneration and neuroprotection in Parkinson disease. NeuroRx 1, 139-154.
Gerlach, C., Moseman, E. A., Loughhead, S. M., Alvarez, D., Zwijnenburg, A. J., Waanders, L., Garg, R., de la Torre, J. C., and von Andrian, U. H. (2016). The Chemokine Receptor CX3CR1 Defines Three Antigen-Experienced CD8 T Cell Subsets with Distinct Roles in Immune Surveillance and Homeostasis. Immunity 45, 1270-1284.
Grifoni, A., Costa-Ramos, P., Pham, J., Tian, Y., Rosales, S. L., Seumois, G., Sidney, J., de Silva, A. D., Premkumar, L., Collins, M. H., et al. (2018). Cutting Edge: Transcriptional Profiling Reveals Multifunctional and Cytotoxic Antiviral Responses of Zika Virus-Specific CD8(+) T Cells. J Immunol 201, 3487-3491.

Hemmati-Dinarvand, M., Saedi, S., Valilo, M., Kalantary-Charvadeh, A., Alizadeh Sani, M., Kargar, R., Safari, H., and Samadi, N. (2019). Oxidative stress and Parkinson's disease: conflict of oxidant-antioxidant systems. Neurosci Lett 709, 134296.
Herrick, M. K., and Tansey, M. G. (2021). Is LRRK2 the missing link between inflammatory bowel disease and Parkinson's disease? NPJ Parkinsons Dis 7, 26.
Hong, X., Meng, S., Tang, D., Wang, T., Ding, L., Yu, H., Li, H., Liu, D., Dai, Y., and Yang, M. (2020). Single-Cell RNA Sequencing Reveals the Expansion of Cytotoxic CD4(+) T Lymphocytes and a Landscape of Immune Cells in Primary Sjogren's Syndrome. Front Immunol 11, 594658.
Hou, X., Watzlawik, J. O., Fiesel, F. C., and Springer, W. (2020). Autophagy in Parkinson's Disease. J Mol Biol 432, 2651-2672.
Huang, X., Sterling, N. W., Du, G., Sun, D., Stetter, C., Kong, L., Zhu, Y., Neighbors, J., Lewis, M. M., Chen, H., et al. (2019). Brain cholesterol metabolism and Parkinson's disease. Mov Disord 34, 386-395.
Hwang, O. (2013). Role of oxidative stress in Parkinson's disease. Exp Neurobiol 22, 11-17.
Hyrcza, M. D., Kovacs, C., Loutfy, M., Halpenny, R., Heisler, L., Yang, S., Wilkins, O., Ostrowski, M., and Der, S. D. (2007). Distinct transcriptional profiles in ex vivo CD4+ and CD8+ T cells are established early in human immunodeficiency virus type 1 infection and are characterized by a chronic interferon response as well as extensive transcriptional changes in CD8+ T cells. J Virol 81, 3477-3486.
Ihara, M., Yamasaki, N., Hagiwara, A., Tanigaki, A., Kitano, A., Hikawa, R., Tomimoto, H., Noda, M., Takanashi, M., Mori, H., et al. (2007). September 4, a component of presynaptic scaffold and Lewy bodies, is required for the suppression of alpha-synuclein neurotoxicity. Neuron 53, 519-533.
Jenner, P. (2003). Oxidative stress in Parkinson's disease. Ann Neurol 53 Suppl 3, S26-36; discussion S36-28.
Jin, U., Park, S. J., and Park, S. M. (2019). Cholesterol Metabolism in the Brain and Its Association with Parkinson's Disease. Exp Neurobiol 28, 554-567.
Johnson, M. E., Stecher, B., Labrie, V., Brundin, L., and Brundin, P. (2019). Triggers, Facilitators, and Aggravators: Redefining Parkinson's Disease Pathogenesis. Trends Neurosci 42, 4-13.
Kahle, P. J., Neumann, M., Ozmen, L., Muller, V., Jacobsen, H., Schindzielorz, A., Okochi, M., Leimer, U., van Der Putten, H., Probst, A., et al. (2000). Subcellular localization of wild-type and Parkinson's disease-associated mutant alpha-synuclein in human and transgenic mouse brain. J Neurosci 20, 6365-6373.
Keo, A., Mahfouz, A., Ingrassia, A. M. T., Meneboo, J. P., Villenet, C., Mutez, E., Comptdaer, T., Lelieveldt, B. P. F., Figeac, M., Chartier-Harlin, M. C., et al. (2020). Transcriptomic signatures of brain regional vulnerability to Parkinson's disease. Commun Biol 3, 101.
Kozina, E., Sadasivan, S., Jiao, Y., Dou, Y., Ma, Z., Tan, H., Kodali, K., Shaw, T., Peng, J., and Smeyne, R. J. (2018). Mutant LRRK2 mediates peripheral and central immune responses leading to neurodegeneration in vivo. Brain 141, 1753-1769.
Lang, C., Campbell, K. R., Ryan, B. J., Carling, P., Attar, M., Vowles, J., Perestenko, O. V., Bowden, R., Baig, F., Kasten, M., et al. (2019). Single-Cell Sequencing of iPSC-Dopamine Neurons Reconstructs Disease Progression and Identifies HDAC4 as a Regulator of Parkinson Cell Phenotypes. Cell Stem Cell 24, 93-106 e106.
Latorre, D., Kallweit, U., Armentani, E., Foglierini, M., Mele, F., Cassotta, A., Jovic, S., Jarrossay, D., Mathis, J., Zellini, F., et al. (2018). T cells in patients with narcolepsy target self-antigens of hypocretin neurons. Nature 562, 63-68.
Leal, M. C., Casabona, J. C., Puntel, M., and Pitossi, F. J. (2013). Interleukin-1beta and tumor necrosis factor-alpha: reliable targets for protective therapies in Parkinson's Disease? Front Cell Neurosci 7, 53.
Lee, J., Giordano, S., and Zhang, J. (2012). Autophagy, mitochondria and oxidative stress: cross-talk and redox signalling. Biochem J 441, 523-540.
Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.
Li, J. Q., Tan, L., and Yu, J. T. (2014). The role of the LRRK2 gene in Parkinsonism. Mol Neurodegener 9, 47.
Li, W. W., Yang, R., Guo, J. C., Ren, H. M., Zha, X. L., Cheng, J. S., and Cai, D. F. (2007). Localization of alpha-synuclein to mitochondria within midbrain of mice. Neuroreport 18, 1543-1546.
Liao, Y., Smyth, G. K., and Shi, W. (2014). featureCounts: an efficient general purpose program for assigning sequence reads to genomic features. Bioinformatics 30, 923-930.
Liberzon, A., Subramanian, A., Pinchback, R., Thorvaldsdottir, H., Tamayo, P., and Mesirov, J. P. (2011). Molecular signatures database (MSigDB) 3.0. Bioinformatics 27, 1739-1740.
Lindestam Arlehamn, C. S., Dhanwani, R., Pham, J., Kuan, R., Frazier, A., Rezende Dutra, J., Phillips, E., Mallal, S., Roederer, M., Marder, K. S., et al. (2020). alpha-Synuclein-specific T cell reactivity is associated with preclinical and early Parkinson's disease. Nature communications 11, 1875.
Lindestam Arlehamn, C. S., Pham, J., Alcalay, R. N., Frazier, A., Shorr, E., Carpenter, C., Sidney, J., Dhanwani, R., Agin-Liebes, J., Garretti, F., et al. (2019). Widespread Tau-Specific CD4 T Cell Reactivity in the General Population. J Immunol 203, 84-92.
Liu, X., Cheng, R., Verbitsky, M., Kisselev, S., Browne, A., Mejia-Sanatana, H., Louis, E. D., Cote, L. J., Andrews, H., Waters, C., et al. (2011). Genome-wide association study identifies candidate genes for Parkinson's disease in an Ashkenazi Jewish population. BMC Med Genet 12, 104.
Lodygin, D., Hermann, M., Schweingruber, N., Flugel-Koch, C., Watanabe, T., Schlosser, C., Merlini, A., Korner, H., Chang, H. F., Fischer, H. J., et al. (2019). beta-Synuclein-reactive T cells induce autoimmune CNS grey matter degeneration. Nature 566, 503-508.
Love, M. I., Huber, W., and Anders, S. (2014). Moderated estimation of fold change and dispersion for RNA-seq data with DESeq2. Genome Biol 15, 550.
Lynch-Day, M. A., Mao, K., Wang, K., Zhao, M., and Klionsky, D. J. (2012). The role of autophagy in Parkinson's disease. Cold Spring Harb Perspect Med 2, a009357.
Lyons, P. A., McKinney, E. F., Rayner, T. F., Hatton, A., Woffendin, H. B., Koukoulaki, M., Freeman, T. C., Jayne, DR., Chaudhry, A. N., and Smith, K. G. (2010). Novel expression signatures identified by transcriptional analysis of separated leucocyte subsets in systemic lupus erythematosus and vasculitis. Annals of the rheumatic diseases 69, 1208-1213.
Maki, R. A., Holzer, M., Motamedchaboki, K., Malle, E., Masliah, E., Marsche, G., and Reynolds, W. F. (2019). Human myeloperoxidase (hMPO) is expressed in neurons in the substantia nigra in Parkinson's disease and in the hMPO-alpha-synuclein-A53T mouse model, correlating with increased nitration and aggregation of alpha-synuclein and exacerbation of motor impairment. Free Radic Biol Med 141, 115-140.
Marras, C., Beck, J. C., Bower, J. H., Roberts, E., Ritz, B., Ross, G. W., Abbott, R. D., Savica, R., Van Den Eeden, S. K., Willis, A. W., et al. (2018). Prevalence of Parkinson's disease across North America. NPJ Parkinsons Dis 4, 21.
Martin, L. J., Pan, Y., Price, A. C., Sterling, W., Copeland, N. G., Jenkins, N. A., Price, D. L., and Lee, M. K. (2006). Parkinson's disease alpha-synuclein transgenic mice develop neuronal mitochondrial degeneration and cell death. J Neurosci 26, 41-50.
Martinez-Vicente, M., Talloczy, Z., Kaushik, S., Massey, A. C., Mazzulli, J., Mosharov, E. V., Hodara, R., Fredenburg, R., Wu, D. C., Follenzi, A., et al. (2008). Dopamine-modified alpha-synuclein blocks chaperone-mediated autophagy. J Clin Invest 118, 777-788.
Mata, I. F., Wedemeyer, W. J., Farrer, M. J., Taylor, J. P., and Gallo, K. A. (2006). LRRK2 in Parkinson's disease: protein domains and functional insights. Trends Neurosci 29, 286-293.
Matheoud, D., Cannon, T., Voisin, A., Penttinen, A. M., Ramet, L., Fahmy, A. M., Ducrot, C., Laplante, A., Bourque, M. J., Zhu, L., et al. (2019). Intestinal infection triggers Parkinson's disease-like symptoms in Pink1(−/−) mice. Nature 571, 565-569.
Mbefo, M. K., Paleologou, K. E., Boucharaba, A., Oueslati, A., Schell, H., Fournier, M., Olschewski, D., Yin, G., Zweckstetter, M., Masliah, E., et al. (2010). Phosphorylation of synucleins by members of the Polo-like kinase family. J Biol Chem 285, 2807-2822.
McGeer, P. L., Itagaki, S., Boyes, B. E., and McGeer, E. G. (1988). Reactive microglia are positive for HLA-DR in the substantia nigra of Parkinson's and Alzheimer's disease brains. Neurology 38, 1285-1291.
McKinney, E. F., Lyons, P. A., Carr, E. J., Hollis, J. L., Jayne, DR., Willcocks, L. C., Koukoulaki, M., Brazma, A., Jovanovic, V., Kemeny, D. M., et al. (2010). A CD8+ T cell transcription signature predicts prognosis in autoimmune disease. Nat Med 16, 586-591, 581p following 591.
McLelland, G. L., Soubannier, V., Chen, C. X., McBride, H. M., and Fon, E. A. (2014). Parkin and PINK 1 function in a vesicular trafficking pathway regulating mitochondrial quality control. EMBO J 33, 282-295.
Mondal, S., Rangasamy, S. B., Roy, A., Dasarathy, S., Kordower, J. H., and Pahan, K. (2019). Low-Dose Maraviroc, an Antiretroviral Drug, Attenuates the Infiltration of T Cells into the Central Nervous System and Protects the Nigrostriatum in Hemiparkinsonian Monkeys. J Immunol.
Moors, T. E., Hoozemans, J. J., Ingrassia, A., Beccari, T., Parnetti, L., Chartier-Harlin, M. C., and van de Berg, W. D. (2017). Therapeutic potential of autophagy-enhancing agents in Parkinson's disease. Mol Neurodegener 12, 11.
Mufti, K., Yu, E., Rudakou, U., Krohn, L., Ruskey, J. A., Asayesh, F., Laurent, S. B., Spiegelman, D., Arnulf, I., Hu, M. T. M., et al. (2021). Novel Associations of BST1 and LAMP3 With REM Sleep Behavior Disorder. Neurology 96, e1402-e1412.
Nido, G. S., Dick, F., Toker, L., Petersen, K., Alves, G., Tysnes, O. B., Jonassen, I., Haugarvoll, K., and Tzoulis, C. (2020). Common gene expression signatures in Parkinson's disease are driven by changes in cell composition. Acta Neuropathol Commun 8, 55.
Parihar, M. S., Parihar, A., Fujita, M., Hashimoto, M., and Ghafourifar, P. (2008). Mitochondrial association of alpha-synuclein causes oxidative stress. Cell Mol Life Sci 65, 1272-1284.
Parihar, M. S., Parihar, A., Fujita, M., Hashimoto, M., and Ghafourifar, P. (2009). Alpha-synuclein overexpression and aggregation exacerbates impairment of mitochondrial functions by augmenting oxidative stress in human neuroblastoma cells. Int J Biochem Cell Biol 41, 2015-2024.
Patil, V. S., Madrigal, A., Schmiedel, B. J., Clarke, J., O'Rourke, P., de Silva, A. D., Harris, E., Peters, B., Seumois, G., Weiskopf, D., et al. (2018). Precursors of human CD4(+) cytotoxic T lymphocytes identified by single-cell transcriptome analysis. Sci Immunol 3.
Peter, I., Dubinsky, M., Bressman, S., Park, A., Lu, C., Chen, N., and Wang, A. (2018). Anti-Tumor Necrosis Factor Therapy and Incidence of Parkinson Disease Among Patients With Inflammatory Bowel Disease. JAMA Neurol 75, 939-946.
Picelli, S., Faridani, O. R., Bjorklund, A. K., Winberg, G., Sagasser, S., and Sandberg, R. (2014). Full-length RNA-seq from single cells using Smart-seq2. Nature protocols 9, 171-181.
Postuma, R. B., and Berg, D. (2016). Advances in markers of prodromal Parkinson disease. Nat Rev Neurol 12, 622-634.
Raj, T., Rothamel, K., Mostafavi, S., Ye, C., Lee, M. N., Replogle, J. M., Feng, T., Lee, M., Asinovski, N., Frohlich, I., et al. (2014). Polarization of the effects of autoimmune and neurodegenerative risk alleles in leukocytes. Science 344, 519-523.
Ransohoff, R. M., Kivisakk, P., and Kidd, G. (2003). Three or more routes for leukocyte migration into the central nervous system. Nat Rev Immunol 3, 569-581.
Rui, Q., Ni, H., Li, D., Gao, R., and Chen, G. (2018). The Role of LRRK2 in Neurodegeneration of Parkinson Disease. Curr Neuropharmacol 16, 1348-1357.
Sandmark, J., Dahl, G., Oster, L., Xu, B., Johansson, P., Akerud, T., Aagaard, A., Davidsson, P., Bigalke, J. M., Winzell, M. S., et al. (2018). Structure and biophysical characterization of the human full-length neurturin-GFRa2 complex: A role for heparan sulfate in signaling. J Biol Chem 293, 5492-5508.
Sandor, C., Robertson, P., Lang, C., Heger, A., Booth, H., Vowles, J., Witty, L., Bowden, R., Hu, M., Cowley, S. A., et al. (2017). Transcriptomic profiling of purified patient-derived dopamine neurons identifies convergent perturbations and therapeutics for Parkinson's disease. Hum Mol Genet 26, 552-566.
Schmieder, R., and Edwards, R. (2011). Quality control and preprocessing of metagenomic datasets. Bioinformatics 27, 863-864.
Seo, J., Park, J., Kim, K., Won, J., Yeo, H. G., Jin, Y. B., Koo, B. S., Lim, K. S., Jeong, K. J., Kang, P., et al. (2020). Chronic Infiltration of T Lymphocytes into the Brain in a Non-human Primate Model of Parkinson's Disease. Neuroscience 431, 73-85.
Settembre, C., Di Malta, C., Polito, V. A., Garcia Arencibia, M., Vetrini, F., Erdin, S., Erdin, S. U., Huynh, T., Medina, D., Colella, P., et al. (2011). TFEB links autophagy to lysosomal biogenesis. Science 332, 1429-1433.
Shechter, R., London, A., and Schwartz, M. (2013). Orchestrated leukocyte recruitment to immune-privileged sites: absolute barriers versus educational gates. Nat Rev Immunol 13, 206-218.
Shoffner, J. M., Watts, R. L., Juncos, J. L., Torroni, A., and Wallace, D. C. (1991). Mitochondrial oxidative phosphorylation defects in Parkinson's disease. Ann Neurol 30, 332-339.
Somayaji, M., Cataldi, S., Choi, S. J., Edwards, R. H., Mosharov, E. V., and Sulzer, D. (2020). A dual role for alpha-synuclein in facilitation and depression of dopamine release from substantia nigra neurons in vivo. Proc Natl Acad Sci USA 117, 32701-32710.
Son, J. H., Kawamata, H., Yoo, M. S., Kim, D. J., Lee, Y. K., Kim, S., Dawson, T. M., Zhang, H., Sulzer, D., Yang, L., et al. (2005). Neurotoxicity and behavioral deficits associated with Septin 5 accumulation in dopaminergic neurons. J Neurochem 94, 1040-1053.
Spillantini, M. G., Schmidt, M. L., Lee, V. M., Trojanowski, J. Q., Jakes, R., and Goedert, M. (1997). Alpha-synuclein in Lewy bodies. Nature 388, 839-840.
Stojkovska, I., Wagner, B. M., and Morrison, B. E. (2015). Parkinson's disease and enhanced inflammatory response. Exp Biol Med (Maywood) 240, 1387-1395.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Sulzer, D., Alcalay, R. N., Garretti, F., Cote, L., Kanter, E., Agin-Liebes, J., Liong, C., McMurtrey, C., Hildebrand, W. H., Mao, X., et al. (2017). T cells from patients with Parkinson's disease recognize alpha-synuclein peptides. Nature 546, 656-661.
Sulzer, D., and Edwards, R. H. (2019). The physiological role of alpha-synuclein and its relationship to Parkinson's Disease. J Neurochem 150, 475-486.
Tamtaji, O. R., Behnam, M., Pourattar, M. A., Jafarpour, H., and Asemi, Z. (2019). Aquaporin 4: A key player in Parkinson's disease. J Cell Physiol 234, 21471-21478.
Tian, Y., Babor, M., Lane, J., Seumois, G., Liang, S., Goonawardhana, N. D. S., De Silva, A. D., Phillips, E. J., Mallal, S. A., da Silva Antunes, R., et al. (2019a). Dengue-specific CD8+ T cell subsets display specialized transcriptomic and TCR profiles. J Clin Invest 129, 1727-1741.
Tian, Y., Seumois, G., De-Oliveira-Pinto, L. M., Mateus, J., Herrera-de la Mata, S., Kim, C., Hinz, D., Goonawardhana, N. D. S., de Silva, A. D., Premawansa, S., et al. (2019b). Molecular Signatures of Dengue Virus-Specific IL-10/IFN-gamma Co-producing CD4 T Cells and Their Association with Dengue Disease. Cell Rep 29, 4482-4495 e4484.
Torra, A., Parent, A., Cuadros, T., Rodriguez-Galvan, B., Ruiz-Bronchal, E., Ballabio, A., Bortolozzi, A., Vila, M., and Bove, J. (2018). Overexpression of TFEB Drives a Pleiotropic Neurotrophic Effect and Prevents Parkinson's Disease-Related Neurodegeneration. Mol Ther 26, 1552-1567.
Vance, J. E. (2012). Dysregulation of cholesterol balance in the brain: contribution to neurodegenerative diseases. Dis Model Mech 5, 746-755.
Vathipadiekal, V., Wang, V., Wei, W., Waldron, L., Drapkin, R., Gillette, M., Skates, S., and Birrer, M. (2015). Creation of a Human Secretome: A Novel Composite Library of Human Secreted Proteins: Validation Using Ovarian Cancer Gene Expression Data and a Virtual Secretome Array. Clin Cancer Res 21, 4960-4969.
von Coelln, R., Dawson, V. L., and Dawson, T. M. (2004). Parkin-associated Parkinson's disease. Cell Tissue Res 318, 175-184.
Wang, B., Abraham, N., Gao, G., and Yang, Q. (2016). Dysregulation of autophagy and mitochondrial function in Parkinson's disease. Transl Neurodegener 5, 19.
Yamauchi, T., Hoki, T., Oba, T., Saito, H., Attwood, K., Sabel, M. S., Chang, A. E., Odunsi, K., and Ito, F. (2020). CX3CR1-CD8+ T cells are critical in antitumor efficacy but functionally suppressed in the tumor microenvironment. JCI Insight 5.
Yavich, L., Jakala, P., and Tanila, H. (2006). Abnormal compartmentalization of norepinephrine in mouse dentate gyros in alpha-synuclein knockout and A30P transgenic mice. J Neurochem 99, 724-732.
Zhang, L., Dong, Y., Xu, X., and Xu, Z. (2012). The role of autophagy in Parkinson's disease. Neural Regen Res 7, 141-145.
Zhuang, X. X., Wang, S. F., Tan, Y., Song, J. X., Zhu, Z., Wang, Z. Y., Wu, M. Y., Cai, C. Z., Huang, Z. J., Tan, J. Q., et al. (2020). Pharmacological enhancement of TFEB-mediated autophagy alleviated neuronal death in oxidative stress-induced Parkinson's disease models. Cell Death Dis 11, 128.

TABLE 1
Number of differentially expressed genes in different comparisons

DE protein coding genes

| Condition | Cell type | Up | Down | Total |
|---|---|---|---|---|
| PD vs. HC_NR | PBMC | 7 | 11 | 18 |
| | CD4 | 1 | 10 | 11 |
| | CD8 | 4 | 5 | 9 |
| PD_R vs. PD_NR | PBMC | 18 | 72 | 90 |
| | CD4 | 168 | 136 | 304 |
| | CD8 | 284 | 49 | 333 |
| PD_R vs. HC_NR | PBMC | 19 | 46 | 65 |
| | CD4 | 81 | 91 | 172 |
| | CD8 | 192 | 35 | 227 |

PD, Parkinson's disease;
PD_R, PD responders to α-syn;
PD_NR, PD non-responders;
HCNR, Healthy control non-responders

TABLE 2
Characteristics of the subjects enrolled in the study

| | RNAseq Cohort | | | | | | Validation cohort | | | | | |
|---|---|---|---|---|---|---|---|---|---|---|---|---|
| | PD_R | | PD_NR | | HC_NR | | PD_R | | PD_NR | | HC_NR | |
| Total subjects | 15 | | 21 | | 20 | | 10 | | 10 | | 10 | |
| enrolled | | | | | | | | | | | | |
| Median age | 70, | (49-81) | 66, | (44-81) | 67, | (50-79) | 67 | (44-76) | 65 | (46-81) | 52 | (22-69) |
| (range), yr | | | | | | | | | | | | |
| Male, %(n) | 73.3% | (11) | 85.7% | (18) | 20% | (4) | 70% | (7) | 70% | (7) | 50% | (5) |
| Caucasian, % (n) | 88.8% | (32) | 80% | (16) | 20% | (4) | 90% | (9) | 100% | (10) | 50% | (5) |
| Median years | 3 | (0-12) | 6 | (0-16) | NA | | 7 | (0-12) | 9 | (0-20) | NA | |
| since diagnosis, | | | | | | | | | | | | |
| (range), yr | | | | | | | | | | | | |
| Median | 27 | (9-30) | 26 | (23-30) | NA | | 28 | (22-30) | 28 | (14-30) | NA | |
| MoCA^{a}(range) | | | | | | | | | | | | |
| Median UPDRS^{b} | 17 | (13-37) | 17 | (5-30) | NA | | 18 | (14-24) | 18 | (11-52) | NA | |
| (range) | | | | | | | | | | | | |

^{a}MoCA collected for n = 32 PD patients in the RNAseq cohort and n = 17 in the validation cohort
^{b}UPDRS collected for n = 31 PD patients in the RNAseq cohort and = 17 in the validation cohort.

TABLE 3
PBMC and CD4 memory

| | | PBMC | | | | | | CD4 memory | | | | | |
|---|---|---|---|---|---|---|---|---|---|---|---|---|---|
| | | PD vs HC | | PD_R vs PD_NR | | PD_R vs HC_NR | | PD vs HC | | PD_R vs PD_NR | | PD_R vs HC_NR | |
| | | log2 | adj p | log2 | adj p | log2 | adj p | log2 | adj p | log2 | adj p | log2 | adj p |
| Gene | gene type | fold change | value | fold change | value | fold change | value | fold change | value | fold change | value | fold change | value |
| SLC16A13 | protein_coding | | | | | | | | | −3.19 | 0.002 | | |
| POU5F2 | protein_coding | | | | | | | | | −3.19 | 0.0022 | | |
| TBC1D8 | protein_coding | | | | | | | | | −3.09 | 0.005 | | |
| C11orf65 | protein_coding | | | −1.82 | 0.04 | | | | | −3.07 | 0.002 | | |
| CX3CR1 | protein_coding | | | | | | | | | −3.05 | 0.016 | | |
| FCGBP | protein_coding | | | | | | | | | −3.02 | 0.0059 | | |
| TNFAIP8L2 | protein_coding | | | | | | | | | −3 | 7.1E−09 | −2.95 | 0.000000044 |
| CSF3R | protein_coding | | | | | | | | | −2.99 | 0.00029 | −2.48 | 0.012 |
| XPNPEP2 | protein_coding | | | −1.73 | 0.000000066 | | | | | −2.98 | 0.019 | | |
| PRAG1 | protein_coding | | | | | | | | | −2.84 | 0.00099 | −2.15 | 0.018 |
| CD8A | protein_coding | | | | | | | | | −2.8 | 0.016 | | |
| ARHGEF5 | protein_coding | | | | | | | | | −2.75 | 0.016 | | |
| SIGLEC7 | protein_coding | | | | | | | | | −2.75 | 0.0000012 | | |
| ZNF546 | protein_coding | | | | | | | | | −2.74 | 0.00067 | −2.53 | 0.0017 |
| AGAP6 | protein_coding | | | | | | | | | −2.67 | 0.002 | −2.18 | 0.031 |
| SRC | protein_coding | | | | | | | | | −2.67 | 0.027 | | |
| RAB20 | protein_coding | | | | | | | | | −2.6 | 0.0061 | | |
| AC051649.2 | protein_coding | | | | | | | | | −2.59 | 0.032 | | |
| DOK6 | protein_coding | | | | | | | | | −2.56 | 0.028 | | |
| CALHM2 | protein_coding | | | | | | | | | −2.53 | 0.0021 | | |
| WIPI1 | protein_coding | | | | | | | | | −2.53 | 0.00094 | −2.39 | 0.0038 |
| CORO1C | protein_coding | | | | | | | | | −2.52 | 0.034 | | |
| KLHL35 | protein_coding | | | | | | | | | −2.49 | 0.028 | | |
| HIST2H2AB | protein_coding | | | | | | | | | −2.46 | 0.0033 | | |
| E2F2 | protein_coding | | | | | | | | | −2.45 | 0.016 | | |
| LAT2 | protein_coding | | | | | | | | | −2.43 | 0.028 | −2.34 | 0.028 |
| MAMDC4 | protein_coding | | | | | | | | | −2.43 | 0.021 | | |
| TFEB | protein_coding | | | | | | | | | −2.3 | 0.016 | | |
| DUSP7 | protein_coding | | | | | | | | | −2.26 | 0.0076 | | |
| F2RL2 | protein_coding | | | | | | | | | −2.22 | 0.0024 | −1.8 | 0.022 |
| PTPN23 | protein_coding | | | | | | | | | −2.19 | 0.0083 | −2.14 | 0.019 |
| MMP25 | protein_coding | | | | | | | | | −2.17 | 0.018 | | |
| CISH | protein_coding | | | | | | | | | −2.08 | 0.025 | | |
| PSKH1 | protein_coding | | | | | | | | | −2.04 | 0.035 | | |
| TNFAIP2 | protein_coding | | | | | | | | | −2.04 | 0.0034 | | |
| HS6ST1 | protein_coding | | | | | | | | | −2.03 | 0.043 | | |
| PEX26 | protein_coding | | | | | | | | | −2.01 | 0.042 | | |
| INMT | protein_coding | | | | | | | | | −1.95 | 3.6E−47 | | |
| AP2A1 | protein_coding | | | | | | | | | −1.93 | 0.0031 | −1.9 | 0.0092 |
| ZNF175 | protein_coding | | | | | | | | | −1.93 | 3.9E−54 | −1.38 | 8.2E−46 |
| RAB3D | protein_coding | | | | | | | | | −1.91 | 0.021 | | |
| CDC42EP2 | protein_coding | | | | | | | | | −1.89 | 0.00067 | | |
| FGR | protein_coding | | | | | | | | | −1.84 | 0.049 | | |
| FAM131B | protein_coding | | | | | | | | | −1.79 | 9.1E−49 | −0.98 | 4.9E−39 |
| PRKN | protein_coding | | | | | | | | | −1.79 | 0.029 | | |
| ADGRG1 | protein_coding | | | | | | | | | −1.77 | 0.039 | | |
| ACAN | protein_coding | | | | | | | | | −1.75 | 0.00024 | −0.91 | 0.0031 |
| QRICH2 | protein_coding | | | | | | | | | −1.73 | 0.00031 | | |
| SEPT5 | protein_coding | | | | | | | | | −1.69 | 9.1E−33 | −1.78 | 4.8E−30 |
| CCNB2 | protein_coding | | | | | | | | | −1.68 | 3.8E−09 | −1.94 | 7.5E−09 |
| ABCC3 | protein_coding | | | | | | | | | −1.61 | 8.6E−40 | −0.72 | 1.9E−26 |
| ZNF418 | protein_coding | | | | | | | | | −1.58 | 0.0000027 | −0.82 | 0.000088 |
| C5orf34 | protein_coding | | | | | | | | | −1.55 | 0.027 | | |
| CASP2 | protein_coding | | | | | | | | | −1.51 | 0.0055 | −1.79 | 0.0019 |
| FBLN2 | protein_coding | | | | | | | | | −1.5 | 2.1E−32 | | |
| MIER2 | protein_coding | | | | | | | | | −1.49 | 0.028 | | |
| MICAL3 | protein_coding | | | | | | | | | −1.48 | 0.013 | −2.29 | 0.0001 |
| DHCR24 | protein_coding | | | | | | | | | −1.48 | 0.000000093 | −1.46 | 0.00000055 |
| CD36 | protein_coding | | | | | | | | | −1.47 | 0.0082 | −2.14 | 0.00082 |
| PYGO2 | protein_coding | | | | | | | | | −1.44 | 0.041 | | |
| GINS1 | protein_coding | | | | | | | | | −1.43 | 1.7E−47 | −0.78 | 2.6E−37 |
| RAB6B | protein_coding | | | | | | | | | −1.42 | 2.7E−41 | −1.31 | 1E−29 |
| TMEM201 | protein_coding | | | | | | | | | −1.4 | 0.019 | −1.31 | 0.039 |
| IGFBP6 | protein_coding | | | | | | | | | −1.38 | 0.00015 | −1.28 | 0.000068 |
| TPD52 | protein_coding | | | | | | | | | −1.38 | 0.0042 | | |
| PTGDR2 | protein_coding | | | | | | | −1.33 | 0.00024 | −1.37 | 0.0077 | −2.72 | 0.000012 |
| KCNN3 | protein_coding | | | | | | | | | −1.37 | 0.019 | | |
| CDA | protein_coding | | | | | | | | | −1.36 | 0.00085 | −1.51 | 0.014 |
| ITGB4 | protein_coding | | | | | | | | | −1.32 | 0.023 | | |
| WBP2NL | protein_coding | | | | | | | | | −1.27 | 0.0088 | −1.63 | 0.017 |
| TTC30A | protein_coding | | | | | | | | | −1.22 | 7.7E−38 | −1.38 | 1.3E−36 |
| ZNF45 | protein_coding | | | | | | | | | −1.2 | 0.007 | | |
| CEBPE | protein_coding | | | | | | | | | −1.19 | 1.5E−33 | −1.01 | 2.5E−31 |
| MRGPRD | protein_coding | | | | | | | | | −1.18 | 1.1E−17 | | |
| SPINK4 | protein_coding | | | | | | | | | −1.17 | 0.032 | | |
| ABO | protein_coding | | | | | | | | | −1.16 | 0.025 | −1.17 | 0.045 |
| SELPLG | protein_coding | | | | | | | | | −1.15 | 0.0095 | | |
| C14orf79 | protein_coding | | | | | | | | | −1.12 | 1.7E−50 | −1.16 | 4.3E−45 |
| COL16A1 | protein_coding | | | | | | | | | −1.1 | 4.1E−43 | −1.13 | 7.5E−34 |
| PDZD7 | protein_coding | | | | | | | | | −1.1 | 0.048 | | |
| TOM1L1 | protein_coding | | | | | | | | | −1.1 | 5.4E−32 | −0.65 | 9.8E−23 |
| HHLA2 | protein_coding | | | | | | | | | −1.09 | 1.6E−17 | | |
| MFSD8 | protein_coding | | | | | | | | | −1.09 | 0.036 | | |
| KCNH4 | protein_coding | | | | | | | | | −1.08 | 5.3E−46 | −1.95 | 5.2E−43 |
| DCDC2B | protein_coding | | | | | | | | | −1.06 | 3.6E−47 | −1.6 | 1.7E−41 |
| ST6GALNAC6 | protein_coding | | | | | | | | | −1.04 | 0.021 | | |
| TPD52L1 | protein_coding | | | | | | | | | −1.04 | 3.5E−36 | −0.82 | 2.9E−24 |
| ARRDC5 | protein_coding | | | | | | | | | −1.02 | 0.048 | −1.78 | 0.0022 |
| RAB1B | protein_coding | | | | | | | | | −1.02 | 0.008 | | |
| ACAD9 | protein_coding | | | | | | | | | −1.01 | 0.0045 | | |
| RNF152 | protein_coding | | | | | | | | | −0.98 | 4.8E−50 | −1.13 | 3E−38 |
| ZFHX2 | protein_coding | | | | | | | | | −0.97 | 8.4E−15 | −0.6 | 1.1E−12 |
| CPB2 | protein_coding | | | | | | | | | −0.92 | 1.4E−26 | −0.72 | 5.3E−21 |
| CD300LB | protein_coding | | | | | | | | | −0.92 | 2.3E−17 | −0.87 | 7.8E−14 |
| ZNF620 | protein_coding | | | −1.65 | 0.00052 | −1.34 | 0.015 | | | −0.91 | 0.0038 | | |
| FAM227A | protein_coding | | | | | | | | | −0.91 | 8.4E−24 | | |
| FFAR3 | protein_coding | | | | | | | | | −0.88 | 1E−17 | −0.59 | 1.8E−18 |
| SGCA | protein_coding | | | | | | | | | −0.88 | 4.9E−23 | | |
| AL022238.4 | protein_coding | | | | | | | | | −0.85 | 9.5E−13 | | |
| PBXIP1 | protein_coding | | | | | | | | | −0.85 | 0.0025 | | |
| LRFN2 | protein_coding | | | | | | | | | −0.83 | 6E−19 | | |
| SLC7A8 | protein_coding | | | | | | | | | −0.82 | 0.01 | −1.94 | 0.0029 |
| TAP1 | protein_coding | | | | | | | | | −0.81 | 0.023 | | |
| IMPA2 | protein_coding | | | | | | | | | −0.8 | 0.045 | | |
| RUFY4 | protein_coding | | | | | | | | | −0.79 | 9.2E−15 | | |
| CHRNA10 | protein_coding | | | | | | | | | −0.78 | 0.0078 | −0.8 | 0.03 |
| CA2 | protein_coding | | | | | | | | | −0.75 | 1.6E−41 | −1.55 | 8.5E−32 |
| EXOC3L2 | protein_coding | | | | | | | | | −0.74 | 4.2E−10 | | |
| RET | protein_coding | | | | | | | | | −0.74 | 2E−12 | | |
| IL22 | protein_coding | | | | | | | | | −0.73 | 1.1E−17 | −0.7 | 6.3E−18 |
| ST3GAL6 | protein_coding | | | | | | | | | −0.73 | 8.4E−12 | | |
| ARHGEF28 | protein_coding | | | | | | | | | −0.72 | 4.2E−10 | | |
| SLC15A2 | protein_coding | | | | | | | | | −0.71 | 4.9E−16 | −0.63 | 1.6E−10 |
| GPR153 | protein_coding | | | | | | | | | −0.69 | 2E−11 | | |
| TTC26 | protein_coding | | | | | | | | | −0.69 | 2.9E−38 | −2.38 | 2.1E−42 |
| WEE2 | protein_coding | | | | | | | | | −0.69 | 8.2E−10 | | |
| MED15 | protein_coding | | | | | | | | | −0.68 | 0.013 | | |
| KIRREL2 | protein_coding | | | | | | | | | −0.68 | 2.7E−09 | | |
| MS4A14 | protein_coding | | | | | | | | | −0.68 | 8.9E−11 | | |
| CCDC194 | protein_coding | | | | | | | | | −0.67 | 2.9E−10 | | |
| ADGRE5 | protein_coding | | | | | | | | | −0.67 | 0.0027 | −0.76 | 0.0023 |
| LRRK2 | protein_coding | | | | | | | | | −0.67 | 5.3E−19 | −1.46 | 4.5E−18 |
| SLC30A8 | protein_coding | | | | | | | | | −0.66 | 0.000000064 | | |
| LYPD4 | protein_coding | | | | | | | | | −0.65 | 0.000000064 | | |
| MPO | protein_coding | | | | | | | | | −0.65 | 2.1E−13 | | |
| OLFML2B | protein_coding | | | | | | | | | −0.65 | 2.2E−09 | | |
| GML | protein_coding | | | | | | | | | −0.64 | 2.4E−09 | | |
| FGFBP1 | protein_coding | | | | | | | | | −0.64 | 0.0013 | | |
| IGDCC4 | protein_coding | | | | | | | | | −0.64 | 1.5E−11 | | |
| PPP1R26 | protein_coding | | | | | | | | | −0.62 | 0.000000015 | | |
| MGAM2 | protein_coding | | | | | | | | | −0.62 | 6.8E−09 | | |
| HMGCS2 | protein_coding | | | | | | | | | −0.61 | 0.000011 | | |
| GPR42 | protein_coding | | | | | | | | | −0.61 | 0.0000031 | | |
| ZNF670 | protein_coding | | | | | | | | | −0.61 | 0.022 | | |
| KRBOX1 | protein_coding | | | | | | | | | −0.6 | 0.00002 | | |
| METTL21C | protein_coding | | | | | | | | | −0.59 | 0.0000066 | | |
| LSMEM1 | protein_coding | | | | | | | | | 1.63 | 0.000000069 | 1.37 | 0.0000068 |
| RASD1 | protein_coding | | | | | | | | | 0.63 | 0.0018 | | |
| LEAP2 | protein_coding | | | | | | | | | 3.33 | 0.000000046 | 2.23 | 0.0012 |
| AIG1 | protein_coding | | | | | | | | | 1.7 | 0.000031 | 1.25 | 0.0069 |
| IL6R | protein_coding | | | | | | | | | 1.76 | 0.000037 | | |
| ASAH2 | protein_coding | | | | | | | | | 0.71 | 1.1E−11 | | |
| GDF11 | protein_coding | | | | | | | | | 2.19 | 0.000016 | 1.49 | 0.022 |
| B3GNT8 | protein_coding | | | | | | | | | 0.73 | 0.0039 | | |
| STRC | protein_coding | | | | | | | | | 0.75 | 1.1E−09 | 0.72 | 0.0032 |
| ABCD2 | protein_coding | | | | | | | | | 1.87 | 0.00017 | 1.56 | 0.0012 |
| CELSR2 | protein_coding | | | | | | | | | 1.16 | 0.00099 | 1.67 | 0.000085 |
| SFSWAP | protein_coding | | | | | | | | | 0.82 | 0.0073 | | |
| ELOA | protein_coding | | | | | | | | | 0.85 | 0.035 | | |
| NKAPL | protein_coding | | | | | | | | | 0.85 | 1.3E−09 | 0.82 | 0.00067 |
| TCAIM | protein_coding | | | | | | | | | 0.85 | 0.038 | | |
| TOMM5 | protein_coding | | | | | | | | | 0.85 | 0.027 | 0.9 | 0.047 |
| PTTG1 | protein_coding | | | | | | | | | 0.87 | 0.03 | | |
| E2F1 | protein_coding | | | | | | | | | 0.88 | 0.023 | | |
| ZNF74 | protein_coding | | | | | | | | | 0.9 | 0.0064 | 1.62 | 0.003 |
| RNASEL | protein_coding | | | | | | | | | 0.92 | 0.05 | | |
| PPARGC1B | protein_coding | | | | | | | | | 0.93 | 0.021 | 0.85 | 0.026 |
| APOL1 | protein_coding | | | | | | | | | 1.67 | 0.00011 | 1.95 | 0.00013 |
| PXMP2 | protein_coding | | | | | | | | | 1.97 | 0.0011 | | |
| ZNF182 | protein_coding | | | | | | | | | 0.97 | 0.013 | | |
| ZFP69B | protein_coding | | | | | | | | | 1 | 0.0082 | 1.37 | 0.0045 |
| FUT11 | protein_coding | | | | | | | | | 1.01 | 0.026 | | |
| EEPD1 | protein_coding | | | | | | | | | 1.03 | 0.024 | | |
| INVS | protein_coding | | | | | | | | | 1.04 | 0.027 | 1.32 | 0.047 |
| P3H4 | protein_coding | | | −1.43 | 0.0046 | −1.54 | 0.019 | | | 1.05 | 0.00088 | 0.68 | 0.0096 |
| LARGE2 | protein_coding | | | | | | | | | 1.06 | 0.00063 | 0.85 | 0.019 |
| LAMP3 | protein_coding | | | | | | | | | 1.71 | 0.0015 | 0.96 | 0.031 |
| FAM213A | protein_coding | | | | | | | | | 1.19 | 0.0022 | 0.97 | 0.031 |
| EPHX1 | protein_coding | | | | | | | | | 1.07 | 0.02 | | |
| HIST1H4H | protein_coding | | | | | | | | | 1.07 | 0.027 | 1.19 | 0.05 |
| ECE2 | protein_coding | | | | | | | | | 1.23 | 0.0022 | 0.67 | 0.036 |
| LLGL1 | protein_coding | | | | | | | | | 1.09 | 0.035 | | |
| BCL2 | protein_coding | | | | | | | | | 1.42 | 0.0032 | | |
| ANKRD35 | protein_coding | | | | | | | | | 1.1 | 0.0027 | 1.23 | 0.0022 |
| HOOK2 | protein_coding | | | | | | | | | 1.1 | 0.048 | | |
| SAMD12 | protein_coding | | | | | | | | | 1.1 | 0.019 | 1.24 | 0.017 |
| TRPM2 | protein_coding | | | | | | | | | 1.1 | 0.0035 | 1.13 | 0.0033 |
| HYLS1 | protein_coding | | | | | | | | | 1.11 | 0.039 | 1.63 | 0.015 |
| GYPE | protein_coding | | | | | | | | | 0.61 | 0.0052 | | |
| CD180 | protein_coding | | | | | | | | | 0.65 | 0.0053 | | |
| CASK | protein_coding | | | | | | | | | 1.76 | 0.0074 | 2.3 | 0.0024 |
| TWISTNB | protein_coding | | | | | | | | | 1.14 | 0.0072 | | |
| CLYBL | protein_coding | | | | | | | | | 1.64 | 0.008 | | |
| MTUS1 | protein_coding | | | | | | | | | 1.48 | 0.0081 | 1.2 | 0.031 |
| ROPN1L | protein_coding | | | | | | | | | 1.16 | 0.000053 | 0.65 | 0.0022 |
| DCST1 | protein_coding | | | | | | | | | 1.65 | 0.0085 | 1.95 | 0.023 |
| ISM1 | protein_coding | | | | | | | | | 1.49 | 0.00071 | | |
| ZXDB | protein_coding | | | | | | | | | 1.18 | 0.05 | | |
| CSF2RB | protein_coding | | | | | | | | | 1.07 | 0.01 | | |
| MPP5 | protein_coding | | | | | | | | | 1.19 | 0.047 | | |
| ALCAM | protein_coding | | | | | | | | | 1.8 | 0.011 | | |
| ACOT4 | protein_coding | | | | | | | | | 1.21 | 0.0036 | | |
| TCF19 | protein_coding | | | | | | | | | 1.21 | 0.043 | | |
| DOK4 | protein_coding | | | | | −0.76 | 0.039 | | | 1.22 | 0.0046 | | |
| PET100 | protein_coding | | | | | | | | | 1.22 | 0.047 | | |
| ZC4H2 | protein_coding | | | | | | | | | 1.22 | 0.008 | | |
| LMO7 | protein_coding | | | | | | | | | 1.41 | 0.014 | | |
| LIPT1 | protein_coding | | | | | | | | | 1.23 | 0.027 | | |
| NAP1L2 | protein_coding | | | | | | | | | 1.23 | 0.046 | | |
| TMEM97 | protein_coding | | | | | | | | | 1.21 | 0.015 | 1.36 | 0.033 |
| ELP1 | protein_coding | | | | | | | | | 1.24 | 0.029 | | |
| AAMDC | protein_coding | | | | | | | | | 1.26 | 0.026 | | |
| SVIL | protein_coding | | | | | | | | | 1.26 | 0.0086 | | |
| KNSTRN | protein_coding | | | | | | | | | 1.27 | 0.0027 | 1.26 | 0.006 |
| NSUN6 | protein_coding | | | | | | | | | 1.27 | 0.047 | | |
| SARNP | protein_coding | | | | | | | | | 1.27 | 0.028 | 2.53 | 0.000046 |
| POLA1 | protein_coding | | | | | | | | | 1.28 | 0.015 | | |
| RNMT | protein_coding | | | | | | | | | 1.28 | 0.0082 | | |
| CRB3 | protein_coding | | | | | | | | | 1.43 | 0.015 | 1.62 | 0.009 |
| PDIA5 | protein_coding | | | | | | | | | 1.29 | 0.000031 | 1.23 | 0.00013 |
| ALS2 | protein_coding | | | | | | | | | 1.31 | 0.0092 | | |
| LYPD8 | protein_coding | | | | | | | | | 0.64 | 0.004 | 0.68 | 0.000000087 |
| MYEF2 | protein_coding | | | | | | | | | 1.32 | 0.012 | 1.58 | 0.019 |
| P3H3 | protein_coding | | | | | | | | | 1.32 | 0.0006 | 2 | 0.0003 |
| PACRGL | protein_coding | | | | | | | | | 1.33 | 0.0014 | | |
| H6PD | protein_coding | | | | | | | | | 1.35 | 0.034 | 1.57 | 0.025 |
| PIGK | protein_coding | | | | | | | | | 1.36 | 0.016 | | |
| DBNDD1 | protein_coding | | | | | | | | | 1.36 | 0.007 | | |
| CKS1B | protein_coding | | | | | | | | | 1.37 | 0.036 | | |
| TTC13 | protein_coding | | | | | | | | | 0.95 | 0.02 | 1.67 | 0.0018 |
| PEX11G | protein_coding | | | | | | | | | 1.13 | 0.02 | | |
| BIRC2 | protein_coding | | | | | | | | | 1.38 | 0.0084 | | |
| NRIP3 | protein_coding | | | | | | | | | 1.38 | 0.012 | | |
| COPG2 | protein_coding | | | | | | | | | 1.39 | 0.00092 | | |
| PPP2R2A | protein_coding | | | | | | | | | 1.39 | 0.015 | | |
| HMBOX1 | protein_coding | | | | | | | | | 1.4 | 0.041 | | |
| MRPL1 | protein_coding | | | | | | | | | 1.4 | 0.012 | | |
| NEK11 | protein_coding | | | | | | | | | 1.4 | 0.013 | | |
| SLC7A10 | protein_coding | | | | | | | | | 0.78 | 0.021 | | |
| NDUFC1 | protein_coding | | | | | | | | | 1.13 | 0.021 | | |
| CHMP5 | protein_coding | | | | | | | | | 1.42 | 0.016 | | |
| ZNF805 | protein_coding | | | | | | | | | 1.42 | 0.0049 | 1.53 | 0.015 |
| CREB3L4 | protein_coding | | | | | | | | | 1.16 | 0.021 | | |
| IFT22 | protein_coding | | | | | | | | | 1.43 | 0.038 | | |
| ADAM22 | protein_coding | | | | | | | | | 1.29 | 0.022 | | |
| TAS1R3 | protein_coding | | | | | | | | | 0.81 | 0.026 | 0.81 | 0.0016 |
| PRKARIB | protein_coding | | | | | | | | | 1.44 | 0.024 | | |
| ZNF532 | protein_coding | | | | | | | | | 1.46 | 0.000066 | 0.97 | 0.0081 |
| C17orf51 | protein_coding | | | | | | | | | 1.47 | 0.023 | 1.67 | 0.037 |
| HIST2H2AC | protein_coding | | | | | | | | | 1.47 | 0.016 | | |
| SLC35G1 | protein_coding | | | | | | | | | 0.68 | 0.028 | | |
| DISC1 | protein_coding | | | | | | | | | 1.49 | 0.013 | | |
| MGP | protein_coding | | | | | | | | | 1.31 | 0.0098 | | |
| PTPDC1 | protein_coding | | | | | | | | | 1.5 | 0.011 | | |
| LRRC29 | protein_coding | | | | | | | | | 1.52 | 0.0019 | | |
| MTRF1L | protein_coding | | | | | | | | | 1.52 | 0.043 | | |
| ZBTB41 | protein_coding | | | | | | | | | 1.53 | 0.0031 | 1.69 | 0.024 |
| FARP1 | protein_coding | | | | | | | | | 1.57 | 0.012 | | |
| TTC12 | protein_coding | | | | | | | | | 1.57 | 0.018 | | |
| AC003002.1 | protein_coding | | | | | | | | | 1.58 | 0.0052 | | |
| PAXIP1 | protein_coding | | | | | | | | | 1.58 | 0.00038 | 1.07 | 0.031 |
| AP2A2 | protein_coding | | | | | | | | | 1.59 | 0.05 | | |
| BCKDHB | protein_coding | | | | | | | | | 1.59 | 0.023 | | |
| DYNC2H1 | protein_coding | | | | | | | | | 1.59 | 0.0052 | | |
| NLRP2 | protein_coding | | | | | | | | | 1.59 | 0.047 | | |
| NUDT8 | protein_coding | | | | | | | | | 1.59 | 0.0051 | | |
| CCDC138 | protein_coding | | | | | | | | | 1.6 | 0.025 | | |
| ZNF248 | protein_coding | | | | | | | | | 1.6 | 0.0063 | | |
| SERPINH1 | protein_coding | | | | | | | | | 1.61 | 0.00013 | 0.97 | 0.014 |
| TMEM250 | protein_coding | | | | | | | | | 0.73 | 0.028 | | |
| ABAT | protein_coding | | | | | | | | | 1.63 | 0.011 | | |
| CPNE2 | protein_coding | | | | | | | | | 1.63 | 0.0095 | | |
| SLC25A19 | protein_coding | | | | | | | | | 1.07 | 0.028 | | |
| LRRC42 | protein_coding | | | | | | | | | 1.64 | 0.0033 | | |
| ZCCHC4 | protein_coding | | | | | | | | | 1.64 | 0.021 | | |
| GCNT2 | protein_coding | | | | | | | | | 1.24 | 0.028 | | |
| GCFC2 | protein_coding | | | | | | | | | 1.65 | 0.01 | | |
| ZNF594 | protein_coding | | | | | | | | | 1.66 | 0.000063 | 0.91 | 0.014 |
| CYP2S1 | protein_coding | | | | | | | | | 1.09 | 0.029 | 1.88 | 0.000065 |
| ZFYVE26 | protein_coding | | | | | | | | | 1.68 | 0.0044 | | |
| BIRC5 | protein_coding | | | | | | | | | 1.69 | 0.0028 | | |
| SPAG5 | protein_coding | | | | | | | | | 1.69 | 0.024 | | |
| ZNF17 | protein_coding | | | | | | | | | 1.69 | 0.02 | | |
| CERS6 | protein_coding | | | | | | | | | 1.11 | 0.033 | 1.33 | 0.031 |
| XXYLT1 | protein_coding | | | | | | | | | 1.7 | 0.000046 | 1.23 | 0.0032 |
| MAN1C1 | protein_coding | | | | | | | | | 1.12 | 0.033 | | |
| MYL6B | protein_coding | | | | | | | | | 1.73 | 0.0055 | | |
| RSPH3 | protein_coding | | | | | | | | | 1.73 | 0.004 | | |
| KCNQ1 | protein_coding | | | | | | | | | 1.44 | 0.034 | | |
| CYP4V2 | protein_coding | | | | | | | | | 1.18 | 0.037 | | |
| DENND1A | protein_coding | | | | | | | | | 1.38 | 0.039 | | |
| ACO1 | protein_coding | | | | | | | | | 1.78 | 0.012 | 1.94 | 0.0077 |
| FAM171A1 | protein_coding | | | | | | | | | 1.44 | 0.044 | | |
| MAFG | protein_coding | | | | | | | | | 1.8 | 0.0091 | 3.2 | 0.000072 |
| MPHOSPH9 | protein_coding | | | | | | | | | 1.8 | 0.00084 | 1.68 | 0.017 |
| FAM19A2 | protein_coding | | | | | | | | | 1.83 | 0.0022 | 1.73 | 0.016 |
| ADCK5 | protein_coding | | | | | | | | | 1.75 | 0.044 | | |
| AC069544.2 | protein_coding | | | | | | | | | 1.91 | 0.019 | | |
| KLHL32 | protein_coding | | | | | | | | | 1.95 | 0.0009 | 2.26 | 0.0003 |
| ZCCHC18 | protein_coding | | | | | | | | | 1.96 | 0.00028 | 1.22 | 0.012 |
| DNAJC25 | protein_coding | | | | | | | | | 1.38 | 0.048 | | |
| TCFL5 | protein_coding | | | | | | | | | 2.04 | 0.011 | | |
| PLEKHA7 | protein_coding | | | | | | | | | 2.05 | 0.00078 | 1.85 | 0.0048 |
| HIST2H2BF | protein_coding | | | | | | | | | 2.06 | 0.013 | | |
| CNKSR2 | protein_coding | | | | | | | | | 2.09 | 0.002 | | |
| CENPE | protein_coding | | | | | | | | | 2.14 | 0.00039 | 1.94 | 0.0015 |
| ZNF284 | protein_coding | | | | | | | | | 2.14 | 0.0019 | 1.83 | 0.019 |
| ADCK1 | protein_coding | | | | | | | | | 0.94 | 0.022 | | |
| UBAP1L | protein_coding | | | | | | | | | 2.19 | 0.0014 | 2.51 | 0.00057 |
| B3GALNT2 | protein_coding | | | | | | | | | 2.2 | 0.0014 | | |
| NRIP2 | protein_coding | | | | | | | | | 2.21 | 0.004 | | |
| MECR | protein_coding | | | | | | | | | 2.22 | 0.00022 | | |
| BAIAP2 | protein_coding | | | | | | | | | 2.7 | 0.00000074 | 2.04 | 0.013 |
| MEGF6 | protein_coding | | | | | | | | | 1.18 | 0.038 | | |
| AQP9 | protein_coding | | | | | | | | | | | 0.59 | 0.0005 |
| SCARA3 | protein_coding | | | | | | | | | | | 0.9 | 0.0007 |
| SLC35F3 | protein_coding | | | | | | | | | | | 0.73 | 0.0064 |
| TNFRSF11A | protein_coding | | | | | | | | | | | 1.75 | 0.021 |
| TOMM20L | protein_coding | | | | | | | | | | | 0.64 | 0.024 |
| GALNT1 | protein_coding | | | | | | | | | | | 1.87 | 0.028 |
| HMOX1 | protein_coding | | | | | | | | | | | | |
| MCOLN3 | protein_coding | | | | | | | | | | | 0.91 | 0.036 |
| RNF222 | protein_coding | | | | | | | | | | | | |
| CYP2F1 | protein_coding | 1.59 | 0.041 | | | 1.58 | 0.025 | | | | | | |
| PDPR | protein_coding | | | | | 1.54 | 0.042 | | | | | | |
| P2RY6 | protein_coding | | | −1.65 | 0.013 | −1.22 | 0.016 | | | | | | |
| EIF1AY | protein_coding | 1.51 | 0.011 | | | 1.48 | 0.023 | | | | | | |
| OGFOD2 | protein_coding | | | | | 1.43 | 0.028 | | | | | | |
| METTL16 | protein_coding | | | | | 1.39 | 0.009 | | | | | | |
| CCR5 | protein_coding | | | −0.95 | 0.031 | −1.21 | 0.012 | | | | | | |
| NOP2 | protein_coding | | | | | 1.23 | 0.046 | | | | | | |
| PNMA5 | protein_coding | 1.3 | 0.03 | | | 1.19 | 0.027 | | | | | | |
| CDK17 | protein_coding | | | 1.03 | 0.0089 | 1.15 | 0.032 | | | | | | |
| ETFBKMT | protein_coding | | | | | 1.13 | 0.029 | | | | | | |
| ZC3H18 | protein_coding | | | 0.9 | 0.028 | 1.11 | 0.033 | | | | | | |
| FUT2 | protein_coding | | | | | −1.15 | 0.02 | | | | | | |
| SECISBP2L | protein_coding | | | | | 1.03 | 0.038 | | | | | | |
| CERK | protein_coding | | | | | 0.87 | 0.02 | | | | | | |
| USP2 | protein_coding | | | | | 0.79 | 0.02 | | | | | | |
| LPIN3 | protein_coding | | | | | 0.76 | 0.000000023 | | | | | | |
| GCM1 | protein_coding | | | | | 0.65 | 0.042 | | | | | | |
| PCBP2 | protein_coding | | | | | 0.64 | 0.039 | | | | | | |
| ARNTL2 | protein_coding | | | | | −0.58 | 0.0019 | | | | | | |
| MFSD2B | protein_coding | −1.23 | 0.0073 | | | −1.04 | 0.037 | | | | | | |
| NRP2 | protein_coding | −1 | 0.00025 | | | −0.97 | 0.00062 | | | | | | |
| ACE | protein_coding | | | | | −0.97 | 0.038 | | | | | | |
| CTRC | protein_coding | | | | | −0.79 | 0.002 | | | | | | |
| SLC22A16 | protein_coding | | | −1.5 | 0.0004 | −0.88 | 0.043 | | | | | | |
| PTK6 | protein_coding | | | | | −0.65 | 0.047 | | | | | | |
| GSC | protein_coding | | | −0.87 | 0.0038 | −0.66 | 0.048 | | | | | | |
| ZNF835 | protein_coding | | | | | −0.66 | 0.0029 | | | | | | |
| PRSS27 | protein_coding | | | | | −0.69 | 0.017 | | | | | | |
| TMTC1 | protein_coding | −0.8 | 0.0012 | | | −0.82 | 0.0022 | | | | | | |
| COL4A2 | protein_coding | | | | | −0.63 | 0.00014 | | | | | | |
| DCHS1 | protein_coding | −0.7 | 0.027 | | | −0.82 | 0.015 | | | | | | |
| CCR1 | protein_coding | | | | | | | | | | | | |
| SDCBP2 | protein_coding | −1 | 0.039 | | | −0.84 | 0.03 | | | | | | |
| ALG1L2 | protein_coding | | | | | −0.85 | 7.1E−36 | | | | | | |
| HFE | protein_coding | | | −1.21 | 0.000016 | −0.64 | 0.035 | | | | | | |
| GLS2 | protein_coding | −0.9 | 0.0035 | | | −0.9 | 0.0021 | | | | | | |
| DNM3 | protein_coding | | | | | −0.95 | 0.05 | | | | | | |
| FFAR4 | protein_coding | | | | | −0.64 | 0.000026 | | | | | | |
| FFAR1 | protein_coding | | | | | −0.61 | 0.0009 | | | | | | |
| HIST1H2AL | protein_coding | | | −1.07 | 0.023 | −1.01 | 0.015 | | | | | | |
| FSD1 | protein_coding | | | −1.38 | 0.0000004 | −1.03 | 0.000072 | | | | | | |
| PLPP7 | protein_coding | | | | | −0.59 | 0.000044 | | | | | | |
| EPHX3 | protein_coding | −1.05 | 0.00035 | | | −1.05 | 6.6E−27 | | | | | | |
| MRPL15 | protein_coding | | | | | −1.1 | 0.027 | | | | | | |
| AMOTL1 | protein_coding | | | −1.43 | 0.013 | −1.12 | 0.0081 | | | | | | |
| RPP25 | protein_coding | | | −1.31 | 0.00098 | −1.15 | 0.0057 | | | | | | |
| LPAR1 | protein_coding | | | | | 1.04 | 0.018 | | | | | | |
| TIGD6 | protein_coding | | | −1.12 | 0.029 | −1.2 | 0.00046 | | | | | | |
| CRIM1 | protein_coding | | | | | 1.38 | 0.048 | | | | | | |
| PEX3 | protein_coding | | | | | 1.52 | 0.027 | | | | | | |
| AL031708.1 | protein_coding | | | | | −1.24 | 0.042 | | | | | | |
| SPC24 | protein_coding | | | −1.44 | 0.0038 | −1.26 | 0.033 | | | | | | |
| PRPF40B | protein_coding | | | −1.58 | 0.0034 | −1.32 | 0.0098 | | | | | | |
| ADAL | protein_coding | | | | | −1.36 | 0.049 | | | | | | |
| PPAT | protein_coding | | | | | −1.48 | 0.041 | | | | | | |
| NR1H3 | protein_coding | | | −1.44 | 0.00077 | −1.55 | 0.011 | | | | | | |
| ZNF2 | protein_coding | | | −1.94 | 0.0015 | −1.67 | 0.02 | | | | | | |
| CAD | protein_coding | | | −1.67 | 0.0021 | −1.78 | 0.022 | | | | | | |
| BTBD3 | protein_coding | | | −1.72 | 0.000014 | −1.79 | 0.00028 | | | | | | |
| ZNF75D | protein_coding | | | −1.82 | 0.00039 | −1.87 | 0.0064 | | | | | | |
| ZSCAN9 | protein_coding | | | −1.3 | 0.022 | −1.88 | 0.0035 | | | | | | |
| GCNT7 | protein_coding | | | 2.03 | 0.0031 | | | | | | | | |
| ACTA1 | protein_coding | | | 2 | 0.0035 | | | | | | | | |
| POPDC2 | protein_coding | | | −2.53 | 0.000045 | | | | | | | | |
| NAPSA | protein_coding | | | −1.61 | 0.0012 | | | | | | | | |
| SNX32 | protein_coding | | | 1.67 | 0.013 | | | | | | | | |
| CTDSPL | protein_coding | | | 1.25 | 0.039 | | | | | | | | |
| SRGAP2 | protein_coding | | | 1.25 | 0.0036 | | | | | | | | |
| SPOCD1 | protein_coding | | | 1.21 | 0.03 | | | | | | | | |
| CLPX | protein_coding | | | 1.14 | 0.0052 | | | | | | | | |
| ZNF700 | protein_coding | | | 1.02 | 0.026 | | | | | | | | |
| GPR171 | protein_coding | | | −1.93 | 0.0025 | | | | | | | | |
| POLR3E | protein_coding | | | 0.81 | 0.013 | | | | | | | | |
| NUDT4 | protein_coding | | | 0.79 | 0.027 | | | | | | | | |
| DKK3 | protein_coding | | | −1.49 | 0.011 | | | | | | | | |
| TPR | protein_coding | | | 0.73 | 0.047 | | | | | | | | |
| MATR3 | protein_coding | | | 0.71 | 0.039 | | | | | | | | |
| ZNF385C | protein_coding | | | −0.64 | 0.00044 | | | | | | | | |
| HIGD1A | protein_coding | | | −0.66 | 0.043 | | | | | | | | |
| KDF1 | protein_coding | | | −0.68 | 0.038 | | | | | | | | |
| AATK | protein_coding | | | −1.76 | 0.0056 | | | | | | | | |
| ZC3H12C | protein_coding | | | −0.69 | 0.042 | | | | | | | | |
| SLC45A4 | protein_coding | | | −1.58 | 0.0018 | | | | | | | | |
| CLIP3 | protein_coding | | | −0.86 | 0.0021 | | | | | | | | |
| FAM173B | protein_coding | | | −1.55 | 0.049 | | | | | | | | |
| CAMSAP2 | protein_coding | | | −0.91 | 0.0047 | | | | | | | | |
| TGFB1I1 | protein_coding | | | −0.92 | 0.0032 | | | | | | | | |
| LINC01125 | protein_coding | | | −1.55 | 0.018 | | | | | | | | |
| KLF1 | protein_coding | | | −0.95 | 3.7E−58 | | | | | | | | |
| STK32B | protein_coding | | | −0.96 | 4.6E−29 | | | | | | | | |
| LTK | protein_coding | | | | | | | | | | | | |
| MADCAM1 | protein_coding | | | −1.29 | 0.00049 | | | | | | | | |
| ZC3H12B | protein_coding | | | −1.06 | 0.019 | | | | | | | | |
| C1QC | protein_coding | | | −1.35 | 0.031 | | | | | | | | |
| WRNIP1 | protein_coding | | | −1.09 | 0.0014 | | | | | | | | |
| CCDC34 | protein_coding | | | −1.11 | 0.00000027 | | | | | | | | |
| HAS1 | protein_coding | | | −1.23 | 0.015 | | | | | | | | |
| SH3BGRL2 | protein_coding | | | −1.21 | 0.0049 | | | | | | | | |
| ORMDL2 | protein_coding | | | −1.17 | 0.019 | | | | | | | | |
| C17orf80 | protein_coding | | | −0.97 | 0.019 | | | | | | | | |
| PLCD3 | protein_coding | | | −1.31 | 0.049 | | | | | | | | |
| EMID1 | protein_coding | | | −1.07 | 3.5E−41 | | | | | | | | |
| UBE2T | protein_coding | | | −1.37 | 0.04 | | | | | | | | |
| SLC22A23 | protein_coding | | | −0.96 | 0.024 | | | | | | | | |
| NUDT6 | protein_coding | | | −1.43 | 0.015 | | | | | | | | |
| GSPT2 | protein_coding | | | −1.48 | 0.0091 | | | | | | | | |
| EMILIN2 | protein_coding | | | 0.78 | 0.038 | | | | | | | | |
| CXCR1 | protein_coding | | | −0.94 | 0.041 | | | | | | | | |
| MRC1 | protein_coding | | | −0.89 | 0.045 | | | | | | | | |
| F2RL3 | protein_coding | | | −0.71 | 0.0013 | | | | | | | | |
| ZBTB3 | protein_coding | | | −1.6 | 0.046 | | | | | | | | |
| LRRC61 | protein_coding | | | −1.61 | 0.022 | | | | | | | | |
| NUDT7 | protein_coding | | | −1.61 | 0.042 | | | | | | | | |
| EYS | protein_coding | | | 1.7 | 0.022 | | | | | | | | |
| ZNF81 | protein_coding | | | −1.64 | 0.01 | | | | | | | | |
| ZNF778 | protein_coding | | | −1.67 | 0.029 | | | | | | | | |
| SLCO5A1 | protein_coding | | | −0.68 | 1.2E−33 | | | | | | | | |
| SWT1 | protein_coding | | | −1.79 | 0.013 | | | | | | | | |
| RACGAP1 | protein_coding | | | −1.84 | 0.0009 | | | | | | | | |
| MAPK11 | protein_coding | | | −1.88 | 0.0098 | | | | | | | | |
| ZBTB47 | protein_coding | | | −1.9 | 0.000027 | | | | | | | | |
| MYPOP | protein_coding | | | 1.91 | 0.0046 | | | | | | | | |
| ZNRF3 | protein_coding | | | 0.81 | 0.037 | | | | | | | | |
| PO4 | protein_coding | | | −1.94 | 0.022 | | | | | | | 1.86 | 0.029 |
| ITGB3BP | protein_coding | | | −2.28 | 0.00029 | | | | | | | | |
| SMIM18 | protein_coding | | | 1.74 | 0.035 | | | | | | | | |
| CACNA1F | protein_coding | −0.7 | 0.039 | | | | | | | | | | |
| CEP112 | protein_coding | 0.78 | 0.033 | | | | | | | | | | |
| AP3B2 | protein_coding | 0.77 | 0.012 | | | | | | | | | | |
| CLEC4F | protein_coding | 0.69 | 0.04 | | | | | | | | | | |
| ITPRIPL1 | protein_coding | 0.92 | 0.036 | | | | | | | | | | |
| WDR5B | protein_coding | −0.74 | 0.029 | | | | | | | | | | |
| IQCC | protein_coding | −0.81 | 0.029 | | | | | | | | | | |
| DHDH | protein_coding | −0.86 | 0.036 | | | | | | | | | | |
| AASS | protein_coding | | | | | | | | | | | | |
| CNIH2 | protein_coding | | | | | | | | | | | | |
| HCN2 | protein_coding | | | | | | | | | | | | |
| IL10RB | protein_coding | | | | | | | | | | | | |
| DMXL2 | protein_coding | | | | | | | | | | | | |
| PAQR4 | protein_coding | | | | | | | | | | | | |
| AC104581.1 | protein_coding | | | | | | | | | | | | |
| ACACA | protein_coding | | | | | | | | | | | | |
| REG4 | protein_coding | | | | | | | | | | | | |
| ADGRB2 | protein_coding | | | | | | | | | | | | |
| ACRBP | protein_coding | | | | | | | | | | | | |
| ACSM3 | protein_coding | | | | | | | | | | | | |
| ACTN1 | protein_coding | | | | | | | | | | | | |
| KDELR1 | protein_coding | | | | | | | | | | | | |
| PLOD3 | protein_coding | | | | | | | | | | | | |
| RETREG3 | protein_coding | | | | | | | | | | | | |
| TACR2 | protein_coding | | | | | | | | | | | | |
| TMEM203 | protein_coding | | | | | | | | | | | | |
| VAMP4 | protein_coding | | | | | | | | | | | | |
| ADM5 | protein_coding | | | | | | | | | | | | |
| AFAP1L2 | protein_coding | | | | | | | | | | | | |
| AIF1 | protein_coding | | | | | | | | | | | | |
| RNF5 | protein_coding | | | | | | | | | | | | |
| AK5 | protein_coding | | | | | | | | | | | | |
| AKAP3 | protein_coding | | | | | | | | | | | −2.58 | 0.0057 |
| THAP4 | protein_coding | | | | | | | | | | | | |
| ALDH3B1 | protein_coding | | | | | | | −1.68 | 0.029 | | | −2.48 | 0.000091 |
| ALG13 | protein_coding | | | | | | | | | | | | |
| ALOX5 | protein_coding | | | | | | | | | | | | |
| AMACR | protein_coding | | | | | | | | | | | | |
| AMER1 | protein_coding | | | | | | | | | | | | |
| ANKRD44 | protein_coding | | | | | | | | | | | | |
| TMEM179B | protein_coding | | | | | | | | | | | | |
| APOBEC3A | protein_coding | | | | | | | | | | | | |
| KDELR2 | protein_coding | | | | | | | | | | | | |
| KCNQ4 | protein_coding | | | | | | | | | | | | |
| ARHGAP33 | protein_coding | | | | | | | | | | | | |
| ARMC9 | protein_coding | | | | | | | | | | | | |
| FPR1 | protein_coding | | | | | | | | | | | | |
| G0S2 | protein_coding | | | | | | | | | | | | |
| ASXL2 | protein_coding | | | | | | | | | | | | |
| ATIC | protein_coding | | | | | | | | | | | −1.06 | 0.034 |
| CALCRL | protein_coding | | | | | | | | | | | | |
| B3GNT5 | protein_coding | | | | | | | | | | | | |
| B4GALNT1 | protein_coding | | | | | | | | | | | | |
| BAAT | protein_coding | | | | | | | | | | | | |
| BAIAP2L1 | protein_coding | | | | | | | | | | | | |
| BATF3 | protein_coding | | | | | | | | | | | | |
| TNFSF13B | protein_coding | | | | | | | | | | | | |
| BCL7A | protein_coding | | | | | | | | | | | | |
| STK36 | protein_coding | | | | | | | | | | | | |
| BLK | protein_coding | | | | | | | | | | | | |
| BTBD19 | protein_coding | | | | | | | | | | | | |
| BTBD6 | protein_coding | | | | | | | | | | | | |
| BTBD9 | protein_coding | | | | | | | | | | | | |
| BTK | protein_coding | | | | | | | | | | | | |
| ZDHHC14 | protein_coding | | | | | | | | | | | | |
| C16orf71 | protein_coding | | | | | | | | | | | | |
| C16orf86 | protein_coding | | | | | | | | | | | | |
| CTLA4 | protein_coding | | | | | | | | | | | | |
| C17orf98 | protein_coding | | | | | | | | | | | −0.69 | 0.0000026 |
| PCYOX1L | protein_coding | | | | | | | | | | | | |
| C2CD2 | protein_coding | | | | | | | | | | | −1.13 | 0.017 |
| C4orf19 | protein_coding | | | | | | | | | | | | |
| C4orf33 | protein_coding | | | | | | | | | | | | |
| LGALS3BP | protein_coding | | | | | | | | | | | | |
| C8orf46 | protein_coding | | | | | | | | | | | | |
| C9orf40 | protein_coding | | | | | | | | | | | | |
| EPHB3 | protein_coding | | | | | | | | | | | | |
| TSSK4 | protein_coding | | | | | | | | | | | | |
| SEMA6B | protein_coding | | | | | | | | | | | | |
| RGMB | protein_coding | | | | | | | | | | | | |
| CAMSAP1 | protein_coding | | | | | | | | | | | | |
| CAPN8 | protein_coding | | | | | | | | | | | | |
| CARD6 | protein_coding | | | | | | | | | | | | |
| CD300C | protein_coding | | | | | | | | | | | | |
| CASP6 | protein_coding | | | | | | | | | | | | |
| CBX8 | protein_coding | | | | | | | | | | | | |
| SLC19A1 | protein_coding | | | | | | | | | | | | |
| GPM6B | protein_coding | | | | | | | | | | | | |
| CCDC184 | protein_coding | | | | | | | | | | | −0.93 | 0.014 |
| CCM2 | protein_coding | | | | | | | | | | | | |
| CCNB1 | protein_coding | | | | | | | −1.75 | 0.044 | | | | |
| LHFPL2 | protein_coding | | | | | | | | | | | | |
| FCN1 | protein_coding | | | | | | | | | | | | |
| SV2A | protein_coding | | | | | | | | | | | | |
| MCEMP1 | protein_coding | | | | | | | | | | | | |
| IL10 | protein_coding | | | | | | | | | | | | |
| KCNH3 | protein_coding | | | | | | | | | | | | |
| TTYH3 | protein_coding | | | | | | | | | | | | |
| TMEM170B | protein_coding | | | | | | | | | | | | |
| FAM98B | protein_coding | | | | | | | | | | | | |
| NTSR1 | protein_coding | | | | | | | | | | | | |
| CDCA7 | protein_coding | | | | | | | | | | | | |
| SLC24A4 | protein_coding | | | | | | | | | | | | |
| SIGLEC14 | protein_coding | | | | | | | −0.93 | 0.000011 | | | −1.86 | 3.7E−09 |
| CEBPD | protein_coding | | | | | | | | | | | | |
| PGLYRP2 | protein_coding | | | | | | | | | | | | |
| CEP83 | protein_coding | | | | | | | | | | | | |
| OR56B1 | protein_coding | | | | | | | | | | | | |
| CFAP70 | protein_coding | | | | | | | | | | | | |
| CHAMP1 | protein_coding | | | | | | | | | | | | |
| CHD6 | protein_coding | | | | | | | | | | | | |
| FNDC10 | protein_coding | | | | | | | | | | | | |
| CACFD1 | protein_coding | | | | | | | | | | | | |
| FITM2 | protein_coding | | | | | | | | | | | | |
| ASIC3 | protein_coding | | | | | | | | | | | | |
| AC007040.2 | protein_coding | | | | | | | | | | | | |
| LRRC3 | protein_coding | | | −0.75 | 0.012 | −0.71 | 0.0014 | | | | | | |
| CMBL | protein_coding | | | | | | | | | | | | |
| CNFN | protein_coding | | | | | | | | | | | | |
| FANCA | protein_coding | | | | | | | | | | | | |
| OR1L8 | protein_coding | | | | | | | | | | | | |
| ASTL | protein_coding | | | | | | | | | | | | |
| S100A8 | protein_coding | | | | | | | | | | | | |
| MTRNR2L3 | protein_coding | | | | | | | | | | | | |
| S100A9 | protein_coding | | | | | | | | | | | | |
| COQ9 | protein_coding | | | | | | | | | | | | |
| IL17C | protein_coding | | | | | | | | | | | | |
| CRAMP1 | protein_coding | | | | | | | | | | | 1.96 | 0.047 |
| CNTNAP1 | protein_coding | | | | | | | | | | | | |
| MEGF8 | protein_coding | | | | | | | | | | | | |
| PRSS22 | protein_coding | | | | | | | | | | | | |
| CRYBB2 | protein_coding | | | | | | | | | | | −1.3 | 1.8E−20 |
| MFSD6L | protein_coding | | | | | | | | | | | | |
| CFP | protein_coding | | | | | | | | | | | | |
| SLC35G5 | protein_coding | | | | | | | | | | | | |
| LYZ | protein_coding | | | | | | | | | | | | |
| MMP17 | protein_coding | | | | | | | | | | | | |
| LAPTM4B | protein_coding | | | | | | | | | | | | |
| PRRG4 | protein_coding | | | | | | | | | | | | |
| CYGB | protein_coding | | | | | | | | | | | −0.63 | 0.00077 |
| SLC1A2 | protein_coding | | | | | | | | | | | | |
| MT-ND1 | protein_coding | | | | | | | | | | | | |
| QPCT | protein_coding | | | | | | | | | | | | |
| SEMA3B | protein_coding | | | | | | | | | | | | |
| PLXNA4 | protein_coding | | | | | | | | | | | | |
| CYB561D1 | protein_coding | | | | | | | | | | | | |
| DDN | protein_coding | | | | | | | | | | | | |
| CCR8 | protein_coding | | | | | | | | | | | | |
| ATP6V0A1 | protein_coding | | | | | | | | | | | | |
| DHRS12 | protein_coding | | | | | | | | | | | | |
| C8G | protein_coding | | | | | | | | | | | | |
| TMEM243 | protein_coding | | | | | | | | | | | | |
| DNA2 | protein_coding | | | | | | | | | | | | |
| PGAP3 | protein_coding | | | | | | | | | | | | |
| DNMT3B | protein_coding | | | | | | | | | | | −0.67 | 0.000011 |
| DOCK10 | protein_coding | | | | | | | | | | | | |
| FAR2 | protein_coding | | | | | | | | | | | | |
| MARCO | protein_coding | | | | | | | | | | | | |
| DTX1 | protein_coding | | | | | | | | | | | | |
| CDH5 | protein_coding | | | | | | | | | | | | |
| DUSP28 | protein_coding | | | | | | | | | | | | |
| DUSP6 | protein_coding | | | | | | | | | | | | |
| DZANK1 | protein_coding | | | | | | | | | | | | |
| CLCN1 | protein_coding | | | | | | | | | | | | |
| EFCAB12 | protein_coding | | | | | | | | | | | | |
| EFHC2 | protein_coding | | | | | | | | | | | 1.45 | 0.026 |
| EIF1AX | protein_coding | | | | | | | | | | | | |
| DPP4 | protein_coding | | | | | | | | | | | | |
| COL1A1 | protein_coding | | | | | | | | | | | | |
| EML6 | protein_coding | | | | | | | | | | | | |
| ENO4 | protein_coding | | | | | | | | | | | | |
| ENOX2 | protein_coding | | | | | | | | | | | | |
| EPS8L1 | protein_coding | | | | | | | | | | | | |
| EXOC6B | protein_coding | | | | | | | | | | | | |
| GLT1D1 | protein_coding | | | | | | | | | | | | |
| ATP8B3 | protein_coding | | | | | | | | | | | | |
| B3GALT2 | protein_coding | | | | | | | | | | | | |
| CES4A | protein_coding | | | | | | | | | | | | |
| FAM109B | protein_coding | | | | | | | | | | | | |
| FAM161B | protein_coding | | | | | | | | | | | | |
| GPR75 | protein_coding | | | | | | | | | | | | |
| DRD3 | protein_coding | | | | | | | | | | | | |
| FAM212B | protein_coding | | | | | | | | | | | | |
| KIAA0319L | protein_coding | | | | | | | | | | | | |
| FAM216A | protein_coding | | | | | | | | | | | | |
| FAM227B | protein_coding | | | | | | | | | | | | |
| RMND1 | protein_coding | | | | | | | | | | | | |
| SLC38A7 | protein_coding | | | | | | | | | | | | |
| FANCL | protein_coding | | | | | | | | | | | | |
| MS4A6A | protein_coding | | | | | | | | | | | | |
| COL9A2 | protein_coding | | | | | | | | | | | | |
| FBP1 | protein_coding | | | | | | | | | | | | |
| FBXO2 | protein_coding | | | | | | | | | | | | |
| CHIC1 | protein_coding | | | | | | | | | | | | |
| FDXR | protein_coding | | | | | | | | | | | | |
| MBOAT2 | protein_coding | | | | | | | | | | | | |
| LYSMD4 | protein_coding | | | | | | | | | | | | |
| TPCN1 | protein_coding | | | | | | | | | | | | |
| FTCDNL1 | protein_coding | | | | | | | | | | | | |
| TBXAS1 | protein_coding | | | | | | | | | | | | |
| TVP23C | protein_coding | | | | | | | | | | | | |
| FOSL1 | protein_coding | | | | | | | | | | | | |
| FOXRED2 | protein_coding | | | | | | | | | | | | |
| SPON1 | protein_coding | | | | | | | | | | | | |
| TMEM238 | protein_coding | | | | | | | | | | | | |
| CDHR1 | protein_coding | | | | | | | | | | | | |
| IL12A | protein_coding | | | | | | | | | | | | |
| KCNQ5 | protein_coding | | | | | | | | | | | | |
| FXYD2 | protein_coding | | | | | | | | | | | | |
| GBGT1 | protein_coding | | | | | | | | | | | 0.63 | 0.022 |
| GCA | protein_coding | | | | | | | | | | | | |
| GCAT | protein_coding | | | | | | | −0.73 | 0.017 | | | | |
| SLC25A17 | protein_coding | | | | | | | | | | | | |
| SLC4A8 | protein_coding | | | | | | | | | | | | |
| GFRA2 | protein_coding | | | | | | | | | | | | |
| GGACT | protein_coding | | | | | | | | | | | | |
| GGCT | protein_coding | | | | | | | | | | | 1.45 | 0.039 |
| GINS3 | protein_coding | | | | | | | | | | | 2.03 | 0.001 |
| GIPC3 | protein_coding | | | | | | | | | | | | |
| GIPR | protein_coding | | | | | | | | | | | | |
| F5 | protein_coding | | | | | | | | | | | | |
| GNG12 | protein_coding | | | | | | | | | | | | |
| GPATCH2L | protein_coding | | | | | | | | | | | | |
| PODXL | protein_coding | | | | | | | | | | | | |
| SLC25A42 | protein_coding | | | | | | | | | | | | |
| SEMA6C | protein_coding | | | | | | | | | | | | |
| CD320 | protein_coding | | | | | | | | | | | | |
| RDM1 | protein_coding | | | | | | | | | | | | |
| GPRIN1 | protein_coding | | | | | | | | | | | | |
| GRASP | protein_coding | | | | | | | | | | | | |
| NOTCH4 | protein_coding | | | | | | | | | | | | |
| GSTM2 | protein_coding | | | | | | | | | | | | |
| MAOA | protein_coding | | | | | | | | | | | | |
| HADH | protein_coding | | | | | | | | | | | | |
| HARBI1 | protein_coding | | | | | | | | | | | | |
| C14orf132 | protein_coding | | | | | | | | | | | | |
| HCFC1 | protein_coding | | | | | | | | | | | −1.78 | 0.0046 |
| SYT6 | protein_coding | | | | | | | | | | | | |
| HDAC9 | protein_coding | | | | | | | | | | | −2.55 | 0.012 |
| TMPRSS2 | protein_coding | | | | | | | | | | | | |
| MEMO1 | protein_coding | | | | | | | | | | | | |
| HIST1H2AE | protein_coding | | | | | | | | | | | | |
| HIST1H2BF | protein_coding | | | | | | | | | | | | |
| HIST1H2BM | protein_coding | | | | | | | | | | | | |
| HIST1H3E | protein_coding | | | | | | | | | | | | |
| HIST1H3I | protein_coding | | | | | | | | | | | | |
| HIST1H4E | protein_coding | | | | | | | | | | | | |
| HIST1H4I | protein_coding | | | | | | | | | | | | |
| HLF | protein_coding | | | | | | | | | | | | |
| BIK | protein_coding | | | −1.43 | 0.024 | | | | | | | | |
| HOMER1 | protein_coding | | | | | | | | | | | | |
| HOOK1 | protein_coding | | | | | | | | | | | | |
| HOXA1 | protein_coding | | | | | | | | | | | | |
| HSD17B6 | protein_coding | | | | | | | | | | | | |
| HSD17B7 | protein_coding | | | | | | | | | | | | |
| HSPA13 | protein_coding | | | | | | | | | | | | |
| IFI44L | protein_coding | | | | | | | | | | | | |
| IFT140 | protein_coding | | | | | | | | | | | | |
| IFT172 | protein_coding | | | | | | | | | | | −1.56 | 0.027 |
| SDR42E2 | protein_coding | | | | | | | | | | | | |
| SEMA3G | protein_coding | | | | | | | | | | | −2.3 | 0.0034 |
| CCDC136 | protein_coding | | | | | | | | | | | | |
| APOO | protein_coding | | | | | | | | | | | | |
| IMPACT | protein_coding | | | | | | | | | | | | |
| ISPD | protein_coding | | | | | | | | | | | | |
| ITPKC | protein_coding | | | | | | | | | | | | |
| SEMA6A | protein_coding | | | | | | | | | | | | |
| JSRP1 | protein_coding | | | | | | | | | | | | |
| JUP | protein_coding | | | | | | | | | | | | |
| KBTBD8 | protein_coding | | | | | | | | | | | | |
| LTC4S | protein_coding | | | | | | | | | | | | |
| PTPRS | protein_coding | | | | | | | | | | | | |
| PLPP1 | protein_coding | | | | | | | | | | | | |
| PLXDC2 | protein_coding | | | | | | | | | | | | |
| CLN6 | protein_coding | | | | | | | | | | | | |
| MT-ND3 | protein_coding | | | | | | | | | | | | |
| NDFIP2 | protein_coding | | | | | | | | | | | | |
| METTL7A | protein_coding | | | | | | | | | | | | |
| KDM8 | protein_coding | | | | | | | | | | | | |
| CCDC163 | protein_coding | | | | | | | | | | | | |
| KIAA0825 | protein_coding | | | | | | | | | | | | |
| KIF1BP | protein_coding | | | | | | | | | | | | |
| KIF24 | protein_coding | | | | | | | | | | | | |
| KIF5C | protein_coding | | | | | | | | | | | | |
| NEMP1 | protein_coding | | | | | | | | | | | | |
| KMT2D | protein_coding | | | | | | | | | | | | |
| L3HYPDH | protein_coding | | | | | | | | | | | | |
| CXADR | protein_coding | | | | | | | | | | | | |
| LANCL3 | protein_coding | | | | | | | | | | | | |
| ANO6 | protein_coding | | | | | | | | | | | | |
| LENG9 | protein_coding | | | | | | | | | | | | |
| CYTL1 | protein_coding | | | | | | | | | | | −0.6 | 0.000031 |
| LGMN | protein_coding | | | | | | | | | | | | |
| SLC37A4 | protein_coding | | | | | | | | | | | | |
| NPHP4 | protein_coding | | | | | | | | | | | −2.63 | 0.02 |
| PTGIR | protein_coding | | | | | | | | | | | −2.63 | 0.0069 |
| LONRF3 | protein_coding | | | | | | | | | | | | |
| ZACN | protein_coding | | | | | | | −1.76 | 0.00024 | | | −2.41 | 0.0047 |
| LRRC75B | protein_coding | | | | | | | | | | | | |
| RPAP1 | protein_coding | | | | | | | | | | | −2.1 | 0.044 |
| LYPD2 | protein_coding | | | | | | | | | | | | |
| INSL4 | protein_coding | | | | | | | | | | | 0.69 | 0.00058 |
| MAFK | protein_coding | | | | | | | | | | | | |
| MAML3 | protein_coding | | | | | | | | | | | | |
| MAMLD1 | protein_coding | | | | | | | | | | | | |
| MAP2K6 | protein_coding | | | | | | | | | | | | |
| MAP3K21 | protein_coding | | | | | | | | | | | | |
| MAP4K4 | protein_coding | | | | | | | | | | | | |
| MAPK15 | protein_coding | | | | | | | | | | | | |
| MAPK8IP1 | protein_coding | | | | | | | | | | | | |
| PRUNE2 | protein_coding | | | | | | | | | | | −1.01 | 3E−11 |
| MARS2 | protein_coding | | | | | | | | | | | | |
| TREML1 | protein_coding | | | | | | | | | | | −1 | 0.0015 |
| CCR3 | protein_coding | | | | | | | | | | | −0.91 | 8.8E−12 |
| VSIG4 | protein_coding | | | | | | | | | | | −0.86 | 2.3E−16 |
| MSLN | protein_coding | | | | | | | | | | | −0.85 | 2.1E−11 |
| TUBA8 | protein_coding | | | | | | | | | | | −0.78 | 4.8E−14 |
| DUOXA1 | protein_coding | | | | | | | | | | | −0.67 | 0.00038 |
| MGLL | protein_coding | | | | | | | | | | | | |
| FXYD6 | protein_coding | | | | | | | | | | | −0.61 | 8.6E−11 |
| MKRN3 | protein_coding | | | | | | | | | | | | |
| MORN4 | protein_coding | | | | | | | | | | | | |
| MROH8 | protein_coding | | | | | | | | | | | | |
| MRPL34 | protein_coding | | | | | | | | | | | | |
| MSRB3 | protein_coding | | | | | | | | | | | | |
| MT2A | protein_coding | | | | | | | | | | | | |
| MTSS1L | protein_coding | | | | | | | | | | | | |
| MTX2 | protein_coding | | | | | | | | | | | | |
| MYCBP2 | protein_coding | | | | | | | | | | | | |
| MYLPF | protein_coding | | | | | | | | | | | | |
| MYOM2 | protein_coding | | | | | | | | | | | | |
| NAF1 | protein_coding | | | | | | | | | | | | |
| NAPA | protein_coding | | | | | | | | | | | −0.69 | 0.049 |
| NEK1 | protein_coding | | | | | | | | | | | | |
| NHLH2 | protein_coding | | | | | | | | | | | | |
| NPM2 | protein_coding | | | | | | | | | | | −2.81 | 0.04 |
| NSUN4 | protein_coding | | | | | | | | | | | | |
| NUBP2 | protein_coding | | | | | | | | | | | | |
| NUDT18 | protein_coding | | | | | | | | | | | | |
| OGG1 | protein_coding | | | | | | | | | | | | |
| OLIG1 | protein_coding | | | | | | | | | | | | |
| OPHN1 | protein_coding | | | | | | | | | | | | |
| OTUD7B | protein_coding | | | | | | | | | | | −1.65 | 0.016 |
| OVGP1 | protein_coding | | | | | | | | | | | | |
| PAFAH1B3 | protein_coding | | | | | | | | | | | | |
| PAH | protein_coding | | | | | | | | | | | | |
| PARG | protein_coding | | | | | | | −1.51 | 0.013 | | | | |
| PARS2 | protein_coding | | | | | | | | | | | | |
| PCCA | protein_coding | | | | | | | | | | | | |
| PELP1 | protein_coding | | | | | | | | | | | | |
| PGM2 | protein_coding | | | | | | | | | | | | |
| PHLPP1 | protein_coding | | | | | | | | | | | | |
| PIFO | protein_coding | | | | | | | | | | | | |
| PLEKHB1 | protein_coding | | | | | | | | | | | | |
| PLK1 | protein_coding | | | | | | | | | | | −2.03 | 0.042 |
| PLK4 | protein_coding | | | | | | | | | | | | |
| PMS2 | protein_coding | | | | | | | | | | | | |
| PNKP | protein_coding | | | | | | | | | | | −1.54 | 0.021 |
| POLD1 | protein_coding | | | | | | | | | | | | |
| PPL | protein_coding | | | | | | | | | | | | |
| PPM1H | protein_coding | | | | | | | | | | | | |
| PPP4R1 | protein_coding | | | | | | | | | | | | |
| PRC1 | protein_coding | | | | | | | | | | | 1.59 | 0.05 |
| PRDM13 | protein_coding | | | | | | | | | | | | |
| PRKD2 | protein_coding | | | | | | | | | | | | |
| PRR34 | protein_coding | | | | | | | | | | | | |
| GREM2 | protein_coding | | | | | | | −1.55 | 0.00016 | | | | |
| PSMA8 | protein_coding | | | | | | | | | | | | |
| PSRC1 | protein_coding | | | | | | | | | | | | |
| PUS3 | protein_coding | | | | | | | | | | | | |
| PYCR3 | protein_coding | | | | | | | | | | | | |
| RAB26 | protein_coding | | | | | | | | | | | −1.45 | 0.0092 |
| RAD54L | protein_coding | | | | | | | | | | | | |
| RALGPS2 | protein_coding | | | | | | | | | | | | |
| RASGRF2 | protein_coding | | | | | | | | | | | | |
| RASL11A | protein_coding | | | | | | | | | | | | |
| RBFA | protein_coding | | | | | | | | | | | | |
| RBMS2 | protein_coding | | | | | | | | | | | | |
| REXO5 | protein_coding | | | | | | | | | | | | |
| RIMS3 | protein_coding | | | | | | | | | | | | |
| RNF141 | protein_coding | | | | | | | | | | | | |
| RPL10A | protein_coding | | | | | | | | | | | | |
| RPL34 | protein_coding | | | | | | | | | | | | |
| RPL37 | protein_coding | | | | | | | | | | | | |
| RPL6 | protein_coding | | | | | | | | | | | | |
| RPP30 | protein_coding | | | | | | | | | | | | |
| RPS21 | protein_coding | | | | | | | | | | | | |
| RPS24 | protein_coding | | | | | | | | | | | | |
| RSPH1 | protein_coding | | | | | | | | | | | | |
| RSPH9 | protein_coding | | | | | | | | | | | −2.34 | 0.044 |
| RTN4IP1 | protein_coding | | | | | | | | | | | | |
| SAFB | protein_coding | | | | | | | | | | | −0.77 | 0.039 |
| SASH3 | protein_coding | | | | | | | | | | | | |
| SCML1 | protein_coding | | | | | | | | | | | | |
| SCML4 | protein_coding | | | | | | | | | | | | |
| SCRIB | protein_coding | | | | | | | | | | | | |
| SCYL1 | protein_coding | | | | | | | | | | | | |
| SGK3 | protein_coding | | | | | | | | | | | | |
| SH2B2 | protein_coding | | | | | | | | | | | | |
| SH2D7 | protein_coding | | | | | | | | | | | −0.62 | 0.00000047 |
| SLC25A30 | protein_coding | | | | | | | | | | | −1.96 | 0.017 |
| SMARCD3 | protein_coding | | | | | | | | | | | | |
| SMC1A | protein_coding | | | | | | | | | | | | |
| SOBP | protein_coding | | | | | | | | | | | | |
| SOCS6 | protein_coding | | | | | | | | | | | | |
| SORD | protein_coding | | | | | | | | | | | | |
| SOWAHD | protein_coding | | | | | | | | | | | −1.25 | 1.9E−17 |
| SOX12 | protein_coding | | | | | | | 1.04 | 0.000012 | | | 2.21 | 0.000034 |
| SPAG1 | protein_coding | | | | | | | | | | | | |
| SPRN | protein_coding | | | | | | | | | | | | |
| CYP2U1 | protein_coding | | | | | | | −1.46 | 0.048 | | | | |
| MPIG6B | protein_coding | | | | | | | −0.73 | 0.0017 | | | | |
| STARD5 | protein_coding | | | | | | | | | | | | |
| STK19 | protein_coding | | | | | | | | | | | | |
| STPG1 | protein_coding | | | | | | | | | | | | |
| SUV39H2 | protein_coding | | | | | | | | | | | | |
| SYK | protein_coding | | | | | | | | | | | −1.63 | 0.031 |
| SZT2 | protein_coding | | | | | | | | | | | | |
| TAF1A | protein_coding | | | | | | | | | | | | |
| TBC1D4 | protein_coding | | | | | | | | | | | | |
| TBCK | protein_coding | | | | | | | | | | | | |
| TBX3 | protein_coding | | | | | | | | | | | | |
| TEP1 | protein_coding | | | | | | | | | | | | |
| TET3 | protein_coding | | | | | | | | | | | | |
| TFAP2E | protein_coding | | | | | | | | | | | | |
| TIAM1 | protein_coding | | | | | | | | | | | | |
| TMEM256- | protein_coding | | | | | | | | | | | | |
| PLSCR3 | | | | | | | | | | | | | |
| TRIM58 | protein_coding | | | | | | | | | | | | |
| TSR2 | protein_coding | | | | | | | | | | | | |
| TTLL5 | protein_coding | | | | | | | | | | | | |
| TUBB | protein_coding | | | | | | | | | | | | |
| UCHL3 | protein_coding | | | | | | | | | | | 1.44 | 0.027 |
| UPF3A | protein_coding | | | | | | | | | | | | |
| USP40 | protein_coding | | | | | | | | | | | | |
| VPS50 | protein_coding | | | | | | | | | | | | |
| WASF1 | protein_coding | | | | | | | | | | | | |
| WDR44 | protein_coding | | | | | | | | | | | | |
| WDR86 | protein_coding | | | | | | | | | | | | |
| ZBTB10 | protein_coding | | | | | | | | | | | 1.7 | 0.013 |
| ZKSCAN4 | protein_coding | | | | | | | | | | | | |
| ZNF138 | protein_coding | | | | | | | | | | | | |
| ZNF20 | protein_coding | | | | | | | | | | | | |
| ZNF23 | protein_coding | | | | | | | | | | | | |
| ZNF257 | protein_coding | | | | | | | | | | | | |
| ZNF280B | protein_coding | | | | | | | | | | | | |
| ZNF304 | protein_coding | | | | | | | | | | | | |
| ZNF318 | protein_coding | | | | | | | | | | | 1.22 | 0.035 |
| ZNF324B | protein_coding | | | | | | | | | | | | |
| ZNF460 | protein_coding | | | | | | | | | | | | |
| ZNF544 | protein_coding | | | | | | | | | | | | |
| ZNF599 | protein_coding | | | | | | | | | | | | |
| ZNF630 | protein_coding | | | | | | | | | | | | |
| ZNF646 | protein_coding | | | | | | | | | | | −1.38 | 0.049 |
| ZNF726 | protein_coding | | | | | | | | | | | 1.9 | 0.012 |
| ZNF736 | protein_coding | | | | | | | | | | | | |
| ZNF841 | protein_coding | | | | | | | | | | | | |
| ZNF93 | protein_coding | | | | | | | | | | | 1.56 | 0.03 |
| ZNHIT1 | protein_coding | | | | | | | | | | | | |
| ZSWIM6 | protein_coding | | | | | | | | | | | | |
| | Total number of genes | 18 | | 90 | | 65 | | 11 | | 304 | | 172 | |

TABLE 3
CD8 memory

| | | CD8 memory | | | | | |
|---|---|---|---|---|---|---|---|
| | | PD vs HC | | PD_R vs PD_NR | | PD_R vs HC_NR | |
| | | log2 fold | adj p | log2 fold | adj p | log2 fold | adj p |
| Gene | gene type | change | value | change | value | change | value |
| SLC16A13 | protein_coding | | | | | | |
| POU5F2 | protein_coding | | | | | | |
| TBC1D8 | protein_coding | | | | | | |
| C11orf65 | protein_coding | | | | | | |
| CX3CR1 | protein_coding | | | | | | |
| FCGBP | protein_coding | | | | | | |
| TNFAIP8L2 | protein_coding | | | | | | |
| CSF3R | protein_coding | | | | | | |
| XPNPEP2 | protein_coding | | | | | | |
| PRAG1 | protein_coding | | | | | | |
| CD8A | protein_coding | | | | | | |
| ARHGEF5 | protein_coding | | | | | | |
| SIGLEC7 | protein_coding | | | | | | |
| ZNF546 | protein_coding | | | | | | |
| AGAP6 | protein_coding | | | | | | |
| SRC | protein_coding | | | 1.2 | 0.00029 | 1.18 | 0.00074 |
| RAB20 | protein_coding | | | | | | |
| AC051649.2 | protein_coding | | | | | | |
| DOK6 | protein_coding | | | | | | |
| CALHM2 | protein_coding | | | | | | |
| WIPI1 | protein_coding | | | | | | |
| CORO1C | protein_coding | | | | | | |
| KLHL35 | protein_coding | | | | | | |
| HIST2H2AB | protein_coding | | | | | | |
| E2F2 | protein_coding | | | | | | |
| LAT2 | protein_coding | | | 1.57 | 0.0005 | 1.42 | 0.0097 |
| MAMDC4 | protein_coding | | | | | | |
| TFEB | protein_coding | | | | | | |
| DUSP7 | protein_coding | | | | | | |
| F2RL2 | protein_coding | | | | | | |
| PTPN23 | protein_coding | | | | | | |
| MMP25 | protein_coding | | | | | | |
| CISH | protein_coding | | | | | | |
| PSKH1 | protein_coding | | | | | | |
| TNFAIP2 | protein_coding | | | | | | |
| HS6ST1 | protein_coding | | | | | | |
| PEX26 | protein_coding | | | | | | |
| INMT | protein_coding | | | | | | |
| AP2A1 | protein_coding | | | | | | |
| ZNF175 | protein_coding | | | 0.59 | 0.0022 | | |
| RAB3D | protein_coding | | | | | | |
| CDC42EP2 | protein_coding | | | −1.96 | 0.018 | | |
| FGR | protein_coding | | | | | | |
| FAM131B | protein_coding | | | | | | |
| PRKN | protein_coding | | | 1.54 | 0.00068 | | |
| ADGRG1 | protein_coding | | | | | | |
| ACAN | protein_coding | | | | | | |
| QRICH2 | protein_coding | | | | | | |
| SEPT5 | protein_coding | | | | | | |
| CCNB2 | protein_coding | | | | | | |
| ABCC3 | protein_coding | | | | | | |
| ZNF418 | protein_coding | | | | | | |
| C5orf34 | protein_coding | | | | | | |
| CASP2 | protein_coding | | | | | | |
| FBLN2 | protein_coding | | | | | | |
| MIER2 | protein_coding | | | | | | |
| MICAL3 | protein_coding | | | 2.24 | 0.000054 | 1.86 | 0.008 |
| DHCR24 | protein_coding | | | | | | |
| CD36 | protein_coding | | | | | | |
| PYGO2 | protein_coding | | | | | | |
| GINS1 | protein_coding | | | | | | |
| RAB6B | protein_coding | | | | | | |
| TMEM201 | protein_coding | | | | | | |
| IGFBP6 | protein_coding | | | | | | |
| TPD52 | protein_coding | | | | | | |
| PTGDR2 | protein_coding | | | | | 0.96 | 0.000006 |
| KCNN3 | protein_coding | | | | | | |
| CDA | protein_coding | | | | | | |
| ITGB4 | protein_coding | | | | | | |
| WBP2NL | protein_coding | | | | | | |
| TTC30A | protein_coding | | | | | | |
| ZNF45 | protein_coding | | | | | | |
| CEBPE | protein_coding | | | | | | |
| MRGPRD | protein_coding | | | | | | |
| SPINK4 | protein_coding | | | | | | |
| ABO | protein_coding | | | | | | |
| SELPLG | protein_coding | | | | | | |
| C14orf79 | protein_coding | | | | | | |
| COL16A1 | protein_coding | | | | | | |
| PDZD7 | protein_coding | | | | | | |
| TOM1L1 | protein_coding | | | | | | |
| HHLA2 | protein_coding | | | | | | |
| MFSD8 | protein_coding | | | | | | |
| KCNH4 | protein_coding | | | | | | |
| DCDC2B | protein_coding | | | | | | |
| ST6GALNAC6 | protein_coding | | | | | | |
| TPD52L1 | protein_coding | | | | | | |
| ARRDC5 | protein_coding | | | | | | |
| RAB1B | protein_coding | | | | | | |
| ACAD9 | protein_coding | | | | | | |
| RNF152 | protein_coding | | | | | | |
| ZFHX2 | protein_coding | | | | | | |
| CPB2 | protein_coding | | | | | | |
| CD300LB | protein_coding | | | | | | |
| ZNF620 | protein_coding | | | | | | |
| FAM227A | protein_coding | | | | | | |
| FFAR3 | protein_coding | | | | | | |
| SGCA | protein_coding | | | | | | |
| AL022238.4 | protein_coding | | | | | | |
| PBXIP1 | protein_coding | | | | | | |
| LRFN2 | protein_coding | | | | | | |
| SLC7A8 | protein_coding | | | | | | |
| TAP1 | protein_coding | | | | | | |
| IMPA2 | protein_coding | | | | | | |
| RUFY4 | protein_coding | | | 0.72 | 0.0057 | 0.85 | 0.018 |
| CHRNA10 | protein_coding | | | 1.1 | 0.016 | 0.84 | 0.0088 |
| CA2 | protein_coding | | | | | | |
| EXOC3L2 | protein_coding | | | | | | |
| RET | protein_coding | | | | | | |
| IL22 | protein_coding | | | | | | |
| ST3GAL6 | protein_coding | | | | | | |
| ARHGEF28 | protein_coding | | | | | | |
| SLC15A2 | protein_coding | | | | | | |
| GPR153 | protein_coding | | | | | | |
| TTC26 | protein_coding | | | | | | |
| WEE2 | protein_coding | | | | | | |
| MED15 | protein_coding | | | | | | |
| KIRREL2 | protein_coding | | | | | | |
| MS4A14 | protein_coding | | | | | | |
| CCDC194 | protein_coding | | | 0.7 | 0.0000064 | 0.68 | 0.031 |
| ADGRE5 | protein_coding | | | | | | |
| LRRK2 | protein_coding | | | | | 0.83 | 0.012 |
| SLC30A8 | protein_coding | | | | | | |
| LYPD4 | protein_coding | | | | | | |
| MPO | protein_coding | | | | | | |
| OLFML2B | protein_coding | | | | | | |
| GML | protein_coding | | | | | | |
| FGFBP1 | protein_coding | | | | | | |
| IGDCC4 | protein_coding | | | | | | |
| PPP1R26 | protein_coding | | | | | | |
| MGAM2 | protein_coding | | | | | | |
| HMGCS2 | protein_coding | | | | | | |
| GPR42 | protein_coding | | | | | | |
| ZNF670 | protein_coding | | | | | | |
| KRBOX1 | protein_coding | | | | | | |
| METTL21C | protein_coding | | | | | | |
| LSMEM1 | protein_coding | | | | | | |
| RASD1 | protein_coding | | | | | | |
| LEAP2 | protein_coding | | | | | | |
| AIG1 | protein_coding | | | | | | |
| IL6R | protein_coding | | | | | | |
| ASAH2 | protein_coding | | | | | | |
| GDF11 | protein_coding | | | | | | |
| B3GNT8 | protein_coding | | | | | | |
| STRC | protein_coding | | | | | | |
| ABCD2 | protein_coding | | | | | | |
| CELSR2 | protein_coding | | | | | | |
| SFSWAP | protein_coding | | | | | | |
| ELOA | protein_coding | | | | | | |
| NKAPL | protein_coding | | | | | | |
| TCAIM | protein_coding | | | | | | |
| TOMM5 | protein_coding | | | | | | |
| PTTG1 | protein_coding | | | | | | |
| E2F1 | protein_coding | | | | | | |
| ZNF74 | protein_coding | | | | | | |
| RNASEL | protein_coding | | | | | | |
| PPARGC1B | protein_coding | | | | | | |
| APOL1 | protein_coding | | | | | | |
| PXMP2 | protein_coding | | | | | | |
| ZNF182 | protein_coding | | | 1 | 0.0091 | 0.91 | 0.019 |
| ZFP69B | protein_coding | | | | | | |
| FUT11 | protein_coding | | | | | | |
| EEPD1 | protein_coding | | | | | | |
| INVS | protein_coding | | | | | | |
| P3H4 | protein_coding | | | | | | |
| LARGE2 | protein_coding | | | | | | |
| LAMP3 | protein_coding | | | | | | |
| FAM213A | protein_coding | | | | | | |
| EPHX1 | protein_coding | | | | | | |
| HIST1H4H | protein_coding | | | | | | |
| ECE2 | protein_coding | | | | | | |
| LLGL1 | protein_coding | | | | | | |
| BCL2 | protein_coding | | | | | | |
| ANKRD35 | protein_coding | | | | | | |
| HOOK2 | protein_coding | | | 1.04 | 0.021 | 1.54 | 0.0073 |
| SAMD12 | protein_coding | | | | | | |
| TRPM2 | protein_coding | | | | | | |
| HYLS1 | protein_coding | | | | | | |
| GYPE | protein_coding | | | | | | |
| CD180 | protein_coding | | | | | | |
| CASK | protein_coding | | | | | | |
| TWISTNB | protein_coding | | | | | | |
| CLYBL | protein_coding | | | | | | |
| MTUS1 | protein_coding | | | | | | |
| ROPN1L | protein_coding | | | | | | |
| DCST1 | protein_coding | | | | | | |
| ISM1 | protein_coding | | | | | | |
| ZXDB | protein_coding | | | | | | |
| CSF2RB | protein_coding | | | | | | |
| MPP5 | protein_coding | | | | | | |
| ALCAM | protein_coding | | | | | | |
| ACOT4 | protein_coding | | | | | | |
| TCF19 | protein_coding | | | | | | |
| DOK4 | protein_coding | | | | | | |
| PET100 | protein_coding | | | | | | |
| ZC4H2 | protein_coding | | | | | | |
| LMO7 | protein_coding | | | 2.01 | 0.000094 | | |
| LIPT1 | protein_coding | | | | | | |
| NAP1L2 | protein_coding | | | | | | |
| TMEM97 | protein_coding | | | | | | |
| ELP1 | protein_coding | | | | | | |
| AAMDC | protein_coding | | | | | | |
| SVIL | protein_coding | | | 1.29 | 0.0078 | 1.47 | 0.0041 |
| KNSTRN | protein_coding | | | | | | |
| NSUN6 | protein_coding | | | | | | |
| SARNP | protein_coding | | | 1.63 | 0.03 | | |
| POLA1 | protein_coding | | | | | | |
| RNMT | protein_coding | | | | | | |
| CRB3 | protein_coding | | | | | | |
| PDIA5 | protein_coding | | | | | | |
| ALS2 | protein_coding | | | | | | |
| LYPD8 | protein_coding | | | | | | |
| MYEF2 | protein_coding | | | | | | |
| P3H3 | protein_coding | | | | | | |
| PACRGL | protein_coding | | | | | | |
| H6PD | protein_coding | | | | | | |
| PIGK | protein_coding | | | | | | |
| DBNDD1 | protein_coding | | | | | | |
| CKS1B | protein_coding | | | | | | |
| TTC13 | protein_coding | | | | | | |
| PEX11G | protein_coding | | | | | | |
| BIRC2 | protein_coding | | | | | | |
| NRIP3 | protein_coding | | | | | | |
| COPG2 | protein_coding | | | | | | |
| PPP2R2A | protein_coding | | | | | | |
| HMBOX1 | protein_coding | | | | | | |
| MRPL1 | protein_coding | | | | | | |
| NEK11 | protein_coding | | | | | | |
| SLC7A10 | protein_coding | | | | | | |
| NDUFC1 | protein_coding | | | | | | |
| CHMP5 | protein_coding | | | | | | |
| ZNF805 | protein_coding | | | | | | |
| CREB3L4 | protein_coding | | | | | | |
| IFT22 | protein_coding | | | | | | |
| ADAM22 | protein_coding | | | | | | |
| TAS1R3 | protein_coding | | | | | | |
| PRKAR1B | protein_coding | | | | | | |
| ZNF532 | protein_coding | | | | | | |
| C17orf51 | protein_coding | | | | | | |
| HIST2H2AC | protein_coding | | | | | | |
| SLC35G1 | protein_coding | | | | | | |
| DISC1 | protein_coding | | | | | | |
| MGP | protein_coding | | | | | | |
| PTPDC1 | protein_coding | | | | | | |
| LRRC29 | protein_coding | | | 1.36 | 0.018 | | |
| MTRF1L | protein_coding | | | | | | |
| ZBTB41 | protein_coding | | | | | | |
| FARP1 | protein_coding | | | 1.11 | 0.0077 | 1.93 | 0.000042 |
| TTC12 | protein_coding | | | | | | |
| AC003002.1 | protein_coding | | | | | | |
| PAXIP1 | protein_coding | | | | | | |
| AP2A2 | protein_coding | | | | | | |
| BCKDHB | protein_coding | | | | | | |
| DYNC2H1 | protein_coding | | | | | | |
| NLRP2 | protein_coding | | | | | | |
| NUDT8 | protein_coding | | | | | | |
| CCDC138 | protein_coding | | | | | | |
| ZNF248 | protein_coding | | | | | | |
| SERPINH1 | protein_coding | | | | | | |
| TMEM250 | protein_coding | | | | | | |
| ABAT | protein_coding | | | | | | |
| CPNE2 | protein_coding | | | 0.72 | 0.036 | | |
| SLC25A19 | protein_coding | | | | | | |
| LRRC42 | protein_coding | | | | | | |
| ZCCHC4 | protein_coding | | | | | | |
| GCNT2 | protein_coding | | | | | | |
| GCFC2 | protein_coding | | | | | | |
| ZNF594 | protein_coding | | | | | | |
| CYP2S1 | protein_coding | | | | | | |
| ZFYVE26 | protein_coding | | | | | | |
| BIRC5 | protein_coding | | | | | | |
| SPAG5 | protein_coding | | | | | | |
| ZNF17 | protein_coding | | | | | | |
| CERS6 | protein_coding | | | | | | |
| XXYLT1 | protein_coding | | | 1.15 | 0.038 | | |
| MAN1C1 | protein_coding | | | | | | |
| MYL6B | protein_coding | | | | | | |
| RSPH3 | protein_coding | | | | | | |
| KCNQ1 | protein_coding | | | | | | |
| CYP4V2 | protein_coding | | | | | | |
| DENND1A | protein_coding | | | | | | |
| ACO1 | protein_coding | | | | | | |
| FAM171A1 | protein_coding | | | | | | |
| MAFG | protein_coding | | | | | | |
| MPHOSPH9 | protein_coding | | | | | | |
| FAM19A2 | protein_coding | | | | | | |
| ADCK5 | protein_coding | | | | | | |
| AC069544.2 | protein_coding | | | | | | |
| KLHL32 | protein_coding | | | | | | |
| ZCCHC18 | protein_coding | | | | | | |
| DNAJC25 | protein_coding | | | | | | |
| TCFL5 | protein_coding | | | | | −2.05 | 0.039 |
| PLEKHA7 | protein_coding | | | 1.27 | 0.03 | | |
| HIST2H2BF | protein_coding | | | | | | |
| CNKSR2 | protein_coding | | | | | | |
| CENPE | protein_coding | | | | | | |
| ZNF284 | protein_coding | | | | | | |
| ADCK1 | protein_coding | | | | | | |
| UBAP1L | protein_coding | | | | | | |
| B3GALNT2 | protein_coding | | | | | | |
| NRIP2 | protein_coding | | | | | | |
| MECR | protein_coding | | | | | | |
| BAIAP2 | protein_coding | | | | | | |
| MEGF6 | protein_coding | | | | | | |
| AQP9 | protein_coding | | | | | | |
| SCARA3 | protein_coding | | | | | | |
| SLC35F3 | protein_coding | | | | | | |
| TNFRSF11A | protein_coding | | | | | | |
| TOMM20L | protein_coding | | | | | | |
| GALNT1 | protein_coding | | | | | | |
| HMOX1 | protein_coding | | | 1.73 | 0.0000003 | 2.46 | 0.000000055 |
| MCOLN3 | protein_coding | | | | | | |
| RNF222 | protein_coding | | | | | 0.73 | 0.00000025 |
| CYP2F1 | protein_coding | | | | | | |
| PDPR | protein_coding | | | | | | |
| P2RY6 | protein_coding | | | | | | |
| EIF1AY | protein_coding | | | | | | |
| OGFOD2 | protein_coding | | | | | | |
| METTL16 | protein_coding | | | | | | |
| CCR5 | protein_coding | | | | | | |
| NOP2 | protein_coding | | | | | | |
| PNMA5 | protein_coding | | | | | | |
| CDK17 | protein_coding | | | | | | |
| ETFBKMT | protein_coding | | | | | | |
| ZC3H18 | protein_coding | | | | | | |
| FUT2 | protein_coding | | | | | | |
| SECISBP2L | protein_coding | | | | | | |
| CERK | protein_coding | | | | | | |
| USP2 | protein_coding | | | 1.72 | 0.00000091 | 1.84 | 0.000026 |
| LPIN3 | protein_coding | | | | | | |
| GCM1 | protein_coding | | | | | | |
| PCBP2 | protein_coding | | | | | | |
| ARNTL2 | protein_coding | | | | | | |
| MFSD2B | protein_coding | | | | | | |
| NRP2 | protein_coding | | | | | | |
| ACE | protein_coding | | | | | | |
| CTRC | protein_coding | | | | | | |
| SLC22A16 | protein_coding | | | | | | |
| PTK6 | protein_coding | | | | | | |
| GSC | protein_coding | | | | | | |
| ZNF835 | protein_coding | | | | | | |
| PRSS27 | protein_coding | | | | | | |
| TMTC1 | protein_coding | | | | | | |
| COL4A2 | protein_coding | | | | | | |
| DCHS1 | protein_coding | | | | | | |
| CCR1 | protein_coding | | | 1.15 | 0.000000041 | 1.34 | 0.00000044 |
| SDCBP2 | protein_coding | | | | | | |
| ALG1L2 | protein_coding | | | | | | |
| HFE | protein_coding | | | | | | |
| GLS2 | protein_coding | | | | | | |
| DNM3 | protein_coding | | | | | | |
| FFAR4 | protein_coding | | | | | | |
| FFAR1 | protein_coding | | | | | | |
| HIST1H2AL | protein_coding | | | | | | |
| FSD1 | protein_coding | | | | | | |
| PLPP7 | protein_coding | | | | | | |
| EPHX3 | protein_coding | | | | | | |
| MRPL15 | protein_coding | | | | | | |
| AMOTL1 | protein_coding | | | | | | |
| RPP25 | protein_coding | | | | | | |
| LPAR1 | protein_coding | | | | | | |
| TIGD6 | protein_coding | | | 1.28 | 0.0026 | | |
| CRIM1 | protein_coding | | | | | | |
| PEX3 | protein_coding | | | | | | |
| AL031708.1 | protein_coding | | | | | | |
| SPC24 | protein_coding | | | | | | |
| PRPF40B | protein_coding | | | | | | |
| ADAL | protein_coding | | | | | | |
| PPAT | protein_coding | | | | | | |
| NR1H3 | protein_coding | | | | | | |
| ZNF2 | protein_coding | | | | | | |
| CAD | protein_coding | | | | | | |
| BTBD3 | protein_coding | | | | | | |
| ZNF75D | protein_coding | | | | | | |
| ZSCAN9 | protein_coding | | | | | | |
| GCNT7 | protein_coding | | | | | | |
| ACTA1 | protein_coding | | | | | | |
| POPDC2 | protein_coding | | | | | | |
| NAPSA | protein_coding | | | | | | |
| SNX32 | protein_coding | | | | | | |
| CTDSPL | protein_coding | | | | | | |
| SRGAP2 | protein_coding | | | | | | |
| SPOCD1 | protein_coding | | | | | | |
| CLPX | protein_coding | | | | | | |
| ZNF700 | protein_coding | | | | | | |
| GPR171 | protein_coding | | | | | | |
| POLR3E | protein_coding | | | | | | |
| NUDT4 | protein_coding | | | | | | |
| DKK3 | protein_coding | | | | | | |
| TPR | protein_coding | | | | | | |
| MATR3 | protein_coding | | | | | | |
| ZNF385C | protein_coding | | | | | | |
| HIGD1A | protein_coding | | | | | | |
| KDF1 | protein_coding | | | | | | |
| AATK | protein_coding | | | | | | |
| ZC3H12C | protein_coding | | | | | | |
| SLC45A4 | protein_coding | | | | | | |
| CLIP3 | protein_coding | | | | | | |
| FAM173B | protein_coding | | | | | | |
| CAMSAP2 | protein_coding | | | | | | |
| TGFB1I1 | protein_coding | | | | | | |
| LINC01125 | protein_coding | | | | | | |
| KLF1 | protein_coding | | | | | | |
| STK32B | protein_coding | | | | | | |
| LTK | protein_coding | | | 1.21 | 0.00000018 | 1.39 | 0.0000015 |
| MADCAM1 | protein_coding | | | | | | |
| ZC3H12B | protein_coding | | | | | | |
| C1QC | protein_coding | | | | | | |
| WRNIP1 | protein_coding | | | | | | |
| CCDC34 | protein_coding | | | | | | |
| HAS1 | protein_coding | | | | | | |
| SH3BGRL2 | protein_coding | | | | | | |
| ORMDL2 | protein_coding | | | | | | |
| C17orf80 | protein_coding | | | | | | |
| PLCD3 | protein_coding | | | | | | |
| EMID1 | protein_coding | | | | | | |
| UBE2T | protein_coding | | | | | | |
| SLC22A23 | protein_coding | | | | | | |
| NUDT6 | protein_coding | | | | | | |
| GSPT2 | protein_coding | | | 0.79 | 0.016 | 1.27 | 0.0066 |
| EMILIN2 | protein_coding | | | | | | |
| CXCR1 | protein_coding | | | | | | |
| MRC1 | protein_coding | | | | | | |
| F2RL3 | protein_coding | | | | | | |
| ZBTB3 | protein_coding | | | | | | |
| LRRC61 | protein_coding | | | | | | |
| NUDT7 | protein_coding | | | | | −2.23 | 0.0028 |
| EYS | protein_coding | | | | | | |
| ZNF81 | protein_coding | | | | | | |
| ZNF778 | protein_coding | | | | | | |
| SLCO5A1 | protein_coding | | | | | | |
| SWT1 | protein_coding | | | | | | |
| RACGAP1 | protein_coding | | | | | | |
| MAPK11 | protein_coding | | | | | | |
| ZBTB47 | protein_coding | | | | | | |
| MYPOP | protein_coding | | | | | | |
| ZNRF3 | protein_coding | | | −0.84 | 0.00000081 | | |
| IPO4 | protein_coding | | | | | | |
| ITGB3BP | protein_coding | | | | | | |
| SMIM18 | protein_coding | | | | | | |
| CACNA1F | protein_coding | | | | | | |
| CEP112 | protein_coding | | | | | | |
| AP3B2 | protein_coding | | | | | | |
| CLEC4F | protein_coding | | | | | | |
| ITPRIPL1 | protein_coding | | | | | | |
| WDR5B | protein_coding | | | | | | |
| IQCC | protein_coding | | | | | | |
| DHDH | protein_coding | | | | | | |
| AASS | protein_coding | | | 0.87 | 0.011 | 1.13 | 0.0066 |
| CNIH2 | protein_coding | | | −2.18 | 0.017 | −1.89 | 0.025 |
| HCN2 | protein_coding | | | −1.89 | 0.049 | | |
| IL10RB | protein_coding | | | −1.66 | 0.024 | | |
| DMXL2 | protein_coding | | | −1.62 | 0.028 | | |
| PAQR4 | protein_coding | | | −1.56 | 0.0024 | −1.79 | 0.0014 |
| AC104581.1 | protein_coding | 1.27 | 0.000065 | | | 1.74 | 0.0019 |
| ACACA | protein_coding | | | 1.22 | 0.012 | 1.36 | 0.017 |
| REG4 | protein_coding | | | | | −0.62 | 0.022 |
| ADGRB2 | protein_coding | | | −1.49 | 0.043 | | |
| ACRBP | protein_coding | | | | | −0.6 | 0.0022 |
| ACSM3 | protein_coding | | | 1.23 | 0.0056 | | |
| ACTN1 | protein_coding | | | 1.36 | 0.012 | | |
| KDELR1 | protein_coding | | | −1.19 | 0.031 | | |
| PLOD3 | protein_coding | | | 1 | 0.0047 | 0.76 | 0.045 |
| RETREG3 | protein_coding | | | −1.06 | 0.012 | | |
| TACR2 | protein_coding | | | −1.04 | 9.8E−16 | | |
| TMEM203 | protein_coding | | | −0.99 | 0.0017 | | |
| VAMP4 | protein_coding | | | −0.96 | 0.009 | −0.98 | 0.018 |
| ADM5 | protein_coding | | | 1.2 | 0.000009 | 0.77 | 0.00058 |
| AFAP1L2 | protein_coding | | | 0.69 | 0.022 | | |
| AIF1 | protein_coding | | | 1.33 | 0.00068 | 1.08 | 0.012 |
| RNF5 | protein_coding | | | −0.91 | 0.019 | | |
| AK5 | protein_coding | | | 1.07 | 0.045 | | |
| AKAP3 | protein_coding | | | | | | |
| THAP4 | protein_coding | | | −0.88 | 0.0061 | | |
| ALDH3B1 | protein_coding | | | | | | |
| ALG13 | protein_coding | | | 1.6 | 0.0066 | | |
| ALOX5 | protein_coding | | | 1.3 | 0.022 | | |
| AMACR | protein_coding | | | −1.44 | 0.043 | | |
| AMER1 | protein_coding | | | −2.29 | 0.002 | | |
| ANKRD44 | protein_coding | | | 0.79 | 0.035 | | |
| TMEM179B | protein_coding | | | −0.84 | 0.0062 | | |
| APOBEC3A | protein_coding | | | 0.65 | 0.0000042 | 0.75 | 0.00045 |
| KDELR2 | protein_coding | | | −0.82 | 0.018 | | |
| KCNQ4 | protein_coding | | | −0.69 | 1.8E−26 | −0.61 | 8.1E−21 |
| ARHGAP33 | protein_coding | | | 1.74 | 0.000092 | 1.43 | 0.007 |
| ARMC9 | protein_coding | | | | | −0.87 | 0.043 |
| FPR1 | protein_coding | | | | | 0.85 | 0.000002 |
| G0S2 | protein_coding | | | | | 0.74 | 0.0000032 |
| ASXL2 | protein_coding | | | 1.65 | 0.00015 | | |
| ATIC | protein_coding | | | | | | |
| CALCRL | protein_coding | | | 0.64 | 5.5E−13 | 0.64 | 0.0000047 |
| B3GNT5 | protein_coding | | | 0.78 | 0.01 | | |
| B4GALNT1 | protein_coding | | | | | 0.62 | 0.0015 |
| BAAT | protein_coding | | | −0.58 | 5E−13 | | |
| BAIAP2L1 | protein_coding | | | −1.82 | 0.00012 | −1.11 | 0.0017 |
| BATF3 | protein_coding | | | 1.86 | 0.0056 | | |
| TNFSF13B | protein_coding | | | 1.24 | 0.00000023 | 1.16 | 0.000011 |
| BCL7A | protein_coding | | | 1.54 | 0.038 | | |
| STK36 | protein_coding | | | 2.76 | 0.00000024 | 2.47 | 0.000035 |
| BLK | protein_coding | | | 1.12 | 0.027 | | |
| BTBD19 | protein_coding | | | | | 1.44 | 0.019 |
| BTBD6 | protein_coding | | | 1.17 | 0.0016 | 1.74 | 0.00068 |
| BTBD9 | protein_coding | | | 1.19 | 0.0039 | | |
| BTK | protein_coding | | | | | 0.6 | 0.027 |
| ZDHHC14 | protein_coding | | | 1.57 | 0.00029 | 2.22 | 0.00013 |
| C16orf71 | protein_coding | | | −0.59 | 4.4E−10 | | |
| C16orf86 | protein_coding | | | 1.04 | 0.0063 | | |
| CTLA4 | protein_coding | | | 1.13 | 0.0046 | 1.95 | 0.00022 |
| C17orf98 | protein_coding | | | | | | |
| PCYOX1L | protein_coding | | | | | 0.84 | 0.046 |
| C2CD2 | protein_coding | | | 0.66 | 0.049 | 0.73 | 0.05 |
| C4orf19 | protein_coding | | | | | 1.29 | 0.0055 |
| C4orf33 | protein_coding | | | 0.82 | 0.011 | 0.87 | 0.037 |
| LGALS3BP | protein_coding | | | 1.13 | 0.00035 | 1.12 | 0.0023 |
| C8orf46 | protein_coding | | | 0.6 | 0.00082 | 0.66 | 0.0032 |
| C9orf40 | protein_coding | | | | | −1.74 | 0.0016 |
| EPHB3 | protein_coding | | | | | 0.62 | 0.00022 |
| TSSK4 | protein_coding | | | 1.12 | 0.0012 | 1.38 | 0.00026 |
| SEMA6B | protein_coding | | | | | 1.68 | 0.00026 |
| RGMB | protein_coding | | | 1.06 | 0.0000082 | 0.62 | 0.00078 |
| CAMSAP1 | protein_coding | | | 1.25 | 0.0068 | | |
| CAPN8 | protein_coding | | | −1.06 | 2E−32 | −0.58 | 2.1E−18 |
| CARD6 | protein_coding | | | −1.56 | 0.0012 | | |
| CD300C | protein_coding | | | 1.15 | 0.011 | 1.39 | 0.001 |
| CASP6 | protein_coding | | | 0.7 | 0.048 | | |
| CBX8 | protein_coding | −0.87 | 0.0048 | | | | |
| SLC19A1 | protein_coding | | | 1.12 | 0.00036 | 1.42 | 0.0011 |
| GPM6B | protein_coding | 0.73 | 0.013 | | | 1.47 | 0.0011 |
| CCDC184 | protein_coding | | | | | | |
| CCM2 | protein_coding | | | −0.92 | 0.021 | | |
| CCNB1 | protein_coding | | | | | | |
| LHFPL2 | protein_coding | | | | | 0.9 | 0.0014 |
| FCN1 | protein_coding | | | 1.2 | 0.00036 | 1.38 | 0.0015 |
| SV2A | protein_coding | | | 1.72 | 0.000013 | 1.19 | 0.0016 |
| MCEMP1 | protein_coding | | | 1.05 | 0.00091 | 1.59 | 0.0019 |
| IL10 | protein_coding | | | | | 1.15 | 0.0036 |
| KCNH3 | protein_coding | | | 0.77 | 0.0034 | 1.01 | 0.0022 |
| TTYH3 | protein_coding | | | 1.86 | 0.00011 | 1.85 | 0.003 |
| TMEM170B | protein_coding | | | 1.55 | 0.00033 | 1.55 | 0.0032 |
| FAM98B | protein_coding | | | 2.23 | 0.0000048 | 1.75 | 0.0033 |
| NTSR1 | protein_coding | | | 0.72 | 3.9E−11 | 0.71 | 0.0038 |
| CDCA7 | protein_coding | | | 1 | 0.0067 | | |
| SLC24A4 | protein_coding | | | | | 0.81 | 0.004 |
| SIGLEC14 | protein_coding | | | | | 0.65 | 0.0043 |
| CEBPD | protein_coding | | | 1.28 | 0.0032 | 1.58 | 0.0027 |
| PGLYRP2 | protein_coding | | | | | 0.81 | 0.0055 |
| CEP83 | protein_coding | | | 1.12 | 0.038 | | |
| OR56B1 | protein_coding | | | 0.59 | 0.0000021 | 0.6 | 0.0081 |
| CFAP70 | protein_coding | | | | | 1.92 | 0.00021 |
| CHAMP1 | protein_coding | | | 1.34 | 0.025 | | |
| CHD6 | protein_coding | 0.63 | 0.049 | | | 0.84 | 0.013 |
| FNDC10 | protein_coding | | | | | 1.23 | 0.0089 |
| CACFD1 | protein_coding | | | 0.94 | 0.00046 | 0.61 | 0.009 |
| FITM2 | protein_coding | | | 1.28 | 0.0072 | 1.54 | 0.0094 |
| ASIC3 | protein_coding | | | | | 1.03 | 0.013 |
| AC007040.2 | protein_coding | | | 0.66 | 3.8E−10 | 0.65 | 0.018 |
| LRRC3 | protein_coding | | | | | 0.73 | 0.018 |
| CMBL | protein_coding | | | −0.92 | 9.7E−11 | | |
| CNFN | protein_coding | | | −0.98 | 0.0045 | | |
| FANCA | protein_coding | | | 1.03 | 0.0056 | 1.05 | 0.02 |
| OR1L8 | protein_coding | | | 0.64 | 0.000000022 | 0.63 | 0.021 |
| ASTL | protein_coding | | | | | 1.38 | 0.0091 |
| S100A8 | protein_coding | | | 1.5 | 0.0009 | 1.42 | 0.0036 |
| MTRNR2L3 | protein_coding | | | 1.23 | 0.0086 | 1.45 | 0.0073 |
| S100A9 | protein_coding | | | 1.51 | 0.00057 | 1.59 | 0.0011 |
| COQ9 | protein_coding | | | 0.8 | 0.0000001 | 0.75 | 0.0000035 |
| IL17C | protein_coding | | | 1.54 | 0.049 | 1.86 | 0.031 |
| CRAMP1 | protein_coding | | | | | | |
| CNTNAP1 | protein_coding | | | | | 1.75 | 0.021 |
| MEGF8 | protein_coding | | | | | 1.67 | 0.022 |
| PRSS22 | protein_coding | | | | | 2.02 | 0.007 |
| CRYBB2 | protein_coding | | | | | | |
| MFSD6L | protein_coding | | | 0.59 | 0.0000028 | 0.58 | 0.028 |
| CFP | protein_coding | | | 2.07 | 0.00000012 | 2.03 | 0.000014 |
| SLC35G5 | protein_coding | | | 0.59 | 0.000000099 | 0.58 | 0.029 |
| LYZ | protein_coding | | | 1.68 | 0.000079 | 2.04 | 0.00011 |
| MMP17 | protein_coding | | | −0.58 | 0.00012 | | |
| LAPTM4B | protein_coding | | | 1.04 | 0.0048 | 0.76 | 0.031 |
| PRRG4 | protein_coding | | | | | 1.15 | 0.031 |
| CYGB | protein_coding | | | | | | |
| SLC1A2 | protein_coding | | | 1.35 | 0.016 | 1.38 | 0.032 |
| MT-ND1 | protein_coding | | | | | 0.64 | 0.032 |
| QPCT | protein_coding | | | 0.62 | 0.0029 | | |
| SEMA3B | protein_coding | | | 0.63 | 0.000000017 | | |
| PLXNA4 | protein_coding | | | 1.45 | 0.002 | 0.89 | 0.035 |
| CYB561D1 | protein_coding | | | 1.39 | 0.00051 | 0.79 | 0.038 |
| DDN | protein_coding | | | | | 1.23 | 0.0002 |
| CCR8 | protein_coding | | | | | 0.86 | 0.038 |
| ATP6V0A1 | protein_coding | | | | | 1.2 | 0.048 |
| DHRS12 | protein_coding | | | 1.13 | 0.045 | | |
| C8G | protein_coding | | | 0.71 | 0.018 | | |
| TMEM243 | protein_coding | | | | | 0.59 | 0.049 |
| DNA2 | protein_coding | | | 1.52 | 0.017 | 1.33 | 0.027 |
| PGAP3 | protein_coding | | | 1.41 | 0.0056 | 1.23 | 0.05 |
| DNMT3B | protein_coding | | | | | | |
| DOCK10 | protein_coding | | | 1.15 | 0.0059 | | |
| FAR2 | protein_coding | | | | | 1.3 | 0.05 |
| MARCO | protein_coding | | | 0.59 | 3.9E−17 | | |
| DTX1 | protein_coding | | | 0.8 | 0.00047 | | |
| CDH5 | protein_coding | | | 0.58 | 4E−11 | | |
| DUSP28 | protein_coding | | | 1.16 | 0.026 | | |
| DUSP6 | protein_coding | | | 1.46 | 0.022 | | |
| DZANK1 | protein_coding | | | 1.13 | 0.049 | | |
| CLCN1 | protein_coding | | | 0.62 | 5.8E−11 | | |
| EFCAB12 | protein_coding | | | 0.88 | 0.033 | 2.2 | 0.00041 |
| EFHC2 | protein_coding | | | | | | |
| EIF1AX | protein_coding | | | 1.12 | 0.0092 | | |
| DPP4 | protein_coding | | | 1.34 | 0.0000056 | | |
| COL1A1 | protein_coding | | | 0.98 | 0.017 | | |
| EML6 | protein_coding | | | | | 0.77 | 0.008 |
| ENO4 | protein_coding | | | 0.6 | 2.8E−14 | 0.61 | 0.00056 |
| ENOX2 | protein_coding | | | 1.35 | 0.0045 | | |
| EPS8L1 | protein_coding | | | −2.13 | 0.00099 | −2.1 | 0.0091 |
| EXOC6B | protein_coding | | | 1.46 | 0.0062 | 1.42 | 0.043 |
| GLTID1 | protein_coding | | | 1.01 | 0.002 | | |
| ATP8B3 | protein_coding | | | 1.77 | 0.00011 | | |
| B3GALT2 | protein_coding | | | 0.9 | 0.00056 | | |
| CES4A | protein_coding | | | 1.06 | 0.0000074 | | |
| FAM109B | protein_coding | | | 1.23 | 0.00000021 | 1.74 | 0.00000018 |
| FAM161B | protein_coding | −0.68 | 0.0068 | | | −1.21 | 0.027 |
| GPR75 | protein_coding | | | 1.13 | 0.0006 | | |
| DRD3 | protein_coding | | | 1.39 | 0.00066 | | |
| FAM212B | protein_coding | | | 1.56 | 0.023 | 2.43 | 0.00058 |
| KIAA0319L | protein_coding | | | 1.62 | 0.00078 | | |
| FAM216A | protein_coding | | | 1.06 | 0.014 | | |
| FAM227B | protein_coding | | | 1.16 | 0.011 | 1.44 | 0.017 |
| RMND1 | protein_coding | | | 1.51 | 0.00085 | | |
| SLC38A7 | protein_coding | | | 1.72 | 0.000064 | 2.12 | 0.00027 |
| FANCL | protein_coding | | | 1.22 | 0.048 | | |
| MS4A6A | protein_coding | | | 0.6 | 0.0011 | | |
| COL9A2 | protein_coding | | | 1.2 | 0.0093 | | |
| FBP1 | protein_coding | | | 1.42 | 0.003 | 1.58 | 0.024 |
| FBXO2 | protein_coding | | | 0.99 | 0.023 | | |
| CHIC1 | protein_coding | | | 1.52 | 0.0012 | | |
| FDXR | protein_coding | | | 0.82 | 0.026 | | |
| MBOAT2 | protein_coding | | | 1.03 | 0.002 | | |
| LYSMD4 | protein_coding | | | 1.85 | 0.0028 | | |
| TPCN1 | protein_coding | | | 1.52 | 0.0044 | | |
| FTCDNL1 | protein_coding | | | 1.24 | 0.000022 | | |
| TBXAS1 | protein_coding | | | 1.78 | 0.0048 | | |
| TVP23C | protein_coding | | | 1.56 | 0.0052 | | |
| FOSL1 | protein_coding | | | 1.25 | 0.0099 | | |
| FOXRED2 | protein_coding | | | 1.2 | 0.045 | | |
| SPON1 | protein_coding | | | 1.25 | 0.033 | | |
| TMEM238 | protein_coding | | | 2.02 | 0.0059 | | |
| CDHR1 | protein_coding | | | 1.34 | 0.0065 | | |
| IL12A | protein_coding | | | 1.28 | 0.0066 | | |
| KCNQ5 | protein_coding | | | 1.66 | 0.007 | | |
| FXYD2 | protein_coding | | | 1.29 | 0.012 | | |
| GBGT1 | protein_coding | | | | | | |
| GCA | protein_coding | | | 1.07 | 0.04 | | |
| GCAT | protein_coding | | | | | | |
| SLC25A17 | protein_coding | | | | | −1.93 | 0.0086 |
| SLC4A8 | protein_coding | | | 1.11 | 0.013 | | |
| GFRA2 | protein_coding | | | 0.66 | 0.00000012 | 0.64 | 0.018 |
| GGACT | protein_coding | | | 1.05 | 0.0065 | | |
| GGCT | protein_coding | | | | | | |
| GINS3 | protein_coding | | | | | | |
| GIPC3 | protein_coding | | | −1.3 | 0.04 | | |
| GIPR | protein_coding | | | | | −1.68 | 0.033 |
| F5 | protein_coding | | | 1.37 | 0.017 | | |
| GNG12 | protein_coding | | | 0.64 | 0.0000041 | 0.62 | 0.021 |
| GPATCH2L | protein_coding | | | 1.39 | 0.0047 | 1.6 | 0.0037 |
| PODXL | protein_coding | −0.73 | 0.00082 | | | −1.4 | 0.021 |
| SLC25A42 | protein_coding | | | | | −1.4 | 0.033 |
| SEMA6C | protein_coding | −0.63 | 0.0078 | | | −1.11 | 0.047 |
| CD320 | protein_coding | | | | | −1.09 | 0.038 |
| RDM1 | protein_coding | | | | | −0.98 | 0.048 |
| GPRIN1 | protein_coding | | | 0.74 | 0.00024 | | |
| GRASP | protein_coding | | | | | 2.09 | 0.0091 |
| NOTCH4 | protein_coding | | | 1.72 | 0.015 | | |
| GSTM2 | protein_coding | | | 1.37 | 0.0011 | | |
| MAOA | protein_coding | | | | | −0.63 | 2.2E−16 |
| HADH | protein_coding | | | 0.67 | 0.000035 | 0.81 | 0.00014 |
| HARBI1 | protein_coding | | | 1.27 | 0.018 | | |
| C14orf132 | protein_coding | | | | | −0.61 | 0.031 |
| HCFC1 | protein_coding | | | | | | |
| SYT6 | protein_coding | | | | | −0.58 | 0.0006 |
| HDAC9 | protein_coding | | | | | | |
| TMPRSS2 | protein_coding | | | | | 0.58 | 0.0051 |
| MEMO1 | protein_coding | | | 1.67 | 0.016 | | |
| HIST1H2AE | protein_coding | | | 1.31 | 0.000042 | 1.99 | 0.000033 |
| HIST1H2BF | protein_coding | | | 1 | 0.0015 | | |
| HIST1H2BM | protein_coding | | | 1.54 | 0.0027 | 1.58 | 0.0071 |
| HIST1H3E | protein_coding | | | | | 1.05 | 0.0062 |
| HIST1H3I | protein_coding | | | 0.63 | 0.013 | 1.11 | 0.0055 |
| HIST1H4E | protein_coding | | | 1.29 | 0.02 | | |
| HIST1H4I | protein_coding | | | −1.58 | 0.037 | | |
| HLF | protein_coding | | | −0.9 | 8.5E−20 | | |
| BIK | protein_coding | | | 1.3 | 0.016 | | |
| HOMER1 | protein_coding | | | 1.18 | 0.032 | | |
| HOOK1 | protein_coding | | | | | 1.13 | 0.049 |
| HOXA1 | protein_coding | | | 0.86 | 0.026 | 1.05 | 0.0014 |
| HSD17B6 | protein_coding | | | 1.19 | 0.0000035 | 1.31 | 0.0000034 |
| HSD17B7 | protein_coding | | | 1.09 | 0.018 | | |
| HSPA13 | protein_coding | | | 1.47 | 0.0025 | 1.56 | 0.0041 |
| IFI44L | protein_coding | | | 1.75 | 0.0024 | 1.69 | 0.0087 |
| IFT140 | protein_coding | | | 1.8 | 0.000073 | | |
| IFT172 | protein_coding | | | | | | |
| SDR42E2 | protein_coding | | | 1.33 | 0.019 | | |
| SEMA3G | protein_coding | | | | | | |
| CCDC136 | protein_coding | | | 1.84 | 0.02 | | |
| APOO | protein_coding | | | 0.83 | 0.023 | | |
| IMPACT | protein_coding | | | 1.42 | 0.0016 | | |
| ISPD | protein_coding | | | 1.15 | 0.032 | | |
| ITPKC | protein_coding | | | | | 0.84 | 0.043 |
| SEMA6A | protein_coding | | | 1.64 | 0.025 | | |
| JSRP1 | protein_coding | | | 2.07 | 0.0000084 | | |
| JUP | protein_coding | | | | | 1.24 | 0.0059 |
| KBTBD8 | protein_coding | | | 0.93 | 0.036 | | |
| LTC4S | protein_coding | | | 0.6 | 0.025 | | |
| PTPRS | protein_coding | | | 2.18 | 0.028 | | |
| PLPP1 | protein_coding | | | 1.41 | 0.032 | | |
| PLXDC2 | protein_coding | | | 0.76 | 0.032 | | |
| CLN6 | protein_coding | | | 0.74 | 0.032 | | |
| MT-ND3 | protein_coding | | | 0.84 | 0.033 | | |
| NDFIP2 | protein_coding | | | 0.67 | 0.035 | | |
| METTL7A | protein_coding | | | 0.6 | 0.035 | | |
| KDM8 | protein_coding | | | 0.93 | 0.043 | | |
| CCDC163 | protein_coding | | | 1.33 | 0.036 | | |
| KIAA0825 | protein_coding | | | 1.29 | 0.0058 | | |
| KIF1BP | protein_coding | | | 0.97 | 0.00000069 | | |
| KIF24 | protein_coding | | | −0.65 | 3.6E−11 | | |
| KIF5C | protein_coding | | | 1.5 | 0.0044 | 1.48 | 0.025 |
| NEMP1 | protein_coding | | | 0.82 | 0.036 | | |
| KMT2D | protein_coding | | | | | 1.69 | 0.016 |
| L3HYPDH | protein_coding | | | 1.33 | 0.0053 | | |
| CXADR | protein_coding | | | 1.19 | 0.039 | | |
| LANCL3 | protein_coding | | | | | 1.18 | 0.032 |
| ANO6 | protein_coding | | | 0.74 | 0.04 | | |
| LENG9 | protein_coding | | | −1.22 | 1E−31 | | |
| CYTL1 | protein_coding | | | | | | |
| LGMN | protein_coding | | | 1.75 | 0.000082 | | |
| SLC37A4 | protein_coding | | | 0.86 | 0.045 | | |
| NPHP4 | protein_coding | | | | | | |
| PTGIR | protein_coding | | | | | | |
| LONRF3 | protein_coding | | | 1.82 | 0.033 | | |
| ZACN | protein_coding | | | | | | |
| LRRC75B | protein_coding | | | 0.76 | 0.0034 | 0.82 | 0.025 |
| RPAP1 | protein_coding | | | | | | |
| LYPD2 | protein_coding | | | | | 0.59 | 0.0013 |
| INSL4 | protein_coding | | | | | | |
| MAFK | protein_coding | | | | | 1.9 | 0.028 |
| MAML3 | protein_coding | | | 1.34 | 0.037 | 1.98 | 0.0067 |
| MAMLD1 | protein_coding | | | 0.79 | 0.000032 | | |
| MAP2K6 | protein_coding | | | 1.31 | 0.023 | | |
| MAP3K21 | protein_coding | | | 1.4 | 0.0006 | 1.71 | 0.00034 |
| MAP4K4 | protein_coding | | | 1.63 | 0.00037 | 1.62 | 0.0027 |
| MAPK15 | protein_coding | | | 0.6 | 0.000000098 | 0.59 | 0.028 |
| MAPK8IP1 | protein_coding | | | 1.36 | 0.00091 | 1.7 | 0.002 |
| PRUNE2 | protein_coding | | | | | | |
| MARS2 | protein_coding | | | 0.83 | 0.044 | | |
| TREML1 | protein_coding | | | | | | |
| CCR3 | protein_coding | | | | | | |
| VSIG4 | protein_coding | | | | | | |
| MSLN | protein_coding | | | | | | |
| TUBA8 | protein_coding | | | | | | |
| DUOXA1 | protein_coding | | | | | | |
| MGLL | protein_coding | | | 0.58 | 0.0024 | 1.32 | 0.0003 |
| FXYD6 | protein_coding | | | | | | |
| MKRN3 | protein_coding | | | 0.86 | 0.048 | | |
| MORN4 | protein_coding | | | | | 0.77 | 0.0065 |
| MROH8 | protein_coding | | | 1.41 | 0.0000058 | 1.51 | 0.000046 |
| MRPL34 | protein_coding | | | −0.76 | 0.021 | | |
| MSRB3 | protein_coding | | | 0.58 | 0.000000049 | 0.59 | 0.0049 |
| MT2A | protein_coding | | | 1 | 0.012 | 0.98 | 0.035 |
| MTSS1L | protein_coding | | | | | 0.71 | 0.017 |
| MTX2 | protein_coding | | | 0.96 | 0.021 | | |
| MYCBP2 | protein_coding | | | 0.95 | 0.042 | | |
| MYLPF | protein_coding | | | | | 1.77 | 0.026 |
| MYOM2 | protein_coding | | | −2.72 | 0.029 | | |
| NAF1 | protein_coding | | | 1.56 | 0.0043 | | |
| NAPA | protein_coding | | | | | | |
| NEK1 | protein_coding | | | 1.12 | 0.049 | | |
| NHLH2 | protein_coding | | | 0.73 | 0.000000054 | | |
| NPM2 | protein_coding | | | | | | |
| NSUN4 | protein_coding | | | 1.22 | 0.042 | | |
| NUBP2 | protein_coding | | | −0.79 | 0.046 | | |
| NUDT18 | protein_coding | | | 1.09 | 0.0039 | 1.04 | 0.016 |
| OGG1 | protein_coding | | | 1.39 | 0.0000062 | 1.35 | 0.00024 |
| JOLIG1 | protein_coding | | | | | 0.62 | 0.0027 |
| OPHN1 | protein_coding | | | 0.66 | 0.000000018 | | |
| OTUD7B | protein_coding | | | | | | |
| OVGP1 | protein_coding | | | 0.64 | 0.032 | 0.85 | 0.032 |
| PAFAH1B3 | protein_coding | | | 1.24 | 0.039 | | |
| PAH | protein_coding | | | 0.6 | 0.0000015 | 0.59 | 0.026 |
| PARG | protein_coding | | | | | | |
| PARS2 | protein_coding | | | −1.75 | 2.7E−09 | −1.38 | 0.00000011 |
| PCCA | protein_coding | | | | | 1.49 | 0.0013 |
| PELP1 | protein_coding | | | | | −1.3 | 0.022 |
| PGM2 | protein_coding | | | | | 1.37 | 0.006 |
| PHLPP1 | protein_coding | | | | | 1.23 | 0.018 |
| PIFO | protein_coding | | | −2.18 | 0.0000038 | −1.41 | 0.000068 |
| PLEKHB1 | protein_coding | | | 0.87 | 0.011 | | |
| PLK1 | protein_coding | | | | | | |
| PLK4 | protein_coding | | | 0.8 | 0.00027 | | |
| PMS2 | protein_coding | | | | | 1.38 | 0.04 |
| PNKP | protein_coding | | | | | | |
| POLD1 | protein_coding | | | 1.79 | 3.5E−10 | 1.24 | 0.0000048 |
| PPL | protein_coding | | | 2.3 | 0.00000013 | 2 | 0.00006 |
| PPM1H | protein_coding | | | 0.64 | 0.00000021 | 0.63 | 0.02 |
| PPP4R1 | protein_coding | | | | | −1.33 | 0.033 |
| PRC1 | protein_coding | | | | | | |
| PRDM13 | protein_coding | | | | | 0.63 | 0.013 |
| PRKD2 | protein_coding | | | −0.78 | 0.026 | −0.82 | 0.033 |
| PRR34 | protein_coding | | | | | 0.68 | 0.00053 |
| GREM2 | protein_coding | | | | | | |
| PSMA8 | protein_coding | | | 0.67 | 0.0033 | 0.91 | 0.0028 |
| PSRC1 | protein_coding | | | | | 0.9 | 0.023 |
| PUS3 | protein_coding | | | | | 1.39 | 0.008 |
| PYCR3 | protein_coding | | | 0.82 | 0.035 | | |
| RAB26 | protein_coding | | | | | | |
| RAD54L | protein_coding | | | | | 1.37 | 0.000000015 |
| RALGPS2 | protein_coding | | | 0.64 | 0.0052 | 1.47 | 0.00089 |
| RASGRF2 | protein_coding | | | 1.31 | 0.0024 | 1.41 | 0.0042 |
| RASL11A | protein_coding | | | 0.77 | 0.039 | | |
| RBFA | protein_coding | | | 1.19 | 0.0097 | | |
| RBMS2 | protein_coding | | | 1.13 | 0.03 | | |
| REXO5 | protein_coding | | | | | 0.81 | 0.002 |
| RIMS3 | protein_coding | | | −2 | 0.023 | | |
| RNF141 | protein_coding | | | 1.21 | 0.0066 | | |
| RPL10A | protein_coding | | | | | 0.62 | 0.029 |
| RPL34 | protein_coding | | | 0.76 | 0.026 | 0.83 | 0.025 |
| RPL37 | protein_coding | | | 0.78 | 0.042 | | |
| RPL6 | protein_coding | | | 0.69 | 0.02 | 0.71 | 0.036 |
| RPP30 | protein_coding | | | 1.04 | 0.04 | | |
| RPS21 | protein_coding | | | | | 0.81 | 0.048 |
| RPS24 | protein_coding | | | 0.86 | 0.016 | | |
| RSPH1 | protein_coding | | | 0.65 | 2.5E−09 | 0.65 | 0.014 |
| RSPH9 | protein_coding | | | | | | |
| RTN4IP1 | protein_coding | | | 1.51 | 0.032 | | |
| SAFB | protein_coding | | | | | | |
| SASH3 | protein_coding | | | −0.77 | 0.026 | | |
| SCML1 | protein_coding | | | | | 2.24 | 0.02 |
| SCML4 | protein_coding | | | 1.13 | 0.016 | | |
| SCRIB | protein_coding | | | 1.27 | 0.019 | | |
| SCYL1 | protein_coding | | | −0.8 | 0.041 | | |
| SGK3 | protein_coding | | | 1.24 | 0.046 | | |
| SH2B2 | protein_coding | | | | | 0.79 | 0.00095 |
| SH2D7 | protein_coding | | | | | | |
| SLC25A30 | protein_coding | | | | | | |
| SMARCD3 | protein_coding | | | 1.38 | 0.044 | | |
| SMC1A | protein_coding | | | 1.13 | 0.028 | | |
| SOBP | protein_coding | −0.87 | 0.018 | | | −1.46 | 0.0049 |
| SOCS6 | protein_coding | | | 0.72 | 0.0073 | | |
| SORD | protein_coding | | | 1.09 | 0.016 | | |
| SOWAHD | protein_coding | | | | | | |
| SOX12 | protein_coding | | | | | | |
| SPAG1 | protein_coding | | | 2.04 | 0.00016 | | |
| SPRN | protein_coding | | | 2.05 | 0.000000011 | 1.32 | 0.0033 |
| CYP2U1 | protein_coding | | | | | | |
| MPIG6B | protein_coding | | | | | | |
| STARD5 | protein_coding | | | 1.1 | 0.005 | | |
| STK19 | protein_coding | | | 1.23 | 0.021 | | |
| STPG1 | protein_coding | | | | | 1.12 | 0.000062 |
| SUV39H2 | protein_coding | | | 1.06 | 0.041 | | |
| SYK | protein_coding | | | | | | |
| SZT2 | protein_coding | | | 2.1 | 0.00000015 | 2.11 | 0.000007 |
| TAF1A | protein_coding | | | 1.77 | 0.0081 | 2.27 | 0.0021 |
| TBC1D4 | protein_coding | | | 1.45 | 0.0056 | | |
| TBCK | protein_coding | | | 1.73 | 0.0046 | | |
| TBX3 | protein_coding | | | 0.66 | 0.000000029 | 0.64 | 0.019 |
| TEP1 | protein_coding | | | 1.88 | 0.0000012 | 1.56 | 0.00045 |
| TET3 | protein_coding | | | 1.25 | 0.015 | | |
| TFAP2E | protein_coding | | | −2.57 | 0.000086 | −2.25 | 0.00034 |
| TIAM1 | protein_coding | | | 1.31 | 0.00016 | 0.8 | 0.0074 |
| TMEM256-PLSCR3 | protein_coding | | | | | 0.62 | 0.016 |
| TRIM58 | protein_coding | | | 0.65 | 0.00075 | | |
| TSR2 | protein_coding | | | | | −0.64 | 0.017 |
| TTLL5 | protein_coding | | | 1.42 | 0.0077 | | |
| TUBB | protein_coding | | | −0.79 | 0.015 | | |
| UCHL3 | protein_coding | | | | | | |
| UPF3A | protein_coding | | | | | −0.68 | 0.013 |
| USP40 | protein_coding | | | 1.83 | 0.00049 | 1.64 | 0.0066 |
| VPS50 | protein_coding | | | 1.6 | 0.027 | | |
| WASF1 | protein_coding | | | 1.66 | 0.000046 | 1 | 0.033 |
| WDR44 | protein_coding | | | | | 1.34 | 0.05 |
| WDR86 | protein_coding | | | | | 1.65 | 0.022 |
| ZBTB10 | protein_coding | | | | | | |
| ZKSCAN4 | protein_coding | | | −1.64 | 0.022 | −2.34 | 0.00096 |
| ZNF138 | protein_coding | | | 1.4 | 0.026 | | |
| ZNF20 | protein_coding | | | −1.18 | 0.0048 | −0.96 | 0.0017 |
| ZNF23 | protein_coding | | | 1.45 | 0.0062 | | |
| ZNF257 | protein_coding | | | 1.33 | 0.022 | 1.44 | 0.037 |
| ZNF280B | protein_coding | | | 1.06 | 0.011 | | |
| ZNF304 | protein_coding | | | 0.69 | 0.0071 | | |
| ZNF318 | protein_coding | | | | | | |
| ZNF324B | protein_coding | | | | | 1.75 | 0.014 |
| ZNF460 | protein_coding | | | 1.24 | 0.0062 | 0.95 | 0.026 |
| ZNF544 | protein_coding | | | 0.81 | 0.0037 | 1.37 | 0.0011 |
| ZNF599 | protein_coding | | | 0.87 | 0.03 | | |
| ZNF630 | protein_coding | 0.63 | 0.0027 | 1.5 | 0.0033 | 2.11 | 0.000077 |
| ZNF646 | protein_coding | | | | | | |
| ZNF726 | protein_coding | | | | | | |
| ZNF736 | protein_coding | | | 2.05 | 0.0055 | 2.05 | 0.019 |
| ZNF841 | protein_coding | | | 1.58 | 0.0039 | | |
| ZNF93 | protein_coding | | | | | | |
| ZNHIT1 | protein_coding | | | −0.64 | 0.027 | | |
| ZSWIM6 | protein_coding | | | 1.3 | 0.024 | 1.32 | 0.037 |
| | | 9 | | 333 | | 227 | |

TABLE 4
Surface expressing and secretory targets in different comparisons:

| | PBMC | | | CD4 memory T cells | | | CD8 memory T cells | | |
|---|---|---|---|---|---|---|---|---|---|
| | PD | PD_R | PD_R | PD | PD_R | PD_R | PD | PD_R | PD_R |
| | vs | vs | vs | vs | vs | vs | vs | vs | vs |
| Comparison | HC | PD_NR | HC_NR | HC | PD_NR | HC_NR | HC | PD_NR | HC_NR |
| DE genes | 26 | 132 | 101 | 16 | 503 | 260 | 14 | 494 | 356 |
| Protein | 18 | 90 | 65 | 11 | 304 | 172 | 9 | 333 | 227 |
| coding | | | | | | | | | |
| SE genes | 9 | 39 | 25 | 4 | 133 | 76 | 3 | 140 | 100 |
| Down | DCHS1 | POPDC2 | P2RY6 | ZACN | CALHM2 | SYK | SEMA6C | KDELR2 | PRKD2 |
| regulated | TMTC1 | GPR171 | EPHX3 | GREM2 | MAMDC4 | CDA | PODXL | DMXL2 | ACRBP |
| Genes | WDR5B | AATK | NRP2 | CYP2U1 | HS6ST1 | LAT2 | | KCNQ4 | KCNQ4 |
| | CACNA1F | XPNPEP2 | ACE | | PEX26 | KCNH4 | | CNIH2 | CNIH2 |
| | | P2RY6 | SLC22A16 | | CDC42EP2 | DHCR24 | | IL10RB | CD320 |
| | | NAPSA | LRRC3 | | CD36 | CCR3 | | TMEM203 | GIPR |
| | | SLC45A4 | PRSS27 | | CD8A | NPHP4 | | BAIAP2L1 | SLC25A42 |
| | | PRPF40B | HFE | | MMP25 | F2RL2 | | MMP17 | UPF3A |
| | | FAM173B | FFAR1 | | CISH | COL16A1 | | TACR2 | MAOA |
| | | SLC22A16 | PRPF40B | | TNFAIP2 | CD300LB | | TMEM179B | BAIAP2L1 |
| | | DKK3 | CCR5 | | FGR | MSLN | | VAMP4 | SLC25A17 |
| | | NUDT6 | FUT2 | | ACAN | AP2A1 | | AMACR | PAQR4 |
| | | BIK | SDCBP2 | | ABCC3 | RAB26 | | RNF5 | SYT6 |
| | | C1QC | TMTC1 | | RAB6B | IGFBP6 | | HIST1H4I | C14orf132 |
| | | PLCD3 | DCHS1 | | TMEM201 | RNF152 | | EPS8L1 | PELP1 |
| | | MADCAM1 | CTRC | | IGFBP6 | ACAN | | CDC42EP2 | VAMP4 |
| | | HAS1 | COL4A2 | | CX3CR1 | CPB2 | | HCN2 | REG4 |
| | | HFE | | | F2RL2 | SLC15A2 | | ZNRF3 | SEMA6C |
| | | ORMDL2 | | | AP2A1 | CYTL1 | | PRKD2 | EPS8L1 |
| | | EMID1 | | | FBLN2 | ZACN | | PAQR4 | PODXL |
| | | C17orf80 | | | DHCR24 | LRRK2 | | KDELR1 | |
| | | STK32B | | | KCNN3 | TREML1 | | | |
| | | SLC22A23 | | | ITGB4 | WIPI1 | | | |
| | | CCR5 | | | SPINK4 | SEMA3G | | | |
| | | CXCR1 | | | MFSD8 | CD36 | | | |
| | | CLIP3 | | | ST6GALN | SLC7A8 | | | |
| | | LRRC3 | | | AC6 | VSIG4 | | | |
| | | F2RL3 | | | TBC1D8 | ABCC3 | | | |
| | | SLCO5A1 | | | FCGBP | IL22 | | | |
| | | HIGD1A | | | CSF3R | TOM1L1 | | | |
| | | MRC1 | | | XPNPEP2 | FXYD6 | | | |
| | | F2RL3 | | | SIGLEC7 | FFAR3 | | | |
| | | | | | SRC | GBGT1 | | | |
| | | | | | WIPI1 | STRC | | | |
| | | | | | LAT2 | SLC35F3 | | | |
| | | | | | PSKH1 | SCARA3 | | | |
| | | | | | RAB3D | MCOLN3 | | | |
| | | | | | CDA | LAMP3 | | | |
| | | | | | SELPLG | ZNF532 | | | |
| | | | | | COL16A1 | TRPM2 | | | |
| | | | | | TOM1L1 | MTUS1 | | | |
| | | | | | HHLA2 | PDIA5 | | | |
| | | | | | KCNH4 | AIG1 | | | |
| | | | | | RAB1B | INVS | | | |
| | | | | | RNF152 | TMEM97 | | | |
| | | | | | CPB2 | H6PD | | | |
| | | | | | CD300LB | FAM19A2 | | | |
| | | | | | SGCA | CYP2S1 | | | |
| | | | | | FFAR3 | LEAP2 | | | |
| | | | | | PBXIP1 | CASK | | | |
| | | | | | LRFN2 | SARNP | | | |
| | | | | | SLC7A8 | PTGIR | | | |
| | | | | | TAP1 | CSF3R | | | |
| | | | | | CHRNA10 | SLC25A30 | | | |
| | | | | | RET | RAB6B | | | |
| | | | | | EXOC3L2 | TMEM201 | | | |
| | | | | | IL22 | C2CD2 | | | |
| | | | | | ST3GAL6 | CHRNA10 | | | |
| | | | | | SLC15A2 | NAPA | | | |
| | | | | | GPR153 | | | | |
| | | | | | MS4A14 | | | | |
| | | | | | KIRREL2 | | | | |
| | | | | | LRRK2 | | | | |
| | | | | | SLC30A8 | | | | |
| | | | | | MPO | | | | |
| | | | | | OLFML2B | | | | |
| | | | | | LYPD4 | | | | |
| | | | | | IGDCC4 | | | | |
| | | | | | GML | | | | |
| | | | | | FGFBP1 | | | | |
| Upregulated | CLEC4F | TPR | PCBP2 | SOX12 | GYPE | AQP9 | GPM6B | MSRB3 | TMPRSS2 |
| genes | AP3B2 | EMILIN2 | CERK | | RASD1 | ECE2 | | LTC4S | GFRA2 |
| | ITPRIPL1 | ZNRF3 | LPAR1 | | CD180 | INSL4 | | METTL7A | MFSD6L |
| | EIF1AY | SPOCD1 | EIF1AY | | B3GNT8 | TOMM5 | | OVGP1 | LRRK2 |
| | CYP2F1 | CTDSPL | PEX3 | | STRC | SERPINH1 | | MAPK15 | SRC |
| | | EYS | PDPR | | SLC7A10 | HIST1H4H | | SLC19A1 | RGMB |
| | | GCNT7 | CRIM1 | | TOMM5 | GDF11 | | MTX2 | B4GALNT1 |
| | | | CYP2F1 | | RNASEL | ABCD2 | | COL1A1 | LRRC3 |
| | | | | | ADCK1 | CRB3 | | MBOAT2 | OVGP1 |
| | | | | | FUT11 | CELSR2 | | SORD | CCR1 |
| | | | | | INVS | MPHOSPH9 | | HSD17B7 | SLC19A1 |
| | | | | | CSF2RB | TNFRSF11A | | SLC4A8 | S100A8 |
| | | | | | EPHX1 | GALNT1 | | CDH5 | G0S2 |
| | | | | | HIST1H4H | APOL1 | | GNG12 | GNG12 |
| | | | | | SLC25A19 | DCST1 | | ANO6 | SH2B2 |
| | | | | | CYP2S1 | BAIAP2 | | MAMLD1 | FPR1 |
| | | | | | TRPM2 | SOX12 | | C8G | PLXNA4 |
| | | | | | MAN1C1 | | | ANKRD44 | LHFPL2 |
| | | | | | PEX11G | | | PLEKHB1 | ATP6V0A1 |
| | | | | | NDUFC1 | | | TSSK4 | ZDHHC14 |
| | | | | | CELSR2 | | | CALCRL | C2CD2 |
| | | | | | CREB3L4 | | | MGLL | HSD17B6 |
| | | | | | MEGF6 | | | MFSD6L | CD300C |
| | | | | | MPP5 | | | QPCT | MEGF8 |
| | | | | | TMEM97 | | | NDFIP2 | LYZ |
| | | | | | ECE2 | | | MS4A6A | GRASP |
| | | | | | GCNT2 | | | MARCO | MAPK15 |
| | | | | | SVIL | | | CLCN1 | BTK |
| | | | | | SARNP | | | SEMA3B | PLOD3 |
| | | | | | PDIA5 | | | C2CD2 | NTSR1 |
| | | | | | ADAM22 | | | GPRIN1 | LAPTM4B |
| | | | | | ALS2 | | | CLN6 | TIAM1 |
| | | | | | MGP | | | PLXDC2 | PGLYRP2 |
| | | | | | H6PD | | | KCNH3 | CHRNA10 |
| | | | | | PIGK | | | APOO | CCR8 |
| | | | | | DENND1A | | | ZNF599 | KCNH3 |
| | | | | | BCL2 | | | B3GALT2 | AIF1 |
| | | | | | CHMP5 | | | FBXO2 | LGALS3BP |
| | | | | | CRB3 | | | GLT1D1 | PRRG4 |
| | | | | | KCNQ1 | | | LAPTM4B | SV2A |
| | | | | | FAM171A1 | | | CTLA4 | FAR2 |
| | | | | | ZNF532 | | | CCR1 | LTK |
| | | | | | MTUS1 | | | CXADR | RALGPS2 |
| | | | | | ISM1 | | | FITM2 | SVIL |
| | | | | | DYNC2H1 | | | RASGRF2 | CALCRL |
| | | | | | NLRP2 | | | MAP2K6 | PCYOX1L |
| | | | | | AP2A2 | | | DRD3 | TSSK4 |
| | | | | | SERPINH1 | | | S100A8 | MSRB3 |
| | | | | | DCST1 | | | TPCN1 | CYB561D1 |
| | | | | | APOL1 | | | BCL7A | SLC24A4 |
| | | | | | AIG1 | | | IL17C | EPHB3 |
| | | | | | LAMP3 | | | ZDHHC14 | IL10 |
| | | | | | ADCK5 | | | LAT2 | TNFSF13B |
| | | | | | IL6R | | | KIAA0319L | MGLL |
| | | | | | CASK | | | NOTCH4 | WDR44 |
| | | | | | MPHOSPH9 | | | NTSR1 | LAT2 |
| | | | | | ALCAM | | | GFRA2 | GPM6B |
| | | | | | FAM19A2 | | | HMOX1 | HSPA13 |
| | | | | | ABCD2 | | | RALGPS2 | SEMA6B |
| | | | | | PXMP2 | | | RASL11A | CNTNAP1 |
| | | | | | CNKSR2 | | | B3GNT5 | IL17C |
| | | | | | GDF11 | | | PLOD3 | CTLA4 |
| | | | | | BAIAP2 | | | RGMB | PPL |
| | | | | | LEAP2 | | | GCA | HOOK1 |
| | | | | | | | | CHRNA10 | DDN |
| | | | | | | | | LGALS3BP | PHLPP1 |
| | | | | | | | | GPR75 | PGAP3 |
| | | | | | | | | DHRS12 | JUP |
| | | | | | | | | CD300C | SPRN |
| | | | | | | | | HOMER1 | FCN1 |
| | | | | | | | | HSD17B6 | SLC1A2 |
| | | | | | | | | SRC | RASGRF2 |
| | | | | | | | | FCN1 | FITM2 |
| | | | | | | | | COL9A2 | TMEM170B |
| | | | | | | | | LTK | S100A9 |
| | | | | | | | | ACSM3 | TTYH3 |
| | | | | | | | | TNFSF13B | PRSS22 |
| | | | | | | | | SPON1 | CFP |
| | | | | | | | | SCRIB | SLC38A7 |
| | | | | | | | | IL12A | HMOX1 |
| | | | | | | | | SVIL | |
| | | | | | | | | FXYD2 | |
| | | | | | | | | BIK | |
| | | | | | | | | ALOX5 | |
| | | | | | | | | TIAM1 | |
| | | | | | | | | AIF1 | |
| | | | | | | | | DPP4 | |
| | | | | | | | | CDHR1 | |
| | | | | | | | | ENOX2 | |
| | | | | | | | | SLC1A2 | |
| | | | | | | | | ACTN1 | |
| | | | | | | | | F5 | |
| | | | | | | | | CYB561D1 | |
| | | | | | | | | PGAP3 | |
| | | | | | | | | IMPACT | |
| | | | | | | | | PLXNA4 | |
| | | | | | | | | DUSP6 | |
| | | | | | | | | HSPA13 | |
| | | | | | | | | S100A9 | |
| | | | | | | | | CHIC1 | |
| | | | | | | | | TMEM170B | |
| | | | | | | | | SARNP | |
| | | | | | | | | SEMA6A | |
| | | | | | | | | KCNQ5 | |
| | | | | | | | | LYZ | |
| | | | | | | | | SV2A | |
| | | | | | | | | SLC38A7 | |
| | | | | | | | | LGMN | |
| | | | | | | | | ATP8B3 | |
| | | | | | | | | TBXAS1 | |
| | | | | | | | | IFT140 | |
| | | | | | | | | CCDC136 | |
| | | | | | | | | LYSMD4 | |
| | | | | | | | | TTYH3 | |
| | | | | | | | | SPRN | |
| | | | | | | | | CFP | |
| | | | | | | | | JSRP1 | |
| | | | | | | | | PTPRS | |
| | | | | | | | | PPL | |

Genes in bold are both surface and secretory targets, unbolded genes are secretory, italicized genes are surface expressing targets.
DE: Differentially expressed genes, SE: surface expressing/secretory target gene.

What is claimed is:

1. A method for treating a neurodegenerative disorder in a subject having differential expression of at least one gene or gene product as set forth in Table 1 or Table 2 comprising:
identifying a subject having differential expression of the at least one gene or gene product by detecting differential expression of at the least one gene or gene product in a sample obtained from the subject;
administering a treatment or therapy for a neurodegenerative disorder to the subject identified as having differential expression of the at least one gene or gene product.

2. The method of claim 1, wherein differential expression comprises expression of the at least one of the genes or gene products as compared to the expression level of the gene or gene product in healthy subject or a control.

3. The method of claims 1 or 2, wherein the neurodegenerative disorder is Alzheimer's Disease (AD), Parkinson's Disease (PD), Tauopathy, Lewy Body Dementia, or Amyotrophic Lateral Sclerosis (ALS) or motor neuron disease.

4. The method of claims 1 or 2, wherein the neurodegenerative disorder is Parkinson's Disease.

5. The method of any one of the preceding claims wherein the gene or gene product is selected from the group of LSMEM1, AIG1, APOL1, ABCD2, CELSR2, LEAP2, GDF11, LYPD8, CALCRL, NTSR1, AC007040.2, OR1L8, CCR1, CFP, TNFSF13B, ADM5, LYZ, LGALS3BP, LMO7, RNF152, KCNH4, ABCC3, FFAR3, CD300LB, COL16A1, CPB2, IL22, IGFBP6, ACAN, KCNQ4, PAQR4, VAMP4, CNIH2, CX3CR1, CCR5, CCR1, TFEB, SNCA, PARK2, PRKN, UBAPIL, septin 5, GDNF receptor, monoamine oxidase S, aquaporin, LAMP3, polo-like kinase 1, myeloperoxidase, or LRRK2.

6. The method of any one of the preceding claims wherein the gene or gene product is selected from the group of LSMEM1, AIG1, APOL1, ABCD2, CELSR2, LEAP2, GDF11, LYPD8, CALCRL, NTSR1, AC007040.2, OR1L8, CCR1, CFP, TNFSF13B, ADM5, LYZ, LGALS3BP, LMO7, RNF152, KCNH4, ABCC3, FFAR3, CD300LB, COL16A1, CPB2, IL22, IGFBP6, ACAN, KCNQ4, PAQR4, VAMP4, or CNIH2.

7. The method of any one of claims 1-5, wherein the gene or gene product is CX3CR1, CCR5 or CCR1.

8. The method of any one of claims 1-5, wherein the gene or gene product is TFEB, SNCA, PARK2, PRKN, UBAPIL, septin 5, GDNF receptor, monoamine oxidase S, aquaporin, LAMP3, polo-like kinase 1, myeloperoxidase, or LRRK2.

9. The method of claim 8, wherein the gene or gene product is PRKN or LRRK2.

10. The method of claim 8, wherein the gene or gene product is TFEB or UBAPIL.

11. The method of any one of the previous claims, wherein the subject is a mammal.

12. The method of claim 11, wherein the mammal is selected from an equine, bovine, canine, feline, murine, or a human.

13. The method of claim 12, wherein the subject is a human.

14. The method of any one of the preceding claims wherein the treatment or therapy comprises surgery treatment for Parkinson's Disease, or comprises administration of an immunotherapy or an agonist or an antagonist of an immune response.

15. The method of claim 14, wherein the immunotherapy comprises adoptive cell therapy.

16. The method of claim 15, wherein adoptive cell therapy comprises administering a population of engineered cells.

17. The method of claim 14, wherein the antagonist or agonist comprises an antibody, a small molecule, a protein, a peptide, an antisense nucleic acid or an aptamer, including an antibody-small molecule conjugate, a bispecific antibody or bispecific molecule.

18. The method of any one of claims 1-14, wherein the treatment or therapy comprises administration of an anti-TNF therapy.

19. The method of any one of claims 1-14, wherein the treatment comprises administration of a dopamine promoter, an antidepressant, a cognition-enhancing medication, an anti-tremor medication, an anticholinergic, a Mao-B inhibitor, or a COMT inhibitor.

20. The method of claim 1, wherein the sample comprises a blood sample.

21. The method of claim 1, wherein the sample comprises a peripheral blood mononuclear cell (PBMCs), a CD4 memory T cell, or a CD8 memory T cell.

22. The method of claim 1, wherein the step of identifying comprises determining the level of expression of one or more RNA or genes listed in Table 3 or Table 4.

23. The method of claim 22, wherein the expression of the one or more RNA or gene or protein product thereof is at least 2.5 fold, at least 3 fold, at least 3.5 fold, at least 4.5 fold, at least 5 fold, at least 6 fold, at least 7 fold, at least 8 fold, at least 9 fold, at least 10 fold, at least 11 fold, at least 12 fold, at least 13 fold, at least 14 fold, or at least 15 fold, compared to a control sample.

24. The method of claim 22, comprising determining the expression level of one or more of two or more, three or more, or four or more, or five or more, or six or more, or seven or more, or eight or more, or nine or more, or ten or more, or eleven or more, or twelve or more, or thirteen or more, or fourteen or more, or fifteen or more, or sixteen or more, or seventeen or more, or eighteen or more, or nineteen or more, or twenty or more, or twenty-one or more, or twenty-two or more, or twenty-three or more, or all of the RNAs or genes or protein products thereof.

25. The method of claim 1, wherein the differential expression of the gene is determined by a method comprising measuring mRNA encoding the protein, in situ hybridization, northern blot, PCR, quantitative PCR, RNA-seq, a microarray, differential gene expression analysis (DEseq), gene set enrichment analysis (GSEA), comprises surfaceome analysis or secretome analysis.

26. A method for treating a neurodegenerative disorder in a subject having differential expression of at least one of LMO7, LSMEM1, AIG1, APOL1, ABCD2, CELSR2, LEAP2, GDF11, or LYPD8 comprising:
identifying a subject having differential expression of the at least one gene or gene product by detecting differential expression of at least one of LMO7, LSMEM1, AIG1, APOL1, ABCD2, CELSR2, LEAP2, GDFI 1, or LYPD8 in a sample obtained from the subject;
administering a treatment or therapy for a neurodegenerative disorder to the subject identified as having differential expression of the at least one gene or gene product.

27. The method of claim 26, wherein the differential expression comprises the upregulation of LMO7, LSMEM1, AIG1, APOL1, ABCD2, CELSR2, LEAP2, GDF11, or LYPD8 in a sample of CD4 T cells obtained from the subject compared to expression in a control sample.

28. A method for treating a neurodegenerative disorder in a subject having differential expression of at least one of LMO7, CALCRL, NTSR1, AC007040.2, OR1L8, CCR1, CFP, TNFSF13B, ADM5, LYZ, or LGALS3BP comprising:
identifying a subject having differential expression of the at least one gene or gene product by detecting differential expression of at least one of LMO7, CALCRL, NTSR1, AC007040.2, OR1L8, CCR1, CFP, TNFSF13B, ADM5, LYZ, or LGALS3BP in a sample obtained from the subject;
administering a treatment or therapy for a neurodegenerative disorder to the subject identified as having differential expression of the at least one gene or gene product.

29. The method of claim 28, wherein the differential expression comprises the upregulation of LMO7, CALCRL, NTSR1, AC007040.2, OR1L8, CCR1, CFP, TNFSF13B, ADM5, LYZ, or LGALS3BP in a sample of CD8 T cells obtained from the subject.

30. A method for treating a neurodegenerative disorder in a subject having differential expression of at least one of RNF152, KCNH4, ABCC3, FFAR3, CD300LB, COL16A1, CPB2, IL22, IGFBP6, or ACAN comprising:
identifying a subject having differential expression of the at least one gene or gene product by detecting differential expression of at least one of RNF152, KCNH4, ABCC3, FFAR3, CD300LB, COL16A1, CPB2, IL22, IGFBP6, or ACAN in a sample obtained from the subject;
administering a treatment or therapy for a neurodegenerative disorder to the subject identified as having differential expression of the at least one gene or gene product.

31. The method of claim 30, wherein the differential expression comprises the downregulation of RNF152, KCNH4, ABCC3, FFAR3, CD300LB, COL16A1, CPB2, IL22, IGFBP6, or ACAN in a sample of CD4 T cells obtained from the subject.

32. A method for treating a neurodegenerative disorder in a subject having differential expression of at least one of KCNQ4, PAQR4, VAMP4 or CNIH2 comprising:
identifying a subject having differential expression of the at least one gene or gene product by detecting differential expression of at least one of KCNQ4, PAQR4, VAMP4 or CNIH2 in a sample obtained from the subject;
administering a treatment or therapy for a neurodegenerative disorder to the subject identified as having differential expression of the at least one gene or gene product.

33. The method of claim 32, wherein the differential expression comprises the downregulation of KCNQ4, PAQR4, VAMP4 or CNIH2 in a sample of CD8 T cells obtained from the subject.

34. A method for treating a neurodegenerative disorder in a subject identified as having differential expression of at least one of the genes or gene products selected from the group of LSMEM1, AIG1, APOL1, ABCD2, CELSR2, LEAP2, GDF11, LYPD8, CALCRL, NTSR1, AC007040.2, OR1L8, CCR1, CFP, TNFSF13B, ADM5, LYZ, LGALS3BP, LMO7, RNF152, KCNH4, ABCC3, FFAR3, CD300LB, COL16A1, CPB2, IL22, IGFBP6, ACAN, KCNQ4, PAQR4, VAMP4, or CNIH2 comprising administering a treatment or therapy for the neurodegenerative disorder to the subject.

35. A method for treating a neurodegenerative disorder in a subject having differential expression of at least one of the genes or gene products selected from the group of CX3CR1, CCR5 or CCR1, comprising administering a treatment or therapy for a neurodegenerative disorder to the subject.

36. A method for treating a neurodegenerative disorder in a subject having differential expression of at least one of the genes or gene products selected from the group of TFEB, SNCA, PARK2, PRKN, UBAPIL, septin 5, GDNF receptor, monoamine oxidase S, aquaporin, LAMP3, polo-like kinase 1, myeloperoxidase, or LRRK2, comprising administering a treatment or therapy for a neurodegenerative disorder to the subject.

37. A method for treating a neurodegenerative disorder in a subject having differential expression of at least one of the genes or gene products selected from the group of PRKN, LRRK2, TFEB or UBAPIL, comprising administering a treatment or therapy for a neurodegenerative disorder to the subject.

38. A method for treating a neurodegenerative disorder in a subject having differential expression of at least one of the genes or gene products selected from the group of PRKN or LRRK2, comprising administering a treatment or therapy for a neurodegenerative disorder to the subject.

39. A method for treating a neurodegenerative disorder in a subject having differential expression of at least one of the genes or gene products selected from the group of TFEB or UBAPIL, comprising administering a treatment or therapy for a neurodegenerative disorder to the subject.

40. A method for treating a neurodegenerative disorder in a subject having differential expression of CCR5, comprising administering a treatment or therapy for a neurodegenerative disorder to the subject.

41. The method of claim 40, further comprising the step of detecting CCR5 in a sample of PBMCs obtained from the subject.

42. A method for treating a neurodegenerative disorder in a subject having differential expression of CX3CR1, comprising administering a treatment or therapy for a neurodegenerative disorder to the subject.

43. The method of claim 42, further comprising the step of detecting CX3CR1 in a sample of memory CD4 T cells obtained from the subject.

44. A method for treating a neurodegenerative disorder in a subject having differential expression of CCR1, comprising administering a treatment or therapy for a neurodegenerative disorder to the subject.

45. The method of claim 45, further comprising the step of detecting CCR1 in a sample of memory CD8 T cells obtained from the subject.
